(19) United States
(12) Patent Application Publication
(10) Pub. No.: US 2022/0251218 A1
(43) Pub. Date: Aug. 11, 2022

(54) PHARMACEUTICAL COMBINATION AND METHOD FOR OVERCOMING IMMUNE SUPPRESSION OR STIMULATING IMMUNE RESPONSE AGAINST CANCER

(71) Applicant: GNT BIOTECH & MEDICALS CORPORATION, Taipei City (TW)

(72) Inventors: Cheng-Han CHOU, Taipei City (TW); Yi-Hong WU, Taipei City (TW); Jia-Shiong CHEN, Taipei City (TW); Ye-Su CHAO, Taipei City (TW); Chia-Nan CHEN, Taipei City (TW)

(21) Appl. No.: 17/173,129

(22) Filed: Feb. 10, 2021

(51) Int. Cl. C07K 16/28; A61K 31/4406; A61K 31/47; A61K 31/4365; A61K 45/06

(52) U.S. Cl. CPC C07K 16/2863; A61K 31/4406; A61K 45/06; A61K 31/4365; A61K 31/47

(57) ABSTRACT

The invention relates to a method of overcoming immune suppression in tumor microenvironment or stimulating immune response against cancer, comprising administering to a subject a combination of a histone deacetylase (HDAC) inhibitor and a tyrosine kinase inhibitor (TKI).

### FIELD OF THE INVENTION

The present invention relates to immunotherapy. Particularly, the present invention provides a pharmaceutical combination and its applications in regulating tumor microenvironment and cancer immunotherapy.

### BACKGROUND OF THE INVENTION

The new era of cancer treatment in tumor immunology will provide great advancements in the use of immune-oncology (IO) therapy to boost anti-cancer immune response. Immune checkpoint inhibitors (ICIs) are one of the most promising IO therapies that can unleash the power of cytotoxic T lymphocytes (CTLs) to efficiently attack and kill tumors, especially the ICIs targeting PD-1 (Programmed cell death protein 1)/PD-L1 (Programmed death-ligand 1) axis blockade. To date, several ICIs have been developed. However, only about 20% of patients respond to anti-PD-1/anti-PD-L1 antibody monotherapy. About 80% of patients gain no clinical benefit caused by primary and acquired resistance. Resistance to PD-1/PD-L1 blockade is therefore a very important issue to overcome in immunotherapy.

The primary resistance refers to the condition where no responses occur by the PD-1/PD-L1 blockade. In comparing immunotherapy with chemotherapy or targeting therapy, immunotherapy has relatively high rates of primary resistance, and so the clinical benefit is restricted. It is estimated that about 60% of patients receiving immunotherapy have primary resistance. However, acquired resistance refers to the condition where an initial response to PD-1/PD-L1 blockade occurs with the progression of a disease, and a relapse occurs eventually. It is estimated that about 20% of patients receiving immunotherapy have acquired resistance. The low response rates and primary or acquired resistance to PD-1/PD-L1 blockade may be related to the tumor microenvironment (TME) (Annals of Oncology, Volume 27, Issue 8, August 2016, Pages 1492-1504). The TME is a dynamic and complicated composition that controls tumor immune response. The major mechanisms of primary or acquired resistance of PD-1/PD-L1 blockade may include several factors such as TME status, PD-L1 expression, tumor neoantigen expression and presentation, cell signal pathway, immune gene expression, and epigenetic modification.

Numerous combined therapeutic strategies hope to overcome the problem of drug resistance by PD-1/PD-L1 blockade. Many approaches focus on increasing the sensitivity to PD-1/PD-L1 blockade by using anti-PD-1 or anti-PD-L1 antibody in combination with other agents. However, these drug combinations cannot achieve the desired therapeutic benefits, and the efficacy and safety thereof are questionable.

### SUMMARY OF THE INVENTION

The inventors surprisingly found that tyrosine kinase inhibitors (TKIs) plus histone deacetylase (HDAC) inhibitors significantly improve the anti-cancer efficacy via modulation of TME. Furthermore, the TKIs plus HDAC inhibitors combined with ICIs significantly overcome the primary or acquired resistance by PD-1/PD-L1 blockade, and boost the efficacy of immunotherapy.

In one aspect, the present disclosure provides a method for inhibiting or treating a cancer in a subject through overcoming immune suppression in a tumor microenvironment or stimulating immune response against cancer, comprising administering to the subject a combination comprising a histone deacetylase (HDAC) inhibitor or a pharmaceutically acceptable salt thereof and a tyrosine kinase inhibitor (TKI) or a pharmaceutically acceptable salt thereof; wherein the HDAC inhibitor or a pharmaceutically acceptable salt thereof and a tyrosine kinase inhibitor or a pharmaceutically acceptable salt thereof are formulated in a medicament, or the HDAC inhibitor and a tyrosine kinase inhibitor are each formulated as single medicaments for simultaneous, separate or sequential administration.

In another aspect, the present disclosure provides a pharmaceutical combination for use in a method for inhibiting or treating a cancer in a subject through overcoming immune suppression in a tumor microenvironment or stimulating immune response against cancer, wherein the combination comprises a histone deacetylase (HDAC) inhibitor or a pharmaceutically acceptable salt thereof and a tyrosine kinase inhibitor (TKI) or a pharmaceutically acceptable salt thereof; wherein the HDAC inhibitor or a pharmaceutically acceptable salt thereof and a tyrosine kinase inhibitor or a pharmaceutically acceptable salt thereof are formulated in a medicament, or the HDAC inhibitor and a tyrosine kinase inhibitor are each formulated as single medicaments for simultaneous, separate or sequential administration.

In another aspect, the present disclosure also provides a pharmaceutical combination comprises a histone deacetylase (HDAC) inhibitor or a pharmaceutically acceptable salt thereof and a tyrosine kinase inhibitor (TKI) or a pharmaceutically acceptable salt thereof; wherein the HDAC inhibitor or a pharmaceutically acceptable salt thereof and a tyrosine kinase inhibitor or a pharmaceutically acceptable salt thereof are formulated in a medicament, or the HDAC inhibitor and a tyrosine kinase inhibitor are each formulated as single medicaments for simultaneous, separate or sequential administration. In some embodiments of the disclosure, the amounts of the HDAC inhibitor and the TKI in the pharmaceutical combination range from about 10% (w/w) to about 70% (w/w) and about 10% (w/w) to about 70% (w/w), respectively. In a further embodiment, the pharmaceutical combination further comprises an immune checkpoint inhibitor. In some embodiments of the disclosure, the amount of immune checkpoint inhibitor in the combination ranges from about 0.5% (w/w) to about 20% (w/w).

In another aspect, the present disclosure provides a use of a combination comprising of a histone deacetylase (HDAC) inhibitor or a pharmaceutically acceptable salt thereof and a tyrosine kinase inhibitor (TKI) or a pharmaceutically acceptable salt thereof in the manufacture of a single medicament or multiple medicaments for inhibiting or treating a cancer in a subject through overcoming immune suppression in tumor microenvironment or stimulating immune response, wherein the HDAC inhibitor or a pharmaceutically acceptable salt thereof and a tyrosine kinase inhibitor or a pharmaceutically acceptable salt thereof are formulated in a medicament, or the HDAC inhibitor and a tyrosine kinase inhibitor are each formulated as single medicaments for simultaneous, separate or sequential administration.

In some embodiments of the disclosure, the amounts of the HDAC inhibitor and the TKI in the combination described herein range from about 10% (w/w) to about 70% (w/w) and about 10% (w/w) to about 70% (w/w), respectively.

In one embodiment, the present disclosure provides a method for treating a cancer in a subject through overcoming immune suppression in a tumor microenvironment or stimulating immune response, comprising administering to the subject a combination comprising a histone deacetylase (HDAC) inhibitor or a pharmaceutically acceptable salt thereof, a tyrosine kinase inhibitor (TKI) or a pharmaceutically acceptable salt thereof, in combination with an immune checkpoint inhibitor (ICI); wherein the histone deacetylase (HDAC) inhibitor or a pharmaceutically acceptable salt thereof, the tyrosine kinase inhibitor (TKI) or a pharmaceutically acceptable salt thereof and the immune checkpoint inhibitor are formulated in a medicament or one or two of the HDAC inhibitor or a pharmaceutically acceptable salt thereof, tyrosine kinase inhibitor or a pharmaceutically acceptable salt thereof and immune checkpoint inhibitor are formulated as multiple medicaments for simultaneous, separate or sequential administration.

In another embodiment, the present disclosure provides a pharmaceutical combination for use in a method for treating a cancer in a subject through overcoming immune suppression in a tumor microenvironment or stimulating immune response against cancer, wherein the combination comprises a histone deacetylase (HDAC) inhibitor or a pharmaceutically acceptable salt thereof, a tyrosine kinase inhibitor (TKI) or a pharmaceutically acceptable salt thereof, in combination with an immune checkpoint inhibitor; wherein the histone deacetylase (HDAC) inhibitor or a pharmaceutically acceptable salt thereof, the tyrosine kinase inhibitor (TKI) or a pharmaceutically acceptable salt thereof and the immune checkpoint inhibitor (ICI) are formulated in a medicament or one or two of the HDAC inhibitor or a pharmaceutically acceptable salt thereof, tyrosine kinase inhibitor or a pharmaceutically acceptable salt thereof and immune checkpoint inhibitor are formulated as multiple medicaments for simultaneous, separate or sequential administration.

In another embodiment, the present disclosure provides a use of a combination in the manufacture of a single medicament or multiple medicaments for inhibiting or treating a cancer in a subject through overcoming immune suppression in tumor microenvironment or stimulating immune response, wherein the combination comprises a histone deacetylase (HDAC) inhibitor or a pharmaceutically acceptable salt thereof, a tyrosine kinase inhibitor (TKI) or a pharmaceutically acceptable salt thereof, in combination with an immune checkpoint inhibitor (ICI); wherein the histone deacetylase (HDAC) inhibitor or a pharmaceutically acceptable salt thereof, the tyrosine kinase inhibitor (TKI) or a pharmaceutically acceptable salt thereof and the immune checkpoint inhibitor are formulated in a medicament or one or two of the HDAC inhibitor or a pharmaceutically acceptable salt thereof, tyrosine kinase inhibitor or a pharmaceutically acceptable salt thereof and immune checkpoint inhibitor are formulated as multiple medicaments for simultaneous, separate or sequential administration.

In one embodiment of the disclosure, the amount of immune checkpoint inhibitor in the combination described herein ranges from about 0.5% (w/w) to about 20% (w/w).

In one embodiment, in the combination described herein, the amounts of the histone deacetylase (HDAC) inhibitor or a pharmaceutically acceptable salt thereof, a tyrosine kinase inhibitor (TKI) or a pharmaceutically acceptable salt thereof and the immune checkpoint inhibitor range from about 10% (w/w) to about 70% (w/w), about 10% (w/w) to about 70% (w/w), and about 0.5% (w/w) to about 20% (w/w), respectively.

In some embodiments of the disclosure, the immune checkpoint inhibitor described herein is an anti-cytotoxic T-lymphocyte antigen-4 (CTLA-4) antibody or agent, anti-programmed cell death protein 1 (PD-1) antibody or agent, an anti-programmed death-ligand 1 (PD-L1) antibody or agent, an anti-T-cell immunoglobulin and mucin domain-3 (TIM-3) antibody or agent, anti-B- and T-lymphocyte attenuator (BTLA) antibody or agent, anti-V-domain Ig containing suppressor of T-cell activation (VISTA) antibody or agent, an anti-lymphocyte activation gene-3 (LAG-3) antibody or agent, KIR (killer-cell immunoglobulin-like receptor) inhibitor or antibody, A2AR (adenosine A2A receptor inhibitor, CD276 inhibitor or antibody, or VTCN1 inhibitor or antibody. More preferably, the immune checkpoint inhibitor is pembrolizumab, lambrolizumab, pidilizumab, nivolumab, durvalumab, avelumab, or atezolizumab.

In some embodiments of the disclosure, the cancer described herein includes, but is not limited to, melanoma, head and neck cancer, merkel cell carcinoma, hepatocellular carcinoma, renal cell carcinoma, colorectal cancer, endometrial carcinoma, cervical cancer, esophageal squamous cell carcinoma, small cell lung cancer, non-small cell lung cancer, breast cancer, gastric carcinoma, esophagogastric junction carcinoma, classical Hodgkin lymphoma, Non-Hodgkin lymphoma, urothelial carcinoma, primary mediastinal large B-cell lymphoma, glioblastoma, pancreatic cancer, benign prostate hyperplasia, prostate cancer, ovarian cancer, chronic lymphocytic leukemia, Merkel cell carcinoma, acute myeloid leukemia, gallbladder cancer, cholangiocarcinoma, urinary bladder cancer, and uterine cancer.

In a further embodiment, the caner is an immune checkpoint inhibitor-resistant cancer or a cancer failure to respond to a cancer immunotherapy.

In one embodiment, the subject has not received a cancer therapy. In another embodiment, the subject has received a cancer therapy but failed to the therapy. In some embodiments, the cancer therapy is a radiotherapy, chemotherapy or an immunotherapy. In a further embodiment, the immunotherapy is an anti-PD1 immunotherapy, anti-PD L1 immunotherapy or anti-CTL4 immunotherapy.

In some embodiments of the disclosure, the HDAC inhibitor or a pharmaceutically acceptable salt thereof, as described herein, is a class I-selective HDAC inhibitor or pan-HDAC inhibitor which must inhibit class I HDAC. The examples of the HDAC inhibitor or a pharmaceutically acceptable salt thereof include, but are not limited to, a benzamide class of HDAC inhibitor. Preferably, the HDAC inhibitor is Chidamide, Entinostat, Vorinostat, Romidepsin, Panobinostat, Belinostat, Valproic acid, Mocetinostat, Abexinostat, Pracinostat, Resminostat, Givinostat, Quisinostat, Domatinostat, Quisnostat, CUDC-101, CUDC-907, Pracinostat, Citarinostat, Droxinostat, Abexinostat, Ricolinostat, Tacedinaline, Fimepinostat, Tubacin, Resminostat, ACY-738, Tinostamustine, Tubastatin A, Givinostat and Dacinostat.

In some embodiments of the disclosure, the TKI or a pharmaceutically acceptable salt thereof, as described herein, is an inhibitor of receptor tyrosine kinases. Preferably, the TKI or a pharmaceutically acceptable salt thereof, as described herein, is an inhibitor of vascular endothelial growth factor receptor (VEGFR). Examples of the TKI or a pharmaceutically acceptable salt thereof include, but are not limited to, Cabozantinib, Regorafenib, Axitinib, Afatinib, Ninetedanib, Crizotinib, Alectinib, Trametinib, Dabrafenib, Sunitinib, Ruxolitinib, Vemurafenib, Sorafenib, Ponatinib, Encorafenib, Brigatinib, Pazopanib, Dasatinib, Imatinib, Lenvatinib, Vandetanib, surufatinib and Sitravatinib.

In some further embodiments, examples of the combination as described herein include, but are not limited to, the following:
(i) anti-CTLA-4, anti-PD1 or anti-PD L1 antibody (ICI), Regorafenib, Cabozantinib, Ibrutinib, Axitinib or a pharmaceutically acceptable salt thereof (TKI) and Chidamide or Chidamide-k30 or a pharmaceutically acceptable salt thereof (HDAC);
(ii) anti-CTLA-4 anti-PD1 or anti-PD L1 antibody (ICI), Regorafenib, Cabozantinib, Ibrutinib, Axitinib or a pharmaceutically acceptable salt thereof (TKI) and Chidamide-HCl salt (HDAC).

In some further embodiments, examples of the combination as described herein include, but are not limited to, the following:
(1) anti-CTLA-4 antibody (ICI)+Regorafenib (TKI)+Chidamide or Chidamide-k30 (HDAC);
(2) anti-CTLA-4 antibody (ICI)+Cabozantinib (TKI)+Chidamide or Chidamide-k30 (HDAC); and
(3) Anti-CTLA-4 antibody (ICI)+Cabozantinib (TKI)+Chidamide-HCl salt (HDAC).

In some embodiments of the disclosure, the method or the combination, as described herein, further comprises administering one or more additional anti-cancer agents.

### BRIEF DESCRIPTION OF THE DRAWINGS

FIGS. 1 (A) to (C) show the consecutive treatment schedule and responsive result of the first-line anti-PD-1 Ab treatment. Male Balb/c mice bearing subcutaneous CT26 tumors (1×10⁶ cell/mice) were treated with a first-line therapy of anti-PD-1 Ab (mean tumor volume: 113 mm³ when treatment began). The mice were administered intraperitoneally (i.p.) with anti-PD-1 Ab or IgG at 2.5 mg/kg, once every 3 days for 3 doses. When the mice responded to anti-PD-1 Ab with tumor shrinking, they were given three more doses. (A) consecutive treatment schedule, (B) Tumor size (mm³) from mice responsive to first-line anti-PD-1 Ab treatment, in comparison with control group treated with anti-IgG antibody for 6 times, (C) Tumor fold change of (B).

FIG. 2(A) to (C) show the results of a second-line treatment in the mice having anti-PD-1 antibody primary resistance. In the first-line anti-PD-1 antibody therapy, if the tumor showed 2.5- to 3-times consecutive increases in tumor volume and with volumes of <600 mm³, the mice were defined as having primary resistance. These mice were subsequently reenrolled and divided into five groups in a second-line treatment for efficacy study. In the second-line treatment, anti-IgG antibody was as a control and the anti-IgG and anti-CTLA-4 antibodies were administered intraperitoneally (i.p.) at 2.5 mg/kg, once every 3 days for 6 doses. The combinations in the second-line treatment are: Anti-CTLA-4 antibody (2.5 mg/kg) combined with Chidamide-HCl salt (50 mg/kg) plus Celecoxib (50 mg/kg); anti-CTLA-4 antibody (2.5 mg/kg) combined with Regorafenib (30 mg/kg) plus Chidamide-k30 (50 mg/kg); and anti-CTLA-4 antibody (2.5 mg/kg) combined with Cabozantinib (30 mg/kg) plus Chidamide-k30 (50 mg/kg). The combinations were administered p.o, once every day for 16 times. (A) Tumor size (mm³), (B) Tumor fold change, (C) Mice body weights.

FIG. 3(A) to (C) show the results of second-line treatment in mice with hyperprogressive disease (HPD) tumor during anti-PD-1 antibody therapy. After three times of administration of first-line Anti-PD-1 antibody, if the tumor volumes were >600 mm³, the mice were defined as having hyperprogressive disease (HPD). These mice were subsequently reenrolled in a second-line treatment for efficacy study. The combination in the second-line treatment is Anti-CTLA-4 antibody (2.5 mg/kg) combined with Cabozantinib (30 mg/kg) plus Chidamide-HCl salt (50 mg/kg). The combination was administered i.p. and p.o. The antibodies were administered i.p. once every 3 days for 6 doses, and Cabozantinib combined with Chidamide-HCl salt were administered once every day for 16 days. (A) Tumor folds change, (B) Mice body weights, (C) The anti-CTLA-4 antibody combined with Cabozantinib plus Chidamide-HCl salt group achieved 2 CR, 9 SD, with the ORR 18.1%.

FIG. 4(A) to (F) show the results of individual tumor volume in the second-line treatment of the anti-PD-1 Ab primary resistance mice (as shown in FIGS. 2 and 3). (A) The anti-IgG antibody group as a control achieved 5 PD, with the ORR 0%. (B) The anti-CTLA-4 antibody group achieved 5 SD and 2 PD, with the ORR 0%. (C) The anti-CTLA-4 antibody combined with Chidamide-HCl salt plus Celecoxib group achieved 3 CR, 4 SD and 1 PD, with the ORR 37.5%. (D) The anti-CTLA-4 antibody combined with Regorafenib plus Chidamide-k30 group achieved 5 CR and 1 PR, 2 SD, with the ORR 62.5%. (E) The anti-CTLA-4 antibody combined with Cabozantinib plus Chidamide-k30 group achieved 3 CR and 1 PR, 3 SD, with the ORR 57.1%. (F) In the HPD mice, the treatment with anti-CTLA-4 antibody combined with Cabozantinib plus Chidamide-HCl salt achieved 2 CR, 9 SD, with the ORR 18.1%.

FIG. 5(A) to (E) show the results of second-line treatment for acquired resistance to the first-line anti-PD-1 antibody treatment. The mice were treated with the first-line treatment of anti-PD-1 antibody intraperitoneally (i.p.) at 2.5 mg/kg, once every 3 days for 6 doses. If the tumor volumes were under 2 folds through 3-times treatments and then over 2-fold increases through all 6-times treatments, the mice were defined as having acquired resistance (relapse) to the anti-PD-1 antibody. These mice were subsequently treated with a second-line treatment of anti-CTLA-4 antibody combined with Regorafenib plus Chidamide-k30 for efficacy study. (A) For comparison, the tumor sizes in different treatments from FIG. 2(A) are shown. (B) The treatment schedule and the tumor sizes of first- and second-line treatment for mice with acquired anti-PD-1 antibody resistance. (C) Individual tumor volume in the mice with acquired anti-PD-1 antibody resistance after second-line treatment with anti-CTLA-4 antibody combined with Regorafenib plus Chidamide-k30 regimen. The treatment achieved 1 CR and 6 SD, with the ORR 14.1%. (D) Overall survival rates after second-line treatment for mice with primary resistance to anti-PD-1 antibody. (E) Overall survival rates after second-line treatment for mice with acquired resistance to anti-PD-1 antibody.

FIG. 6(A) to (G) show the results of antitumor effects and immunity evaluation of Lenvatinib treatment alone or in combination with anti-PD-1 antibody in the CT26 tumor-bearing mice model. Balb/c mice bearing a CT26 tumor were treated with various therapeutic modalities as indicated. Anti-IgG antibody, IgG control (2.5 mg/kg); anti-PD-1 monoclonal antibody, PD-1 (2.5 mg/kg); Lenvatinib (10 mg/kg); anti-PD-1 Ab (2.5 mg/kg) combined with Lenvatinib (10 mg/kg). (A) Total tumor volumes. (B) Individual tumor volumes. (C) Mice body weights. (D) Animal survival rates. (E) Recurrence rates. (F) Experimental design for rechallenge schedule. (G) Mice bearing regressed CT26 tumors (that is, achieving CR or PR response) were rechallenged with CT26 cells on day 34 and evaluated for recurrence on day 51. The rechallenge-induced recurrence is defined as when tumors increase up to as least 2 fold and tumor volume over 300 mm³ as compared to the tumor size on day 41. Recurrence rate after CT26 cells rechallenge is shown in each group. CT26 tumor-bearing mice were treated as indicated and euthanized at a tumor volume of 3000 mm³ after tumor implantation. Data are given as mean±SEM; *P<0.05, **P<0.01, ***P<0.001, ****P<0.0001, one-way ANOVA with Tukey's test.*, compared to IgG; #, compared to PD-1.

FIG. 7(A) and (B) show the results of efficacy comparison of Cabozantinib, Ibrutinib, Axitinib, Olaparib and Chidamide-k30 combined with anti-PD-1 antibody. Balb/c mice bearing a CT26 tumor were treated with various therapeutic modalities as indicated. Anti-IgG antibody, IgG control (2.5 mg/kg); anti-PD-1 monoclonal antibody, PD-1 (2.5 mg/kg); Chidamide-k30 (50 mg/kg); Celecoxib (50 mg/kg); Cabozantinib (30 mg/kg); Ibrutinib (6 mg/kg); Axitinib (12.5 mg/kg); Olaparib (50 mg/kg). (A) Total tumor volumes and mice body weight. (B) Individual tumor volumes. CT26 tumor-bearing mice were treated as indicated and euthanized at a tumor volume of 3000 mm³ after tumor implantation. Data are given as mean±SEM; *P<0.05, **P<0.01, ***P<0.001, ****P<0.0001, one-way ANOVA with Tukey's test.*, compared to IgG; #, compared to PD-1.

FIG. 8(A) to (F) show the results of therapeutic response and immunity evaluation of Cabozantinib plus Celecoxib or Chidamide-k30 combined with anti-PD-1 antibody in CT26 tumor-bearing mice. Balb/c mice bearing a CT26 tumor were treated with various therapeutic modalities as indicated. Anti-IgG antibody, IgG control (2.5 mg/kg); anti-PD-1 monoclonal antibody, PD-1 (2.5 mg/kg); Cabozantinib (30 mg/kg); Chidamide-k30 (50 mg/kg); Celecoxib (50 mg/kg). (A) Total tumor volumes. (B) Individual tumor volumes. (C) Mice body weights. (D) Animal survival rates. (E) Recurrence rates. (F) Mice bearing regressed CT26 tumors were rechallenged with CT26 cells and assessed as described in FIG. 6(G). Recurrence rate after CT26 cells rechallenge is shown in each group. CT26 tumor-bearing mice were treated as indicated and euthanized at a tumor volume of 3000 mm³ after tumor implantation. Data are given as mean±SEM; *P<0.05, **P<0.01, ***P<0.001, ****P<0.0001, one-way ANOVA with Tukey's test.*, compared to IgG; #, compared to PD-1.

FIG. 9(A) to (K) show the results of therapeutic response and immunity evaluation of different tyrosine kinase inhibitors combined with Chidamide-k30 in CT26 tumor-bearing mice. Balb/c mice bearing a CT26 tumor were treated with various therapeutic modalities as indicated. Vehicle, 5% DMSO; Lenvatinib (10 mg/kg); Axitinib (30 mg/kg); Regorafenib (30 mg/kg); Cabozantinib (30 mg/kg); and Chidamide-k30 (50 mg/kg). (A) Total tumor volumes of Levatinib, Axitinib, alone or combined with Chidamide-k30 treatment; (B) individual tumor volumes; (C) mice body weights; (D) animal survival rates; and (E) recurrence rates. (F) Total tumor volumes of Regorafenib, Cabozantinib, alone or combined with Chidamide-k30 treatment; (G) individual tumor volumes, (H) mice body weights, (I) animal survival rates; and (J) recurrence rates. (K) Mice bearing regressed CT26 tumors were rechallenged with CT26 cells and assessed as described in FIG. 6(G). Recurrence rate after CT26 cells rechallenge is shown in each group. CT26 tumor-bearing mice were treated as indicated and euthanized at a tumor volume of 3000 mm³ after tumor implantation. Data are given as mean±SEM; *P<0.05, **P<0.01, ***P<0.001, ****P<0.0001, one-way ANOVA with Tukey's test.*, compared to IgG; #, compared to PD-1. The p value of overall survival was determined using Log-rank (Mantel-Cox) test, comparing each group, *P<0.05, **P<0.01, ***P<0.001, ****P<0.0001.

FIG. 10(A) to (K) show the results of therapeutic response and immunity evaluation of Cabozantinib and Regorafenib combined with Chidamide-k30 plus anti-PD-1 antibody in CT26 tumor-bearing mice. Balb/c mice bearing a CT26 tumor were treated with various therapeutic modalities as indicated. Anti-IgG antibody, IgG control (2.5 mg/kg); anti-PD-1 monoclonal antibody, PD-1 (2.5 mg/kg); Cabozantinib (30 mg/kg); Regorafenib (30 mg/kg); Chidamide-k30 (50 mg/kg). The combination with Cabozantinib is shown in (A) total tumor volumes, (B) individual tumor volumes, (C) mice body weights, (D) animal survival rates, and (E) recurrence rates. However, the combination with Regorafenib is shown in (F) total tumor volumes, (G) individual tumor volumes, (H) mice body weights, (I) animal survival rates and (J) recurrence rates. (K) Mice bearing regressed CT26 tumors were rechallenged with CT26 cells and assessed as described in FIG. 6(G). Recurrence rate after CT26 cells rechallenge is shown in each group. CT26 tumor-bearing mice were treated as indicated and euthanized at a tumor volume of 3000 mm³ after tumor implantation. Data are given as mean±SEM; *P<0.05, **P<0.01, ***P<0.001, ****P<0.0001, one-way ANOVA with Tukey's test.*, compared to IgG; #, compared to PD-1. The p value of overall survival was determined using Log-rank (Mantel-Cox) test, comparing each two group, *P<0.05, **P<0.01, ***P<0.001, ****P<0.0001.

FIG. 11(A) to (T) show the results of therapeutic response of ICIs combined with TKIs plus HDAC inhibitors (HDACis) in CT26-bearing mice. Balb/c mice bearing a CT26 tumor were treated with various therapeutic modalities as indicated. IgG, anti-IgG Ab as control (2.5 mg/kg); anti-PD-1 monoclonal antibody, PD-1 (2.5 mg/kg); anti-PD-L1 monoclonal antibody (2.5 mg/kg), PD-L1 (2.5 mg/kg); anti-CTLA-4 monoclonal antibody (2.5 mg/kg), CTLA-4 (2.5 mg/kg); Regorafenib (30 mg/kg); Cabozantinib (30 mg/kg); Chidamide-k30 (50 mg/kg); Vorinostat (150 mg/kg); Entinostat (20 mg/kg); RMC-4550 (30 mg/kg). First, evaluating anti-PD-1 Ab combined with Regorafenib plus different HDAC inhibitors in CT26 tumor-bearing mice. (A) Total tumor volumes, (B) individual tumor volumes, (C) mice body weights, (D) animal survival rates and recurrence rates were recorded. Second, evaluating anti-PD-1 Ab combined with Cabozantinib plus different HDAC inhibitors in CT26-bearing mice. (E) Total tumor volumes, (F) individual tumor volumes, (G) mice body weights, (H) animal survival rates and recurrence rates were recorded. Third, evaluating the different ICIs combined with Regorafenib plus Chidamide-k30 in CT26-bearing mice. (I) Total tumor volumes, (J) individual tumor volumes, (K) mice body weights, (L) animal survival rates and recurrence rates were recorded. Fourth, evaluating the different ICIs combined with Cabozantinib plus Chidamide-k30 in CT26-bearing mice. (M) Total tumor volumes, (N) individual tumor volumes, (0) mice body weights, (P) animal survival rates and recurrence rates were recorded. Fifth, evaluating the anti-PD-1 Ab combined with different TKIs plus Chidamide-k30 in CT26-bearing mice. (Q) Total tumor volumes, (R) individual tumor volumes, (S) mice body weights, (T) animal survival rates and recurrence rates were recorded. CT26 tumor-bearing mice were treated as indicated and euthanized at a tumor volume of 3000 mm³ after tumor implantation. Data are given as mean±SEM; *P<0.05, **P<0.01, ***P<0.001, ****P<0.0001, one-way ANOVA with Tukey's test.*, compared to IgG; #, compared to PD-1. The p value of overall survival was determined using Log-rank (Mantel-Cox) test, comparing each group, *P<0.05, **P<0.01, ***P<0.001, ****P<0.0001.

FIG. 12 (A) and (B) show the results of an immune cell population analysis of lymphocytes and myeloid-derived MDSCs in the CT26-bearing mice tumor. The CT26 tumor-bearing mice were treated with anti-IgG Ab as control, IgG (2.5 mg/kg); anti-PD-1 monoclonal antibody, PD-1 (2.5 mg/kg); Cabozantinib (30 mg/kg); Regorafenib (30 mg/kg); Chidamide-k30 (50 mg/kg). The CT26 tumor-bearing mice were treated with various therapeutic modalities as indicated. Tumor samples were isolated on day 9th after treatment for analyzing cell population in tumors. (A) Tumor size of each treatment group and the results of flow cytometry of CD3, CD4, CD8, and Treg cell population in tumors. Results are shown as mean±SD. *p<0.05 vs. anti-IgG Ab, (n=8-12). (B) Results of flow cytometry of myeloid-derived CD11b, PMN-MDSC, M-MDSC, and tumor macrophage cell populations in tumors. Results are shown as mean±SD. *p<0.05 vs. anti-IgG Ab. (n=8-12).

FIG. 13(A) to (D) show that the resistance to the first-line anti-PD-1 Ab treatment was overcome by Chidamide-HCl salt/Chidamide-k30 combined with Cabozantinib/Regorafenib plus anti-CTLA-4 Ab via regulation of gene expression in the TME in CT-26 tumor-bearing mice. Tumors were analyzed on day 13 after starting the second line treatment for gene expression by RNA-seq. Heatmap of gene expression related to (A) interferon gamma, (B) interferon beta, (C) T cell mediated cytotoxicity, and (D) angiogenesis activity with scores. NES: normalized enrichment score; FDR: false discovery rates. Signature scores were calculated by mean log 2 (TPM) of their respective member genes; P-values: Mann-Whitney test, two-tailed. TPM, transcripts per million; DGE, differential gene expression.

FIG. 14(A) to (E) show that Chidamide is a key component in the regimens of anti-PD-1 Ab combined with Regorafenib/Cabozantinib plus Chidamide-k30 that significantly regulates gene expression in TME of CT26 tumors-bearing mice. Tumors were analyzed on day 9 after starting treatment for gene expression by RNA-seq. Heatmap of gene expression related to (A) chemokine activity, (B) immune response, (C) interferon gamma, (D) transmembrane receptor protein tyrosine kinase activity, and (E) angiogenesis activity are shown with scores. NES: normalized enrichment score; FDR: false discovery rates. Signature scores were calculated by mean log 2 (TPM) of their respective member genes; P-values: Mann-Whitney test, two-tailed. TPM, transcripts per million; DGE, differential gene expression.

FIGS. 15 (A) to (L) show the results of therapeutic response of TKIs plus HDAC inhibitors (Chidamide-k30) combined with or without anti-PD-1 antibody in CT26-bearing mice. Balb/c mice bearing a CT26 tumor were treated with various therapeutic modalities as indicated. IgG, anti-IgG Ab as control (2.5 mg/kg); Vehicle, 5% DMSO; PD-1, anti-PD-1 monoclonal antibody (2.5 mg/kg); Regorafenib (30 mg/kg); Cabozantinib (30 mg/kg); Chidamide-k30 (50 mg/kg); Sitravatinib (20 mg/kg); Celecoxib (50 mg/kg). First, evaluating anti-PD-1 Ab combined with Regorafenib with or without Chidamide-k30 in CT26 tumor-bearing mice. (A) Total tumor volumes, (B) individual tumor volumes, (C) mice body weights, (D) animal survival rates and recurrence rates were recorded. Second, evaluating anti-PD-1 Ab combined with Cabozantinib with or without Chidamide-k30 in CT26-bearing mice. (E) Total tumor volumes, (F) individual tumor volumes, (G) mice body weights, (H) animal survival rates and recurrence rates were recorded. Third, evaluating different TKIs combined with Chidamide-k30 in CT26-bearing mice. (I) Total tumor volumes, (J) individual tumor volumes, (K) mice body weights, (L) animal survival rates and recurrence rates were recorded. CT26 tumor-bearing mice were treated as indicated and euthanized at a tumor volume of 3000 mm³ after tumor implantation. Data are given as mean±SEM; *P<0.05, **P<0.01, ***P<0.001, ****P<0.0001, one-way ANOVA with Tukey's test.*, compared to IgG; #, compared to PD-1. The p value of overall survival was determined using Log-rank (Mantel-Cox) test, comparing each group, *P<0.05, **P<0.01, ***P<0.001, ****P<0.0001.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned are incorporated herein by reference.

The term “a” and “an” refers to one or to more than one (i.e., to at least one) of the grammatical object of the article. By way of example, “an element” means one element or more than one element. The use of “or” means “and/or,” unless specifically stated otherwise.

As used herein, “subject,” “individual” and “patient” are used interchangeably to refer to a vertebrate, preferably a mammal, more preferably a human. Mammals include, but are not limited to, murines, simians, humans, farm animals, sport animals, and pets. Tissues, cells and their progeny of a biological entity obtained in vitro or cultured in vitro are also encompassed.

As used herein, “therapeutically effective amount” means an amount sufficient to treat a subject afflicted with a disease (e.g., a neurodegenerative disease) or to alleviate a symptom or a complication associated with the disease.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the term “immunotherapy” refers to the treatment of a subject afflicted with, or at risk of contracting or suffering a recurrence of a disease by a method comprising inducing, enhancing, suppressing or otherwise modifying an immune response.

As used herein, the term “programmed cell death protein 1 (PD-1)” refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

As used herein, the term “programmed death-ligand1 (PD-L1)” is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

As used herein, an “antibody” and “antigen-binding fragments thereof” encompass naturally occurring immunoglobulins (e.g., IgM, IgG, IgD, IgA, IgE, etc.) as well as non-naturally occurring immunoglobulins, including, for example, single chain antibodies, chimeric antibodies (e.g., humanized murine antibodies), heteroconjugate antibodies (e.g., bispecific antibodies), Fab′, F(ab′).sub.2, Fab, Fv, and rIgG. As used herein, an “antigen-binding fragment” is a portion of the full-length antibody that retains the ability to specifically recognize the antigen, as well as various combinations of such portions.

As used herein, the term “cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” as used herein refers to primary, metastatic and recurrent cancers.

As used herein, the term “combination”, “therapeutic combination” or “pharmaceutical combination”, as used herein, defines either a fixed combination in one dosage unit form or a kit of parts for the combined administration where Compound A and Compound B may be administered independently at the same time or separately within time intervals.

As used herein, the term “pharmaceutically acceptable” is defined herein to refer to those compounds, materials, compositions and/or dosage forms, which are, within the scope of sound medical judgment, suitable for contact with the tissues a subject, e.g., a mammal or human, without excessive toxicity, irritation allergic response and other problem complications commensurate with a reasonable benefit/risk ratio.

As used herein, the term “co-administration” or “combined administration” as used herein is defined to encompass the administration of the selected therapeutic agents to a single patient, and are intended to include treatment regimens in which the agents are not necessarily administered by the same route of administration or at the same time.

The present disclosure develops methods and combinations that focus on the regulation of tumor microenvironment components, thereby removing immune suppression in a tumor microenvironment or stimulating an immune system against cancer cells. The tumor microenvironment is an important aspect of cancer biology that contributes to tumor initiation, tumor progression and responses to therapy. The tumor microenvironment is composed of a heterogeneous cell population that includes malignant cells and cells that support tumor proliferation, invasion, and metastatic potential through extensive crosstalk. Tumor cells often induce an immunosuppressive microenvironment, which favors the development of immunosuppressive populations of immune cells, such as myeloid-derived suppressor cells (MDSCs) and regulatory T cells (Tregs). Therefore, targets within the tumor microenvironment have been uncovered that can help direct and improve the actions of various cancer therapies, notably immunotherapies that work by potentiating host anti-cancer immune responses. The method and combinations not only provide advantageous effect but also synergistic effect in inhibiting or treating a cancer.

Accordingly, the first aspect of the present disclosure is to provide a method of overcoming immune suppression in a tumor microenvironment or stimulating immune response against cancer naïve or resistant to first-line immune checkpoint inhibitor therapy, comprising administering to a subject a combination of a histone deacetylase inhibitor and a tyrosine kinase inhibitor. In one embodiment, the method comprises administering to the subject a combination of the HDAC inhibitor and the TKI in combination with an immune checkpoint inhibitor. Alternatively, the present disclosure provides a use of a pharmaceutical combination of an HDAC inhibitor and a TKI in the manufacture of a medicament for overcoming immune suppression in a tumor microenvironment or stimulating an immune response against cancer. Alternatively, the present disclosure provides a pharmaceutical combination for overcoming immune suppression in a tumor microenvironment or stimulating immune response against cancer, wherein the pharmaceutical combination comprises an HDAC inhibitor and a TKI. Preferably, the pharmaceutical combination further comprises an immune checkpoint inhibitor.

The second aspect of the present disclosure is to provide a pharmaceutical combination comprising an HDAC inhibitor and a TKI. Preferably, the pharmaceutical combination further comprises an immune checkpoint inhibitor.

In one embodiment, the amounts of the HDAC inhibitor and the TKI in the pharmaceutical combination are about 10% (w/w) to about 70% (w/w) and about 10% (w/w) to about 70% (w/w), respectively.

In some embodiments, the amount of the HDAC inhibitor in the pharmaceutical combination ranges from about 20% (w/w) to about 70% (w/w), about 30% (w/w) to about 70% (w/w), about 40% (w/w) to about 70% (w/w), about 20% (w/w) to about 60% (w/w), about 30% (w/w) to about 60% (w/w), about 40% (w/w) to about 60% (w/w) or about 35% (w/w) to about 60% (w/w).

In some embodiments, the amount of the TKI in the pharmaceutical combination ranges from about 20% (w/w) to about 70% (w/w), about 30% (w/w) to about 70% (w/w), about 40% (w/w) to about 70% (w/w), about 20% (w/w) to about 60% (w/w), about 30% (w/w) to about 60% (w/w), about 40% (w/w) to about 60% (w/w) or about 35% (w/w) to about 60% (w/w).

An HDAC inhibitor possesses very potent epigenetic modulation properties that significantly improve immune modulation activities. HDACs are classes of enzymes catalyzing removal of an acetyl group from lysine on a histone. Such deacetylation leads the histones to wrap DNA more tightly. HDAC inhibition controls chromatin remodeling resulting in regulation of gene expression. HDACs have been shown to be involved in oncogenic transformation by mediated gene expression that influences the cell cycle progression, proliferation, and apoptosis. HDACs are investigated as possible treatment targets for cancers as well as parasitic, infection (such as AIDS), and inflammatory diseases. Based on their homology of accessory domains to yeast histone deacetylases, the 18 currently known human histone deacetylases are classified into four groups (I-IV). Class I, which includes HDAC1, -2, -3 and -8 is related to yeast RPD3 gene; Class IIA includes HDAC4, -5, -7 and -9; Class IIB including HDAC-6 and -10 is related to yeast Hda1 gene; Class III, also known as the sirtuins, is related to the Sir2 gene and includes SIRT1-7; and Class IV, which contains only HDAC11, has features of both Class I and II.

In one embodiment of the present disclosure, the HDAC inhibitor is an inhibitor of class I HDAC or class II HDAC. Preferably, the HDAC inhibitor is a selective inhibitor of class I HDACs. In some embodiments, the HDAC inhibitor is a benzamide class of histone deacetylase (HDAC) inhibitors. In some embodiments, the HDAC inhibitor includes, but is not limited to, Chidamide, Entinostat, Vorinostat, Romidepsin, Panobinostat, Belinostat, Valproic acid, Mocetinostat, Abexinostat, Pracinostat, Resminostat, Givinostat Quisinostat, Domatinostat, Quisnostat, CUDC-101, CUDC-907, Pracinostat, Citarinostat, Droxinostat, Abexinostat, Ricolinostat, Tacedinaline, Fimepinostat, Tubacin, Resminostat, ACY-738, Tinostamustine, Tubastatin A, Givinostat or Dacinostat. In some embodiments, the HDAC inhibitor is Chidamide, Entinostat, Vorinostat, or Mocetinostat.

Tyrosine kinase (TK) is an enzyme catalyzing transferring a phosphate group from ATP to a tyrosine residue. It functions as a switch in cellular functions such as signal transduction to trigger cell survival, differentiation, proliferation. TKs belong to a large class of enzyme containing receptor tyrosine kinases (RTKs) and non-receptor tyrosine kinases. RTKs are key regulators of cellular processes and are identified to be involved in several pathophysiologies of diseases. So far, twenty subfamilies of RTK have been identified, such as EGFR (Epidermal growth factor receptor), FGFR (Fibroblast growth factor receptor), VEGFR (Vascular endothelial growth factor receptor), RETR (RET receptor), EPHR (Eph receptor), and DDR (Discoidin domain receptor) in humans. RTK molecules contains two regions, including an extracellular ligand-binding region with a single transmembrane helix, and a cytoplasmic region containing a protein tyrosine kinase domain with additional carboxy-(C-)terminal as well as juxtamembrane regulatory regions. Preferably, the TKI according to the disclosure is an inhibitor of vascular endothelial growth factor receptor (VEGFR) including VEGFR1, VEGFR2, and VEGFR3 to inhibit angiogenesis. More preferably, the TKI is Cabozantinib, Regorafenib, Axitinib, Afatinib, Ninetedanib, Crizotinib, Alectinib, Trametinib, Dabrafenib, Sunitinib, Ruxolitinib, Vemurafenib, Sorafenib, Ponatinib, Encorafenib, Brigatinib, Pazopanib, Dasatinib, Imatinib, Lenvatinib, Vandetanib, Surufatinib or Sitravatinib.

It is believed, though not intended to be restricted by any theory, that multi-targeting kinase inhibitors possess a very potent capacity to modulate TME and boost immune response, especially combined with an immune checkpoint inhibitor such as anti-PD-1 or anti-PD-L1 antibody. It achieves a better therapeutic efficacy outcome than PD-1/PD-L1 blockade monotherapy.

In one embodiment, the immune checkpoint inhibitor can be used in combination with the pharmaceutical combination described herein to stimulate an immune response against cancer cells to treat a cancer. Immune checkpoint inhibitors suitable for use in the present disclosure comprise an antagonist of an inhibitory receptor which inhibits the PD-1, CTLA-4, T cell immunoglobulin-3, B and T lymphocyte attenuator, V-domain Ig suppressor of T cell activation or lymphocyte-activation gene 3 pathway, such as anti-PD-1 antibodies or agents, anti-PD-L1 antibodies or agents, anti-CTLA-4 antibodies or agents, anti-TIM-3 (T cell immunoglobulin-3) antibodies or agents, anti-BTLA (B and T lymphocyte attenuator) antibodies or agents, anti-VISTA (V-domain Ig suppressor of T cell activation) antibodies or agents, anti-LAG-3 (lymphocyte-activation gene 3) antibodies or agents, KIR (killer-cell immunoglobulin-like receptor) antibodies or agents, TIM-3 immunoglobulin domain and mucin domain 3) antibodies or agents, A2AR (adenosine A2A receptor inhibitor, CD276 antibodies or agents, and VCTN1 antibodies or agents. Examples of PD-1 or PD-L1 inhibitors include, without limitation, humanized antibodies blocking human PD-1 such as Pembrolizumab (anti-PD-1 Ab, trade name Keytruda) or Pidilizumab (anti-PD-1 Ab), Bavencio® (anti-PD-L1 Ab, Avelumab), Imfinzi® (anti-PD-L1 Ab, Durvalumab), and Tecentriq® (anti-PD-L1 Ab, Atezolizumab), as well as fully human antibodies such as Nivolumab (anti-PD-1 Ab, trade name Opdivo) and cemiplimab-rwlc (anti-PD-1 Ab, trade name Libtayo®). Other PD-1 inhibitors may include presentations of soluble PD-1 ligand including small molecular drugs blocking human PD-1/PD-L1 such as BMS-1166, without limitation, PD-L2 Fc fusion protein also known as B7-DC-Ig or AMP-244 and other PD-1 inhibitors presently under investigation and/or development for use in therapy. In addition, immune checkpoint inhibitors may include, without limitation, humanized or fully human antibodies blocking PD-L1 such as Durvalumab and MIH1 and other PD-L1 inhibitors presently under investigation. In some embodiments, the amount of the immune checkpoint inhibitor ranges from about 0.5% (w/w) to about 15% (w/w), about 0.5% (w/w) to about 10% (w/w), about 0.5% (w/w) to about 5% (w/w), about 1.0% (w/w) to about 20% (w/w), about 1.0% (w/w) to about 15% (w/w), about 1.0% (w/w) to about 10% (w/w) or about 1.0% (w/w) to about 5% (w/w).

In one embodiment, the HDAC inhibitor and TKI are administered with the immune checkpoint inhibitor simultaneously or sequentially in either order or in alternation. In some embodiments of the present disclosure, the HDAC inhibitor, the TKI, and the immune checkpoint inhibitor are administered simultaneously.

In a further embodiment, the method further comprises administering one or more additional anti-cancer agents. The additional anti-cancer agent is any anti-cancer agent described herein or known in the art. In one embodiment, the additional anti-cancer agent is a chemotherapy or a platinum-based doublet chemotherapy. In one embodiment, the additional anti-cancer agent is an anti-VEGF antibody or VEGFR small-molecule inhibitor. In other embodiments, the anti-cancer agent is a platinum agent (e.g., cisplatin, carboplatin), a mitotic inhibitor (e.g., paclitaxel, albumin-bound paclitaxel, docetaxel, taxotere, docecad), a fluorinated Vinca alkaloid (e.g., vinflunine, javlor), vinorelbine, vinblastine, etoposide, or pemetrexed gemcitabin. In one embodiment, the additional anti-cancer agent is 5-flurouracil (5-FU). In certain embodiments, the additional anti-cancer agent is any other anti-cancer agent known in the art.

The pharmaceutical combination of the present invention may be formulated with a “carrier.” As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. For example, the pharmaceutical combinations can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, lotion, gel, ointment, or a controlled-release patch or spray applied to the skin; (4) intravaginally or intrarectally, for example, as a pessary, cream, suppository or foam; (5) sublingually; (6) ocularly; (7) transdermally; (8) transmucosally; or (9) nasally.

In a further aspect, the present invention provides a method of treating a cancer in a subject, the method comprising administering a pharmaceutical combination of the invention to the subject.

In some embodiments, the cancer includes, but is not limited to, melanoma, head and neck cancer, merkel cell carcinoma, hepatocellular carcinoma, renal cell carcinoma, colorectal cancer, endometrial carcinoma, cervical cancer, esophageal squamous cell carcinoma, small cell lung cancer, non-small cell lung cancer, breast cancer, gastric carcinoma, esophagogastric junction carcinoma, classical Hodgkin lymphoma, Non-Hodgkin lymphoma, urothelial carcinoma, primary mediastinal large B-cell lymphoma, glioblastoma, pancreatic cancer, benign prostate hyperplasia, prostate cancer, ovarian cancer, chronic lymphocytic leukemia, Merkel cell carcinoma, acute myeloid leukemia, gallbladder cancer, cholangiocarcinoma, urinary bladder cancer, or uterine cancer.

In some embodiments, the pharmaceutical combination of the invention may be provided in a single formulation or medicament. In other embodiments, the pharmaceutical combination of the invention may be provided in separates formulations or medicaments. A pharmaceutical combination may be formulated in a variety of and/or a plurality of forms adapted to one or more preferred routes of administration. Thus, a pharmaceutical combination can be administered via one or more known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical combination, or a portion thereof, can be administered to a mucosal surface, such as by administration to, for example, the nasal or respiratory mucosa (e.g., by spray or aerosol). A pharmaceutical combination, or a portion thereof, also can be administered via a sustained or delayed release.

A formulation may be conveniently presented in unit dosage form and may be prepared by methods well known in the art of pharmacy. Methods of preparing a combination with a pharmaceutically acceptable carrier include the step of bringing the pharmaceutical combination of the invention into association with a carrier that constitutes one or more accessory ingredients. In general, a formulation may be prepared by uniformly and/or intimately bringing the active compound into association with a liquid carrier, a finely divided solid carrier, or both, and then if necessary, shaping the product into the desired formulations.

The amount of a compound that will be effective in the treatment of a particular disorder or condition, including cancer, will depend on the nature of the disorder or condition, and can be determined by standard clinical techniques. In addition, in vitro assays may optionally be employed to help identify optimal dosage ranges. The precise dose to be employed in the formulation will also depend on the route of administration, and the progression of the disease or disorder, and should be decided according to the judgment of the practitioner and each patient's circumstances. A preferred dosage will be within the range of about 0.01-1000 mg/kg of body weight, about 0.1 mg/kg to 100 mg/kg, about 1 mg/kg to 100 mg/kg, about 10 mg/kg to 75 mg/kg, about 0.1-1 mg/kg, etc. for the combination or each component of the combination.

The present invention is illustrated by the following examples. It is to be understood that the particular examples, materials, amounts, and procedures are to be interpreted broadly in accordance with the scope and spirit of the invention as set forth herein.

### EXAMPLES

### Materials and Methods

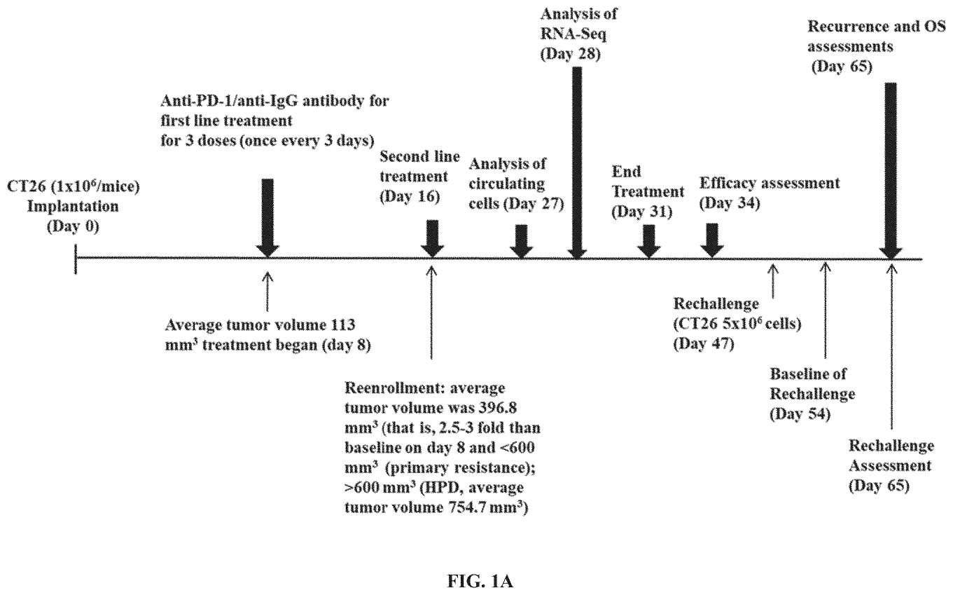

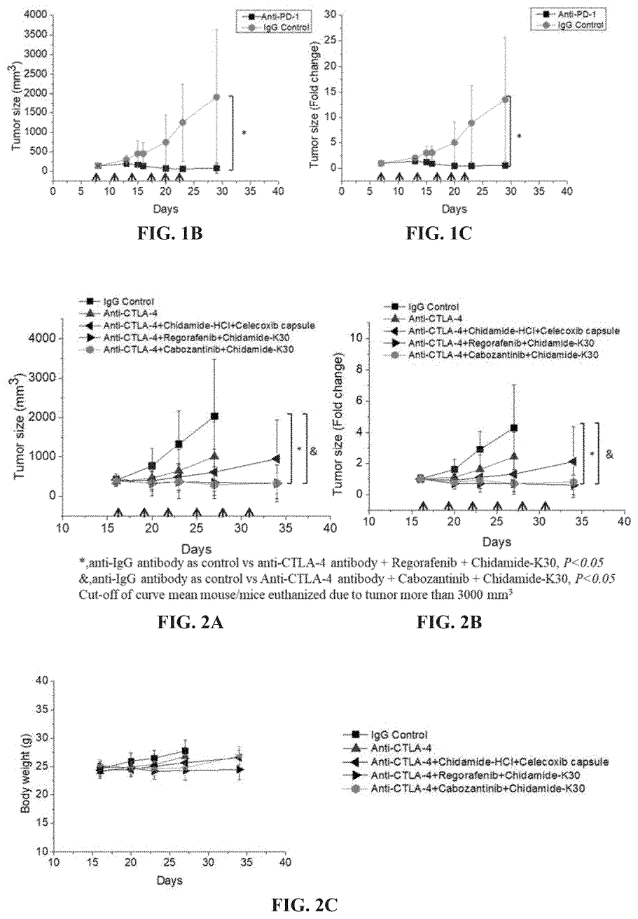

To Overcome the Primary and Acquired Resistance, and HPD to First Line PD-1 Checkpoint Blockade Therapy. Male Balb/c mice bearing subcutaneous CT26 tumors (1×10⁶ cell/mice) were treated with first line of therapy of anti-PD-1 antibody (Purchased from InvivoMab, cat #BE00146) treatment (mean tumor volume: 113 mm³ when treatment began), administered intraperitoneally (i.p.) at 2.5 mg/kg, once every 3 days for 3 doses. When tumors responded to treatment with anti-PD-1 antibody (wherein the tumors were significantly shrunk), then the treatment was continuously given for a further 3 doses (i.e., total 6 doses). If the tumors were shrunk at the beginning of anti-PD-1 antibody treatment (after 3 doses), then they grew gradually as a result of continuous anti-PD-1 antibody treatment (i.e., total 6 doses) due to partially effective in inhibiting tumor growth, and further grew in size to develop acquired resistance. When tumors did not respond at the beginning of treatment of anti-PD-1 antibody (after 3 doses) and met the criteria of 2.5-3 consecutive increases in tumor volume (and tumor volume was <600 mm³), it was considered primary resistance. However, hyperprogressive disease (HPD) has been defined as tumors grew greater than 600 mm³ after 3 doses of first line anti-PD-1 antibody treatment. These mice with primary resistance, acquired resistance and HPD were subsequently reenrolled in a second line of therapy for efficacy study as shown in FIG. 1(A). The second-line therapies were as follows. Anti-IgG antibody as control (Purchased from InvivoMab, cat #BE0089, Bio X Cell), anti-PD-1 antibody (Purchased from InvivoMab, cat #BE0146, Bio X Cell) and anti-CTLA-4 antibody (Purchased from InvivoMab, cat #BE0164, Bio X Cell) were administered intraperitoneally (i.p.) at 2.5 mg/kg, once every 3 days for 6 doses. Anti-CTLA-4 Ab (2.5 mg/kg)+Chidamide-HCl (50 mg/kg)+Celecoxib (50 mg/kg), anti-CTLA-4 Ab (2.5 mg/kg)+Regorafenib (30 mg/kg)+Chidamide-k30 (50 mg/kg) and anti-CTLA-4 Ab (2.5 mg/kg)+Cabozantinib (30 mg/kg)+Chidamide-k30 (50 mg/kg) were administered p.o. every day for 16 times. Tumor diameter was measured every 2-3 days, and tumor volume (in mm³) was calculated using a caliper. The anti-cancer activity was measured from the start of the treatment until the tumor volume reached 3,000 mm³. Tumor volume was calculated as length×width²×0.5.

Anti-Colorectal Cancer Activity in Animal Models. Animal study was approved and overseen by The Taipei Medical University Institutional Animal Care and Use Committee (TMU IACUC, NO: LAC-2020-0103, LAC-2019-0644). Six- to eight-week-old male Balb/c mice (National Laboratory Animal Center, Taiwan) were used for all animal experiments. CT26 cell line was purchased from ATCC. CT26 tumor cell lines were grown in RPMI-1640 supplemented with 10% (vol/vol) FBS at 37° C., 5% CO₂. Tumors were established by s.c. injection of 1×10⁶ CT26 cells with Matrigel (354248, Corning®) into the left flank of mice, and growth determined by measuring two perpendicular diameters. Tumors were allowed to grow for 8-12 days (tumor size about 110-250 mm³) before randomization and treatment. Animals were euthanized when tumors reached more than 3000 mm³ in volume. An anti-IgG antibody (BE0089, Lot #716719J3, Bio X Cell), anti-PD-1 antibody (BE0146, Lot #717918D1, Lot #735019J3, Lot #780120J3, Lot #73501901, Bio X Cell), anti-PD-L1 antibody (BE0101, Lot #720619F1, Bio X Cell) and anti-CTLA-4 antibody (BE0164, Lot #702418A2B, Bio X Cell) were administered i.p. at 2.5 mg/kg twice a week for three weeks. All antibodies were diluted to appropriate concentrations in 100 μL of sterile PBS (pH 7.4, Invitrogen Life Technologies). Axitinib (HY-10065, 30 mg/kg, po daily, MedChemExpress USA), Lenvatinib (HY-10981, 10 mg/kg, po daily, MedChemExpress USA), Olaparib (HY-10162, 50 mg/kg, po daily, MedChemExpress USA), Ibrutinib (HY-10997, 6 mg/kg, po daily, MedChemExpress USA), Cabozantinib (HY-13016, 30 mg/kg, po daily, MedChemExpress USA), Regorafenib (HY-1031, 30 mg/kg, po daily, MedChemExpress USA), RMC-4550 (HY-116009, 30 mg/kg, po daily, MedChemExpress USA), Sitravatinib (HY-16961, 20 mg/kg, po daily, MedChemExpress USA), Entinostat (HY-12163, 20 mg/kg, po q2d, MedChemExpress USA), Vorinostat (HY-10221, 150 mg/kg, po daily, MedChemExpress USA), Chidamide-k30 or Chidamide-HCl salt (50 mg/kg, po daily, produced from GNTbm, Taipei, Taiwan), Celecoxib (50 mg/kg, po daily, capsule/Celebrex®, Pfizer Pharmaceuticals LLC) were given for 16 days. Axitinib, Lenvatinib, Olaparib, Ibrutinib, Cabozantinib, Regorafenib, Entinostat, Vorinostat, RMC-4550, Sitravatinib and Celecoxib were dissolved in DMSO and diluted in PBS before administration. Chidamide-k30 and Chidamide-HCl salt were dissolved in water. Animals were euthanized when tumors reached more than 3000 mm³ in volume. The anti-cancer activity was measured from the start of the treatment until the tumor volume reached 3,000 mm³. Tumor volume was calculated as length×width²×0.5. In this study, we defined Complete Response (CR, <0.5 time tumor growth in the tumor bearing mice at three days after the end of treatment); Partial Response (PR, tumor size ≥0.5 time tumor growth, but <1 times tumor growth in the tumor bearing mice at three days after the end of treatment); Stable Disease (SD, tumor size ≥1 time tumor growth, but <5 times tumor growth in the tumor bearing mice at three days after the end of treatment); Progressive Disease (PD, tumor size ≥5 times tumor growth in tumor bearing mice at three days after the end of treatment) for the evaluation of treatment efficacy. The recurrence was defined as when having tumor growth at least 5 fold in mice with CR or PR response after first tumor assessment.

In order to demonstrate that the invented combination can overcome the drug resistance issues after treatment with anti-PD-1 Ab, all mice were treated with anti-PD-1 Ab first.

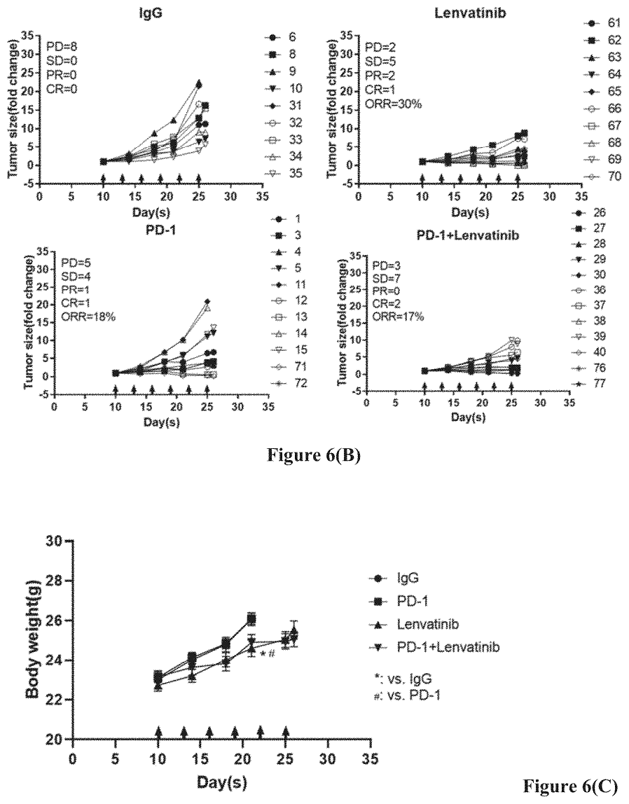

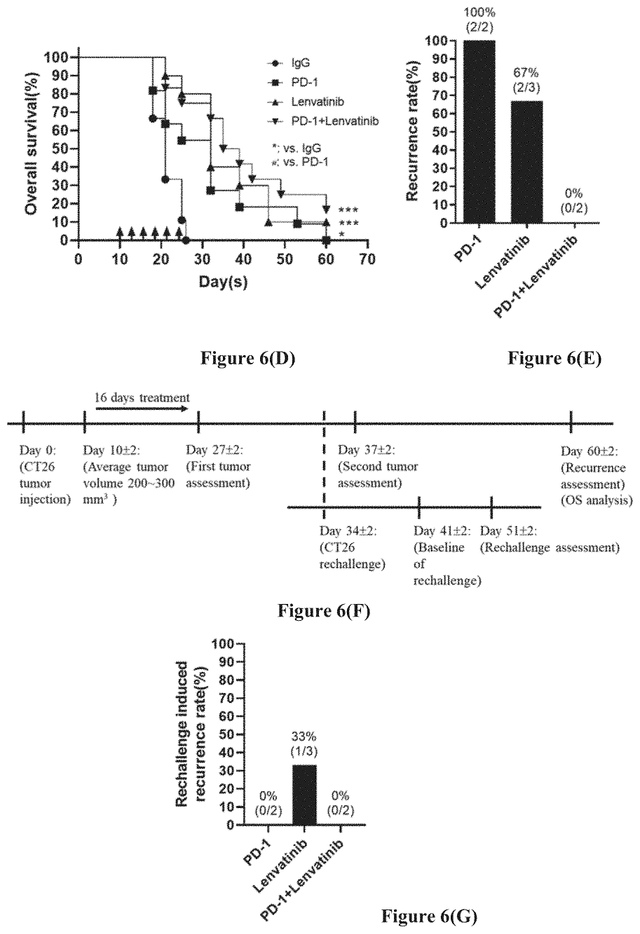

Tumor Rechallenges in Animal Models. All mice with PR/CR response after treatment were rechallenged with CT26 cells on the contralateral side (please see FIG. 6(F)). The rechallenge with CT26 was performed on day 34±2, which was 7 days (day 41±2) after first tumor assessment (day 27±2), with injection of 5×10⁶ CT26 cells per mouse. After rechallenge with CT26 cells, tumors were allowed to grow for another 7 days (day 41±2) to determine the baseline as 1 fold. After further 10 days (day 51±2), the tumor growth was evaluated for the rechallenge. If two of the following criteria are met, the response will be considered as rechallenge-induced recurrence/relapse: first, the tumor size over 2 folds when compared to that of baseline on day 41±2; second, the tumor volume on day 51±2 was over 300 mm³. Relapse happens when immunity is not sufficiently activated. If the tumor growth is inhibited, it means the immunity is activated.

Survival Rate in Animal Models. After tumor assessment the tumor volume of the mice was measured once every three or four days (twice/week). The tumor-bearing mice were regarded as dead when the tumor volume reached 3,000 mm³. All treatment groups were recorded and analyzed.

Flow Cytometry. The following antibodies and reagents were used for flow cytometry: CD8a PerCP-Cy5.5 (53-6.7; BioLegend), CD4 PE (GK 1.5; BioLegend), CD25 PerCP-Cy5.5 (PC61; BioLegend), Foxp3 PE (MF14; BioLegend), CD3 APC (17A2; BioLegend), CD11b APC (M1/70; BioLegend), Ly-6C PerCP-Cy5.5 (HK 1.4; BioLegend), Ly-6G PE (1A8; BioLegend), WIC-11-PE (BM8; BioLegend), CD45 FITC (30-F11; BioLegend). Flow cytometry was performed with a Caliber (BD Biosciences) and the data were analyzed with FACS Diva software (BD Biosciences).

To assess the level of tumor infiltrating lymphocyte in tumors, further assays were performed to analyze the intratumoral CD8⁺, CD4⁺, regulatory T-cell (Treg), PMN-MDSC, M-MDSC, TAM populations. Tumor infiltrating lymphocytes were first purified from tumor samples excised from mice on day 12 after initiation of the Cabozantinib or Regorafenib treatments with or without Chidamide-k30 plus anti-PD-1 Ab. Briefly, primary tumor tissues were harvested, weighed, and minced into fine fragments. Collagenase IV (Sigma-Aldrich) at 1 mg/mL in HBSS (Invitrogen Life Technologies) was added to each sample at a ratio of 1 mL per 200 mg of tumor tissue. Samples were incubated on an end-over-end shaker for 150 min at 37° C. The resulting tissue homogenates were 0.4-μm filtered and washed three times in PBS (BD Biosciences), and then separated via Percoll gradient to isolate mononuclear cells, and 1×10⁶ cells per sample were used for antibody labeling. CD8⁺ T-cell level was assessed using previously established phenotypic criteria of CD45⁺CD3⁺CD8; Treg cell level was assessed using previously established phenotypic criteria of CD45⁺CD3⁺CD25⁺FoxP3⁺; PMN-MDSC and M-MDSC cell levels were assessed using previously established phenotypic criteria of CD45⁺/CD11b⁺/Ly6G⁺/Ly6C⁻ and CD45⁺/CD11b⁺/Ly6G⁻/Ly6C⁺, respectively; TAM cell level was assessed using previously established phenotypic criteria of CD45⁺CD11b⁺CHM-11⁺Ly6C⁺, and total mononuclear cells were used as a common denominator.

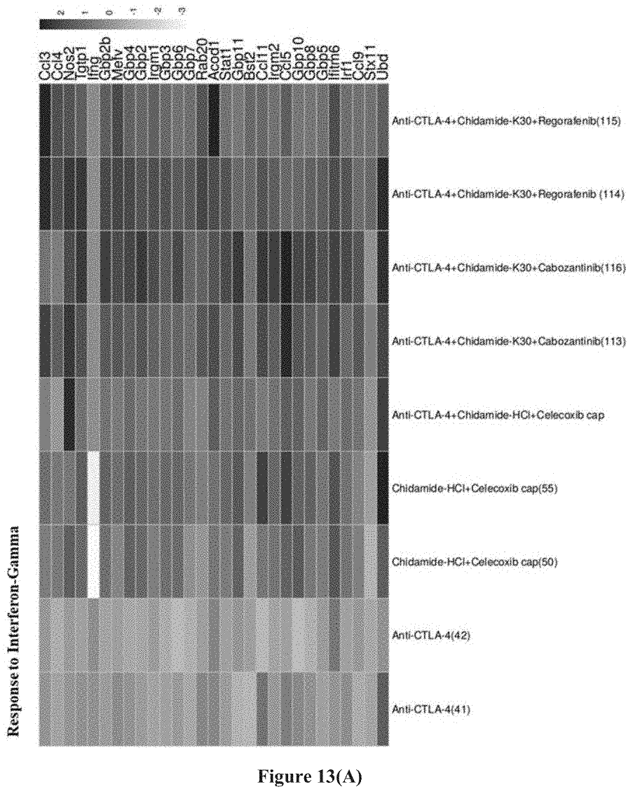

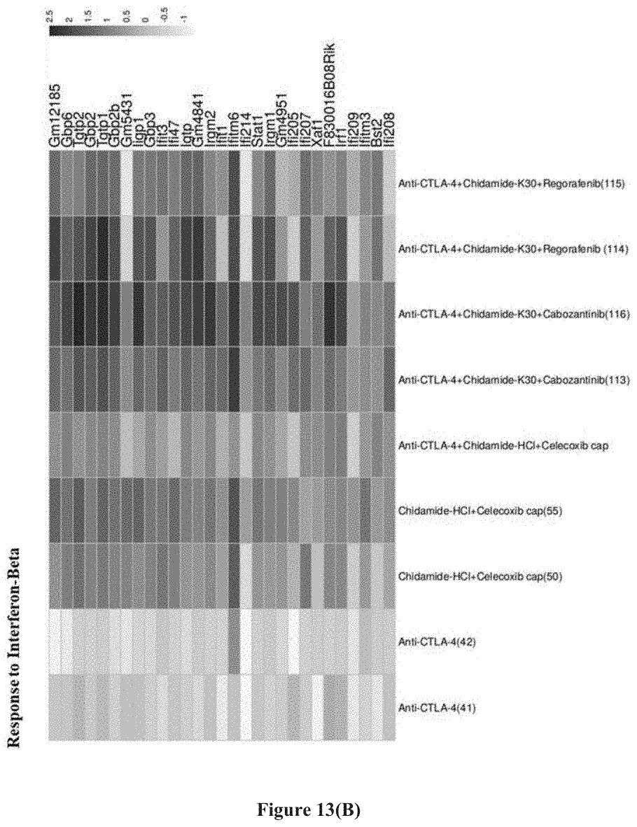

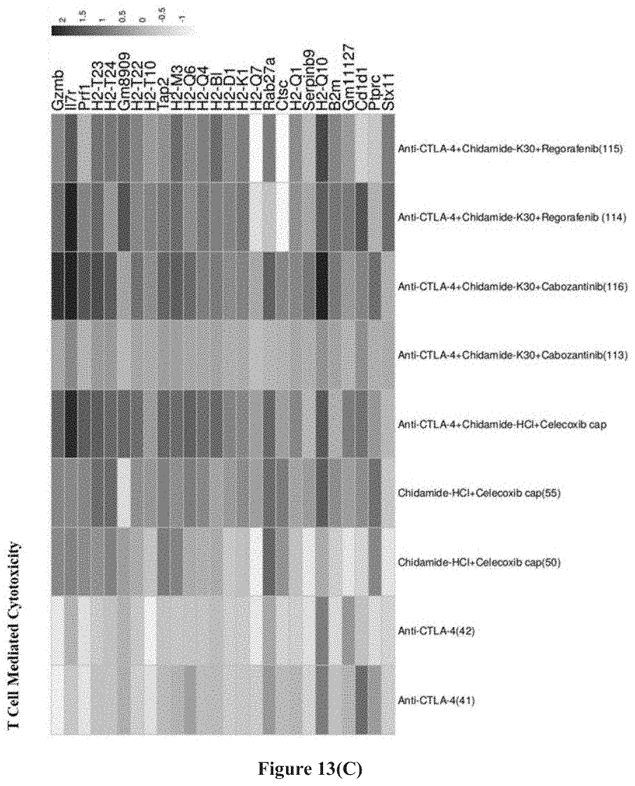

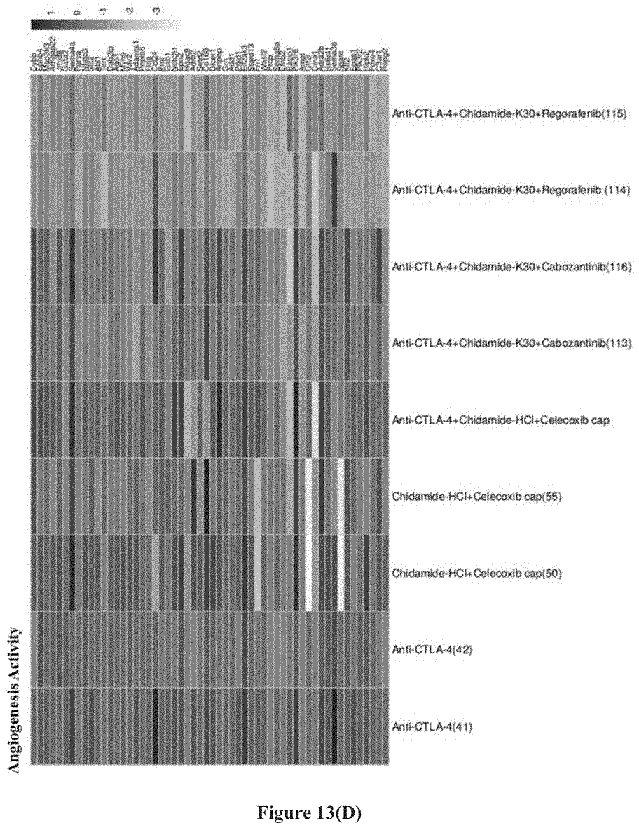

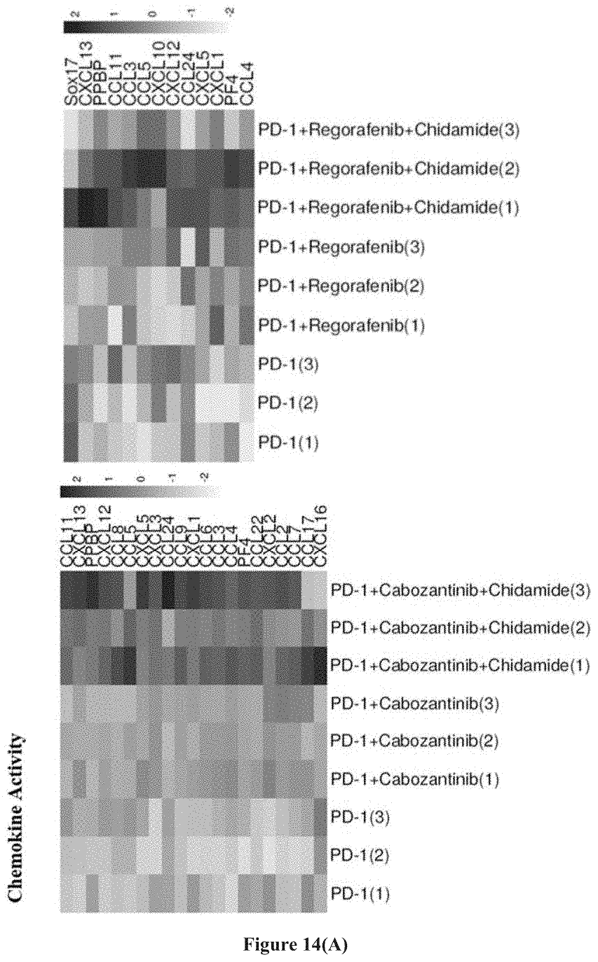

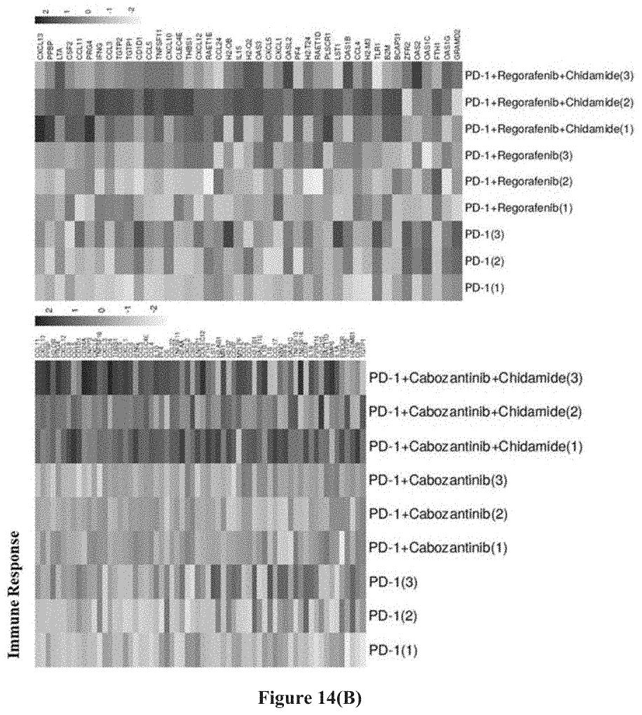

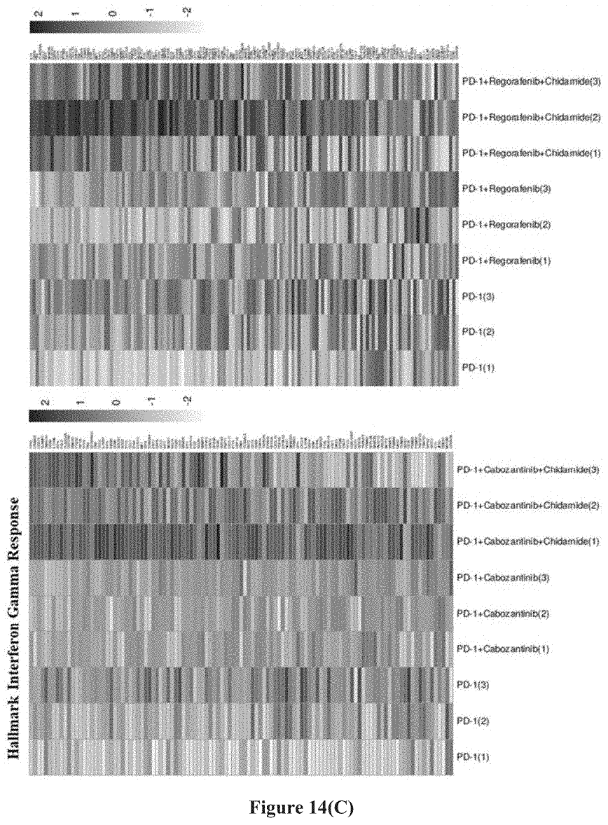

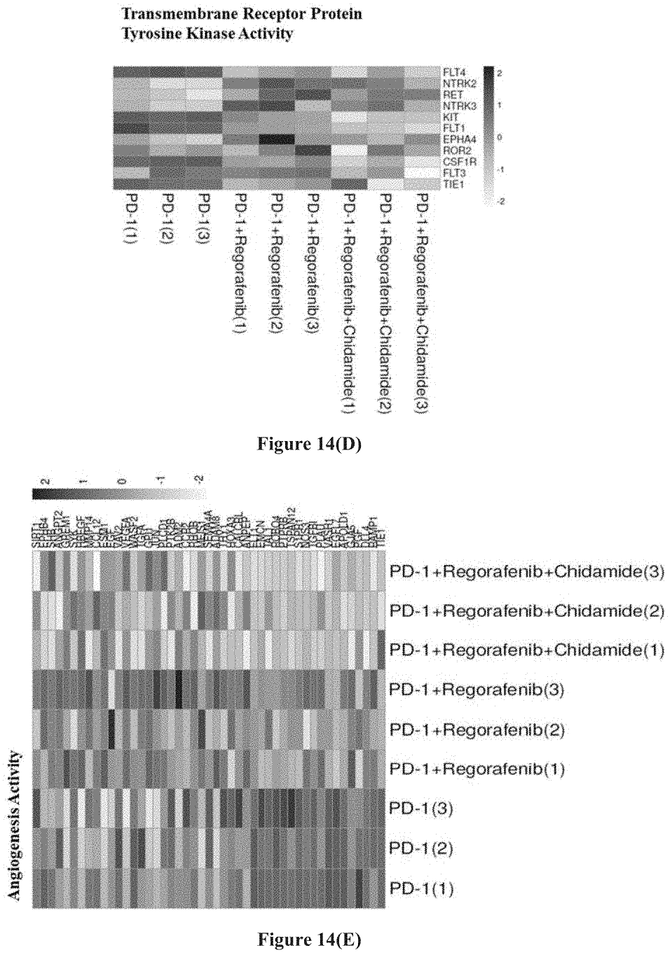

RNA Quantification and Qualification. The drug-resistant mice after first-line anti-PD-1 Ab therapy were randomized and treated with different regimens, and the tumors were excised and collected on day 13 after starting second line treatment. The naïve CT26 tumor-bearing mice were randomized and treated with different regimens, and the tumors were excised and collected on day 9 after starting treatment. All tumor samples were snap-frozen in liquid nitrogen, and samples were then homogenized in Trizol (Invitrogen Life Technologies). RNA Purity and quantification were checked using SimpliNano™-Biochrom Spectrophotometers (Biochrom, MA, USA). RNA degradation and integrity were monitored by Qsep 100 DNA/RNA Analyzer (BiOptic Inc., Taiwan). The results are shown in FIGS. 13 and 14.

Library Preparation for Transcriptome Sequencing. A total amount of 1 μg total RNA per sample was used as input material for the RNA sample preparations. Sequencing libraries were generated using KAPA mRNA HyperPrep Kit (KAPA Biosystems, Roche, Basel, Switzerland) following the manufacturer's recommendations, and index codes were added to attribute sequences to each sample. PCR products were purified using KAPA Pure Beads system, and the library quality was assessed on the Qsep 100 DNA/RNA Analyzer (BiOptic Inc., Taiwan).

Bioinformatics. The original data obtained by high-throughput sequencing (Illumina NovaSeq 6000 platform) were transformed into raw sequenced reads by CASAVA base calling and stored in FASTQ format. FastQC and MultiQC were used to check fastq files for quality. The obtained raw paired-end reads were filtered by Trimmomatic (v0.38) to discard low-quality reads, trim adaptor sequences, and eliminate poor-quality bases with the following parameters: LEADING: 3 TRAILING: 3 SLIDINGWINDOW: 4:15 MINLEN: 30. The obtained high-quality data (clean reads) was used for subsequent analysis. Read pairs from each sample were aligned to the reference genome by the HISAT2 software (v2.1.0). FeatureCounts (v1.6.0) was used to count the reads numbers mapped to individual genes. For gene expression, the “Trimmed Mean of M-values” normalization (TMM) was performed DEGseq (v1.36.1) without biological duplicate and the “Relative Log Expression” normalization (RLE) was performed using DESeq2 (v1.22.1) with biological duplicate. Differentially expressed genes (DEGs) analysis of two conditions was performed in R using DEGseq (without biological replicate) and DESeq2 (with biological replicate), which is based on negative binomial distribution and Poisson distribution models, respectively. The resulting p-values were adjusted using the Benjamini and Hochberg's approach for controlling the FDR. GO and KEGG pathway enrichment analysis of DEGs were conducted using clusterProfiler (v3.10.1). Gene set enrichment analysis (GSEA) was performed with 1,000 permutations to identify enriched biological functions and activated pathways from the molecular signatures database (MSigDB). MSigDB is a collection of annotated gene sets for use with GSEA software, including hallmark gene sets, positional gene sets, curated gene sets, motif gene sets, computational gene sets, GO gene sets, oncogenic gene sets, and immunologic gene sets. In addition, Weighted Gene Co-expression Network Analysis (WGCNA) was constructed by the co-expression network based on the correlation coefficient of expression pattern using the WGCNA (v1.64) package in R.

### Example 1: To Overcome the Resistance from First Line Anti-PD-1 Ab Treatment by Tyrosine Kinase Inhibitors Plus HDAC Inhibitor Combined with Anti-CTLA-4 Antibody in CT26-Bearing Mice

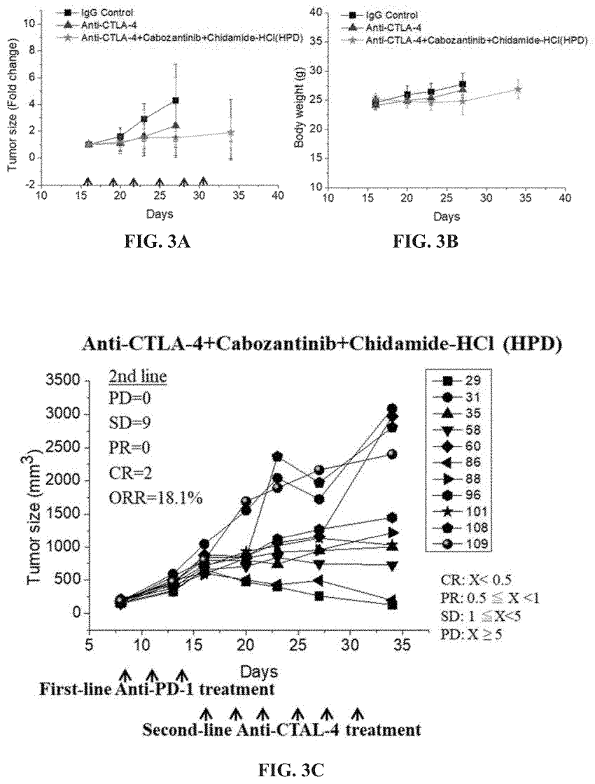

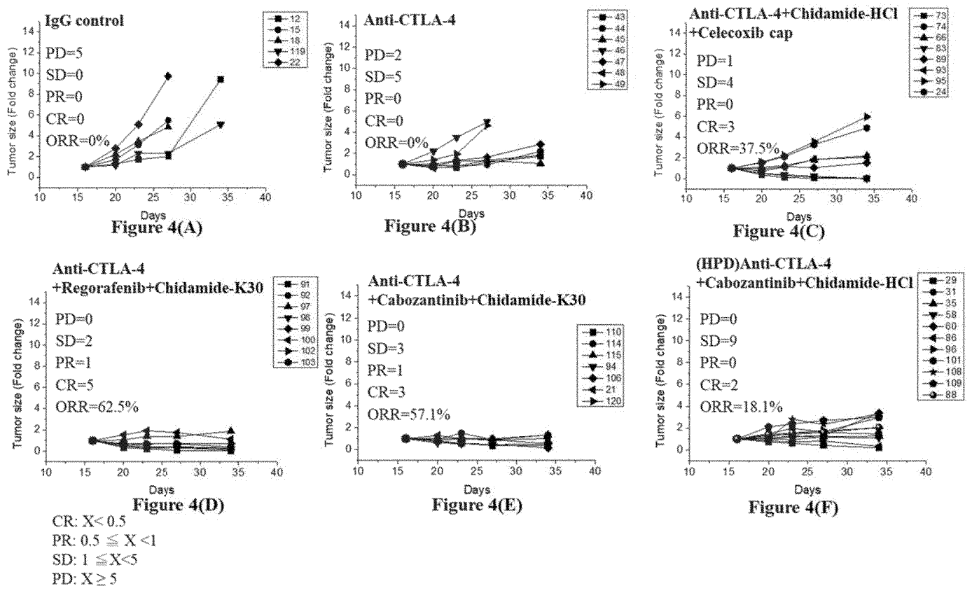

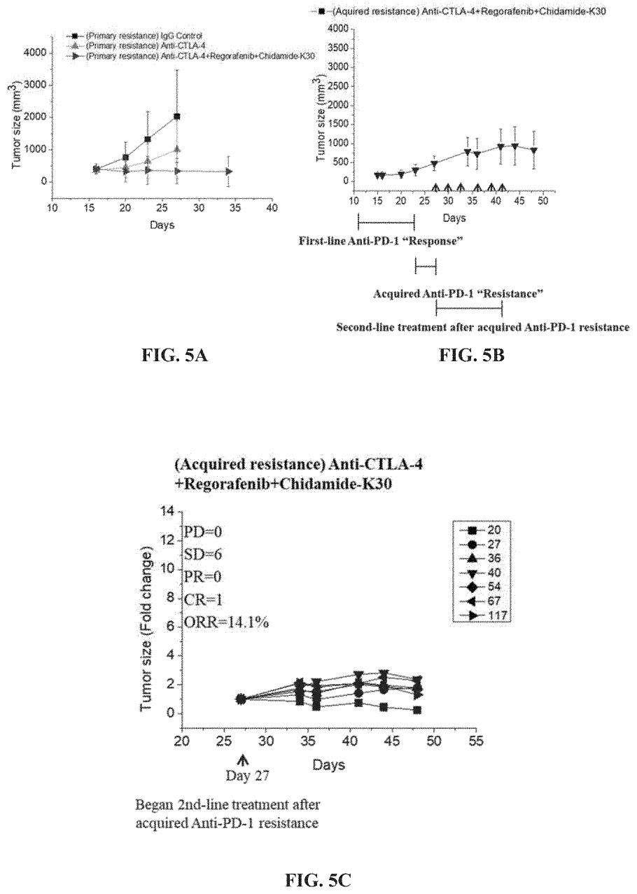

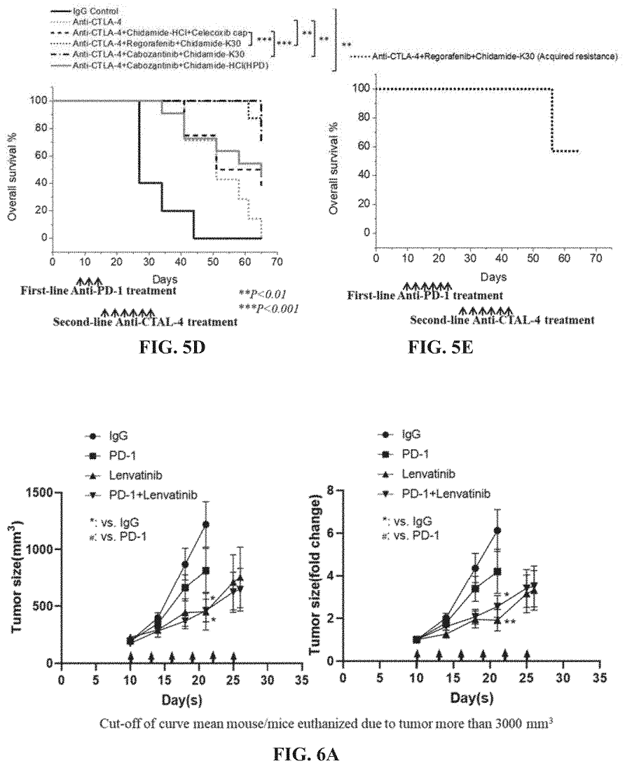

In this example, the mice were treated with second line therapy to mimic the treatment for first line drug resistance occurring in human first line cancer therapy—in which a great portion of human cancer patients receiving first line anti-PD-1 antibody therapy will develop resistance, including primary and acquired resistance or HPD (hyperprogressive disease)—for the evaluation of the anti-cancer potency of second line therapy with tyrosine kinase inhibitors plus HDAC inhibitors combined with anti-CTLA-4 antibody when first line anti-PD-1 antibody therapy has failed. To evaluate the effectiveness of different treatments for first line anti-PD-1 antibody drug resistance, the platform with treatment schedule was designed as outlined in FIG. 1(A). As shown in FIG. 1, first, 110 mice were treated with anti-PD-1 antibody as first line treatment, and 10 mice were treated with anti-IgG antibody as a negative control. Eighteen of the 110 mice achieved response by first line anti-PD-1 antibody treatment, wherein the objective response rate (ORR) of 16.4% (18/110) was achieved. As shown in FIG. 1(B) and (C), the tumor volume in the mice responsive to first line anti-PD-1 Ab treatment was significantly inhibited in comparison with the anti-IgG Ab group in the CT26 tumor-bearing mice model. Eighty five of the 110 mice showed primary resistance to first line anti-PD-1 antibody treatment, with occurrence rate of 77.3% (85/110). Furthermore, the acquired resistance to treatment with first line anti-PD-1 antibody treatment developed gradually and eventually relapsed with occurrence rate of 28% (7/25), as shown in Table 1. These results suggested that primary resistance to treatment with first-line anti-PD-1 antibody was a very challenging issue for cancer immunotherapy. We were interested in whether TKIs plus HDAC inhibitors could improve the ICIs sensitivity through the regulation of TME. For the study, tumors were allowed to grow for 8 days (tumor size average about 113 mm³) before first line treatment with anti-PD-1 antibody (2.5 mg/kg; Lot #735019O1) administered by LP. for three doses (one dose every 3 days). In mice responsive to first line anti-PD-1 antibody treatment, the tumors were inhibited or sustained, that is, the tumors continued to shrink and achieve a CR or PR response with an ORR of 16.4%. However, when tumors met the treatment failure criteria: (1) consecutive increase 2.5 to 3 folds in tumor volume by day 16 (tumor size average 396.8 mm³) after the three doses of first line anti-PD-1 antibody treatment and (2) the tumor volumes were □600 mm³, the mice were defined to develop primary resistance and were reenrolled for second line treatment. These mice with primary resistance to anti-PD-1 Ab therapy were further randomized. With respect to the HPD mice (with incidence of about 10%, 11/110), defined as having hyperprogressive disease wherein tumor volumes were >600 mm³, the average tumor volume was 754.7 mm³ as shown in FIG. 1(A). There were nine different treatment groups (n=9-10 mice/group) as indicated. The mice with primary resistance were randomized into five different second line treatment groups, including anti-IgG Ab (2.5 mg/kg; Lot #716719J3), anti-CTLA-4 Ab (2.5 mg/kg; Lot #702418A2B), anti-CTLA-4 Ab combined with Chidamide-HCl salt (50 mg/kg) plus Celecoxib as positive control, anti-CTLA-4 Ab combined with Regorafenib (30 mg/kg) plus Chidamide-k30 (50 mg/kg), and anti-CTLA-4 Ab combined with Cabozantinib (30 mg/kg) plus Chidamide-k30 (50 mg/kg) groups. Anti-CTLA-4 antibody was administered intraperitoneally (i.p.) for six doses (one dose every 3 days). Chidamide-k30, Chidamide-HCl salt, Regorafenib and Cabozantinib were given by oral administration once daily for 16 days. As shown in FIGS. 2(A) & 2(B), both groups of anti-CTLA-4 Ab combined with Regorafenib plus Chidamide-k30, and anti-CTLA-4 Ab combined with Cabozantinib plus Chidamide-k30 group were more potent to inhibit tumor growth than positive control of anti-CTLA-4 Ab combined with Chidamide-HCl salt plus Celecoxib group and anti-CTLA-4 Ab alone group. These results suggested that anti-CTLA-4 Ab combined with Regorafenib or Cabozantinib plus Chidamide-k30 possessed very potent regulation of TME activity to overcome primary resistance to first line treatment of anti-PD-1 Ab. This result also suggested that anti-CTLA-4 Ab combined with Chidamide-k30 plus Regorafenib or Cabozantinib regimen was more powerful to overcome primary resistance than anti-CTLA-4 Ab combined with Chidamide-HCl salt plus Celecoxib regimen. As shown in FIG. 2(C), the mice in each treatment group did not have significant body weight loss. The HPD mice were treated with anti-CTLA-4 Ab (2.5 mg/kg) combined with Cabozantinib (30 mg/kg) plus Chidamide-HCl salt (50 mg/kg) for 16 days and the large tumors surprisingly showed growth inhibition and some tumors were controlled by continuous shrinking under this treatment regimen as shown in FIG. 3(A)˜3(C). The result suggested that anti-CTLA-4 Ab combined with Chidamide-HCl salt plus Cabozatinib was a very potent regimen to rescue HPD. Our previous studies have proved that Chidamide-HCl salt was more powerful to modulate the TME than Chidamide-k30. This result also suggested that faster tumor growth in HPD mice compared to primary resistance mice required treatment with anti-CTLA-4 Ab combined with Cabozantinib plus Chidamide-HCl salt for significant suppression of tumor growth through immune regulation in the TME to boost immune response. The individual result of each drug-resistant mouse was also shown in FIG. 4. In the anti-IgG Ab group, 5 mice achieved PD with fast tumor growth. Treatment with anti-CTLA-4 Ab achieved 5 mice of SD and 2 mice of PD compared with anti-IgG Ab. The treatment with anti-CTLA-4 Ab combined with Chidamide-HCl salt plus Celecoxib as positive control showed that 3 mice achieved CR and 4 mice achieved SD, and 1 mouse achieved PD (response rate 37.5%). However, the treatment with anti-CTLA-4 Ab combined with Regorafenib plus Chidamide-k30 showed that 5 mice achieved CR, 1 mouse achieved PR, and only 2 mice achieved SD (response rate 62.5%). When anti-CTLA-4 Ab combined with Cabozantinib plus Chidamide-k30, the result demonstrated that 3 mice achieved CR, 1 mouse achieved PR, and 3 mice achieved SD (response rate 57.1%). However, the treatment for HPD mice with anti-CTLA-4 Ab combined with Cabozantinib plus Chidamide-HCl salt showed that 2 mice achieved CR and 9 mice achieved SD (response rate 18.1%). Although the immune response rates for HPD mice were not very high, most tumors were inhibited from growing after treatment with this second line regimen. This result was intriguing because HPD tumors are very difficult to be effectively reduced and inhibited. With respect to the occurrence of acquired resistance, it was observed that initial first line anti-PD-1 Ab treatment had an effective response on CT26-bearing mice, but after continuous treatment with anti-PD-1 Ab, it did not effectively inhibit tumor growth. This phenomenon is defined as acquired resistance to anti-PD-1 Ab treatment. We were interested in evaluating the therapeutic effect of anti-CTLA-4 Ab combined with Regorafenib plus Chidamide-k30 in mice with acquired resistance to anti-PD1 Ab as shown in FIG. 5. The treatment with regimen anti-CTLA-4 Ab combined with Regorafenib plus Chidamide-k30 effectively inhibited the tumor growth in mice with primary resistance to anti-PD-1 Ab (as shown in FIG. 5(A) & FIG. 2(A)), however the same regimen demonstrated significant suppression of tumor growth in mice with acquired resistance to anti-PD-1 Ab treatment as shown in FIG. 5(B), showing that 1 mice achieved CR, and 6 mice achieved SD (response rate 14.1%) as shown in FIG. 5(C). Furthermore, we were interested in evaluating the survival rate in mice with primary, acquired resistance or HPD to anti-PD-1 Ab treatment. As shown in FIG. 5(D), for mice with primary resistance, anti-CTLA-4 Ab (2.5 mg/kg) combined with Regorafenib (30 mg/kg) plus Chidamide-k30 (50 mg/kg) group achieved an overall survival rate 87.5%; anti-CTLA-4 Ab (2.5 mg/kg) combined with Cabozantinib (30 mg/kg) plus Chidamide-k30 (50 mg/kg) group achieved overall survival rate 71.4%; anti-CTLA-4 Ab (2.5 mg/kg) combined with Chidamide-HCl salt (50 mg/kg) plus Celecoxib (50 mg/kg) group as positive control achieved an overall survival rate 37.5%. This result suggested that these two groups were very powerful in prolonging survival rate as compared with the positive control group. Regarding the HPD mice, treatment with anti-CTLA-4 Ab (2.5 mg/kg) combined with Cabozantinib (30 mg/kg) plus Chidamide-HCl salt (50 mg/kg) achieved a very good overall survival rate of 45.4%. This result was noteworthy because the HPD mice only achieved ORR 18.1%, but achieved an overall survival rate of 45.4% due to significant suppression of tumor growth. The result raised the possibility that the regimen may have had strong modulation capacity of TME resulting in continuous tumor suppression, which would sustain even one month after the end of treatment. Similar results also occurred in treatment groups of anti-CTLA-4 Ab combined with Regorafenib/Cabozantinib plus Chidamide-k30 regimens, wherein the overall survival rate was better than ORR. As shown in FIG. 5(E), the mice with acquired resistance achieved an overall survival rate of 57.1% after treatment with anti-CTLA-4 Ab (2.5 mg/kg) combined with Regorafenib (30 mg/kg) plus Chidamide-k30 (50 mg/kg). This is similar to the survival rate of HPD mice. These results fully demonstrated that anti-CTLA-4 Ab combined with Regorafenib/Cabozantinib plus Chidamide-HCl salt/Chidamide-k30 possessed very potent activity to modulate TME and significantly boost the immune response to overcome the resistance to first-line anti-PD-1 Ab treatment. As shown in Table 2, the initial ORR assessments were performed 3 days after the last drug administration, however the second ORR assessments were additionally scheduled 10 days after the last drug administration due to the observation of continuous tumor shrinkage. Treatment with anti-CTLA-4 Ab combined with Regorafenib plus Chidamide-k30 group was significant in boosting ORR from 62.5% to 87.5% and augmenting CR from 5 to 7 mice in the second assessment in the mice with primary resistance to first-line anti-PD-1 Ab therapy. A similar phenomenon was also shown in anti-CTLA-4 Ab combined with Cabozantinib plus Chidamide-k30 group: it significantly augmented the ORR from 57.1% to 100% and boosted the CR/PR from 4 to 7 mice in the second assessment in the mice with primary resistance to first-line anti-PD-1 Ab therapy. Regarding the HPD mice, the treatment with anti-CTLA-4 Ab combined with Cabozantinib plus Chidamide-HCl salt significantly augmented the ORR from 18.1% to 45.4% in the second assessment. Finally, the treatment with anti-CTLA-4 Ab combined with Regorafenib plus Chidamide-k30 for the mice with acquired resistance to first-line anti-PD-1 Ab therapy also demonstrated increased anti-cancer activity with ORR from 14.2% to 28.5% in the second assessment. Taken together, all these results suggest that the regimen of anti-CTLA-4 Ab combined with Regorafenib/Cabozantinib plus Chidamide-HCl salt/Chidamide-k30 was very powerful to overcome primary, acquired resistance and HPD to first line anti-PD-1 Ab treatment.

TABLE 1
One hundred and twenty male Balb/c mice bearing subcutaneous CT26
tumors were treated with first line of therapy of anti-PD-1 and anti-IgG
(as negative control) antibody (2.5 mg/kg) once every 3 days for 3 doses.

| The | Whether there is a | Types of drug resistance |
|---|---|---|
| number | response to first line anti- | to first-line anti-PD-1 |
| of mice | PD-1 antibody therapy | antibody therapy |
| 10 | Treatment with anti-IgG antibody | N/A |
| | (as negative control) | |
| 18 | Yes | Response* |
| 7 | Initially there was a response and then | Acquired resistance** |
| | there was obvious tumor growth | |
| 85 | NO | Primary resistance*** |

*Response rate (CR% plus PR%): 18/110, 16.4%;
**Acquired resistance rate: 7/25, 28.0%;
***Primary resistance rate: 85/110, 77.3%.

TABLE 2
The response rates after treated with different second line regimens in CT26-bearing
mice with primary, acquired resistance or HPD to first line anti-PD-1 Ab treatment.

| | | Initial | | | | | | | | | | | | | |
|---|---|---|---|---|---|---|---|---|---|---|---|---|---|---|---|
| | | tumor | | | | | | | | | | | Survival | | |
| | | volume | | | | | | | | | | | Rate | | |
| Resistance | Regimens | (mm³) | ORR (%) | PD | SD | PR | CR | ORR (%)^{&} | PD^{&} | SD^{&} | PR^{&} | CR^{&} | (%) | Relapse* | Immunity ^{#} |
| Primary | Anti-IgG Ab | 396 | 0% | 4 | 1 | 0 | 0 | 0% | 5 | 0 | 0 | 0 | 0% | — | — |
| resistance | as control | | | | | | | | | | | | (0/5) | | |
| to first- | Anti-CTLA- | | 0% | 0 | 7 | 0 | 0 | 0% | 2 | 5 | 0 | 0 | 0% | — | — |
| line | 4 Ab | | | | | | | | | | | | (0/7) | | |
| anti-PD- | Anti-CTLA- | | 37.5% | 1 | 4 | 0 | 3 | 37.5% | 2 | 3 | 0 | 3 | 37.5% | 0% | 100% |
| 1 Ab | 4 Ab + | | | | | | | | | | | | (3/8) | (0/3) | (3/3) |
| therapy | Chidamide- | | | | | | | | | | | | | | |
| | HCl salt + | | | | | | | | | | | | | | |
| | Celecoxib | | | | | | | | | | | | | | |
| | Anti-CTLA- | | 62.5% | 0 | 2 | 1 | 5 | 87.5% | 0 | 1 | 0 | 7 | 87.5% | 0% | 100% |
| | 4 Ab + | | | | | | | | | | | | (7/8) | (0/6) | (6/6) |
| | Regorafenib + | | | | | | | | | | | | | | |
| | Chidamide-k30 | | | | | | | | | | | | | | |
| | Anti-CTLA- | | 57.1% | 0 | 3 | 1 | 3 | 100% | 0 | 0 | 3 | 4 | 71.4% | 0% | 100% |
| | 4 Ab + | | | | | | | | | | | | (5/7) | (0/4) | (4/4) |
| | Cabozantinib + | | | | | | | | | | | | | | |
| | Chidamide-k30 | | | | | | | | | | | | | | |
| Hyper- | Anti-CTLA- | 669 | 18.1% | 0 | 9 | 0 | 2 | 45.4% | 3 | 3 | 3 | 2 | 45.4% | 0% | 100% |
| progressive | 4 Ab + | | | | | | | | | | | | (5/11) | (0/2) | (2/2) |
| disease | Cabozantinib + | | | | | | | | | | | | | | |
| (HPD) | Chidamide-HCl | | | | | | | | | | | | | | |
| to first | salt | | | | | | | | | | | | | | |
| line | | | | | | | | | | | | | | | |
| anti-PD- | | | | | | | | | | | | | | | |
| 1 Ab | | | | | | | | | | | | | | | |
| therapy | | | | | | | | | | | | | | | |
| Acquired | Anti-CTLA- | 477 | 14.2% | 0 | 6 | 0 | 1 | 28.5% | 0 | 5 | 1 | 1 | 57.1% | 0% | — |
| resistance | 4 Ab + | | | | | | | | | | | | (4/7) | (0/1) | |
| to first | Regorafenib + | | | | | | | | | | | | | | |
| line | Chidamide-k30 | | | | | | | | | | | | | | |
| anti-PD- | | | | | | | | | | | | | | | |
| 1 Ab | | | | | | | | | | | | | | | |
| therapy | | | | | | | | | | | | | | | |

*The relapse/recurrence was defined as when having tumor growth at least 5 fold in mice with CR or PR response after first tumor assessment.
^{&}The second tumor assessment 10 days after the last administration of second line treatment.
^{#} Mice resistant to CT26 re-challenge.
— Not tested
Response evaluation criteria: fold change of tumor size compared to baseline
PD: x □ 5;
SD: 1 □ x < 5;
PR: 0.5 □ x < 1;
CR: x < 0.5

### Example 2: To Investigate the Anti-Cancer Effect of TM Lenvatinib Combined with Anti-PD-1 Ab in CT26-Bearing Mice

Several reports had indicated that Lenvatinib possessed potent immune modulatory properties that could boost the anti-PD-1 Ab immune response rate in tumor-bearing mice models. We were interested in researching more powerful regimens to regulate the TME for boosting the immune response rate. First, we were to evaluate the Lenvatinib and Lenvatinib combined with anti-PD-1 Ab in CT26-bearing mice model. As shown in FIGS. 6(A) and 6(B), Lenvatinib (10 mg/kg) combined with or without anti-PD-1 Ab (2.5 mg/kg) more significantly suppressed tumor growth than anti-PD-1 Ab (2.5 mg/kg) treatment alone. Individual tumor assessment showed that in anti-PD-1 Ab group 1 mouse achieved CR, 1 mouse achieved PR, 4 mice achieved SD, and 5 mice achieved PD (response rate 18%). Lenvatinib group showed that 1 mouse achieved CR, 2 mouse achieved PR, 5 mice achieved SD, and 2 mice achieved PD (response rate 30%). Anti-PD-1 Ab combined with Lenvatinib group showed that 2 mice achieved CR, 7 mice achieved SD, and 3 mice achieved PD (response rate 17%). Although in the Anti-PD-1 Ab combined with Lenvatinib group, the immune response rate appeared to be low, but 8 mice showed significant suppression of tumor growth as shown in FIG. 6(B). The body weight was mildly reduced in the treatment groups Lenvatinib alone and Lenvatinib combined with anti-PD-1 Ab in comparison with anti-IgG or anti-PD-1 Ab group as shown in FIG. 6(C). Furthermore, the survival rate was analyzed as shown in FIG. 6(D). CT26 tumor-bearing mice were euthanized when tumor volume reached 3000 mm³ after tumor implantation. Regimen anti-PD-1 Ab combined with Lenvatinib or Lenvatinib alone was more powerful in prolonging the survival rate in comparison with the anti-PD-1 Ab group. As shown in FIG. 6(E), the recurrence rate showed that anti-PD-1 Ab combined with Lenvatinib possessed more power to activate the immune system to avoid relapse than Lenvatinib or anti-PD-1 Ab treatment alone. Next, we were interested to evaluate immunity stimulated by different treatments in a rechallenge experiment as outlined in FIG. 6(F). The mice with CR or PR went into a wash-out stage of 7 days (until day 34±2) without any further treatment. Then rechallenge was performed with the same kind of cancer cells (CT26; 5×10⁶) inoculated on the opposite flank for about another 7 days (day 41±2) and then the tumor volume would be determined as baseline (1 fold). The rechallenge tumor was allowed to grow for 10 days, and then assessed to evaluate the tumor growth (day 51±2). The immunity was defined as negative, when it met two conditions: the tumor volume was over 300 mm³, or the tumor size was over 2 fold when compared to baseline. If the immune memory was activated after treatment, the immunity was active and specific to the recognition of the cancer cells with the same antigen and the growth of tumors inoculated during the rechallenge would be inhibited, therefore the immunity was defined as positive. If the immune memory was not induced or not fully activated, resulting in the growth of tumors inoculated during the rechallenge (tumor recurrence), then the immunity would be defined as negative. As shown in FIG. 6(G), in the anti-PD-1 Ab group the 2 mice that achieved PR and CR showed 0% tumor recurrence after the rechallenge. The result demonstrated that these PR and CR mice achieved 100% overall immune memory. In the Lenvatinib combined with anti-PD-1 Ab group, the 2 mice that achieved CR showed 0% tumor recurrence after the rechallenge. It also demonstrated 100% overall immune memory in these mice with CR or PR. In the Lenvatinib alone group, the 3 mice that achieved CR/PR showed 33% tumor recurrence after the rechallenge. It demonstrated 67% overall immune memory. The rechallenge experiment was used to re-confirm whether the regimens have stimulated the immune system's ability to activate immune memory directly or indirectly.

### Example 3: To Investigate the Anti-Cancer Effect of Tyrosine Kinase Inhibitors (TKIs) Combined with Anti-PD-1 Ab in CT26-Bearing Mice

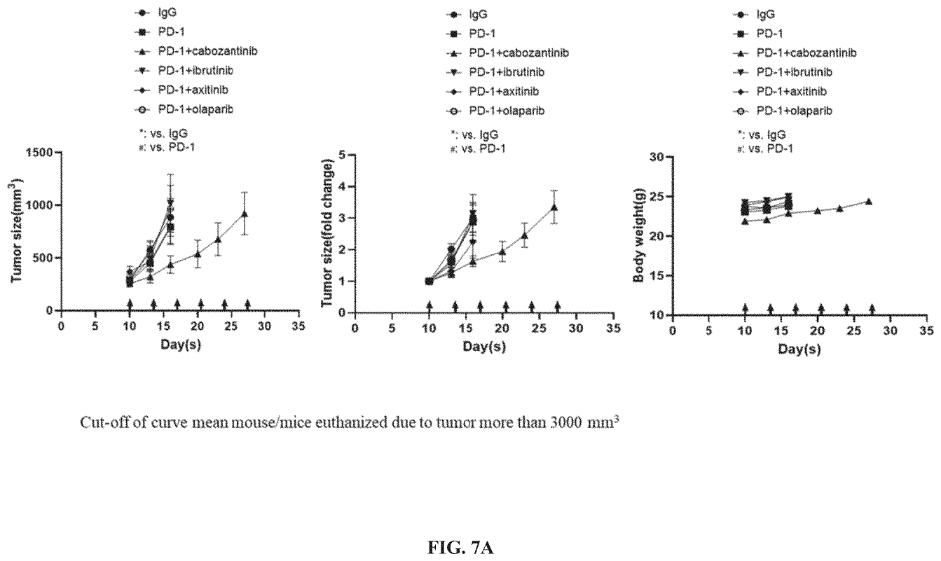

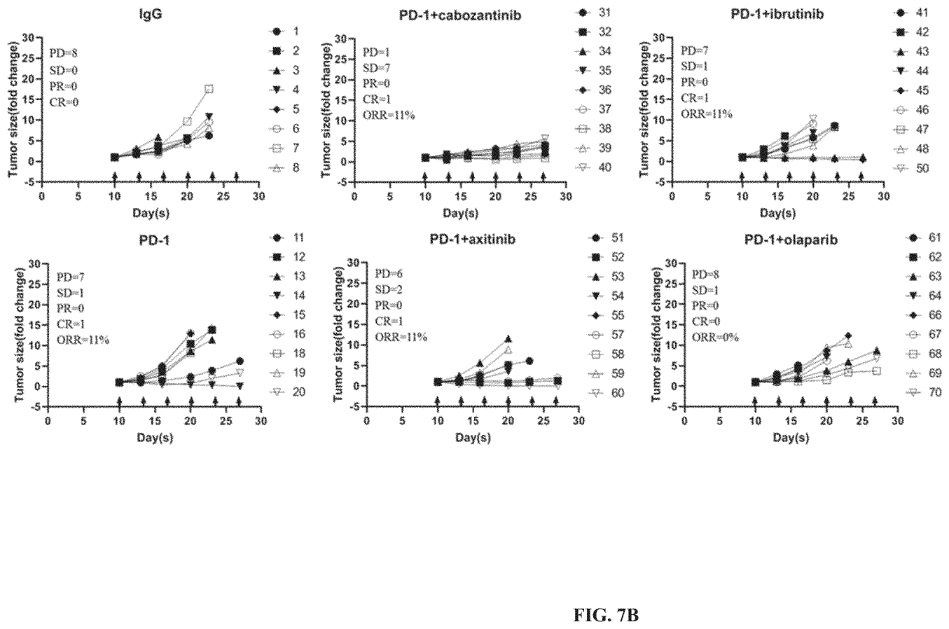

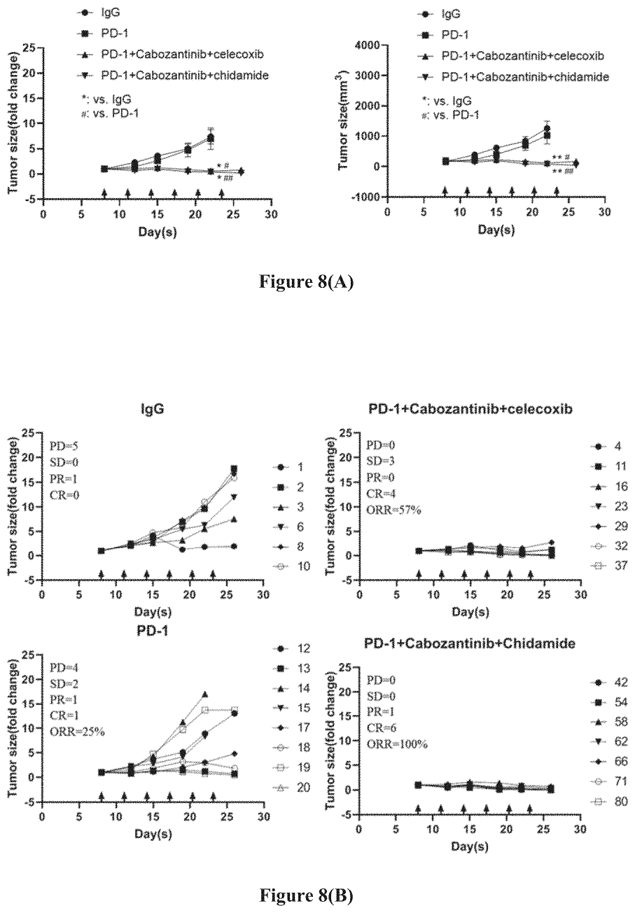

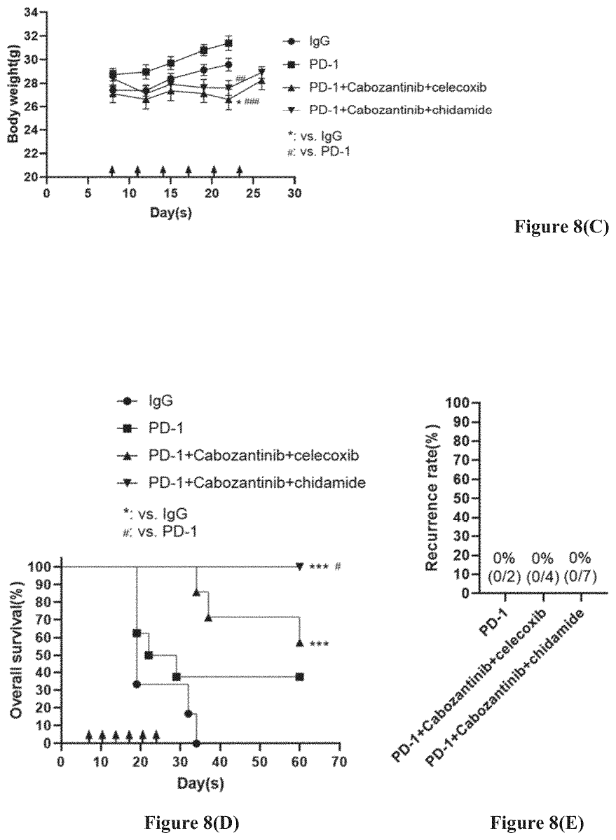

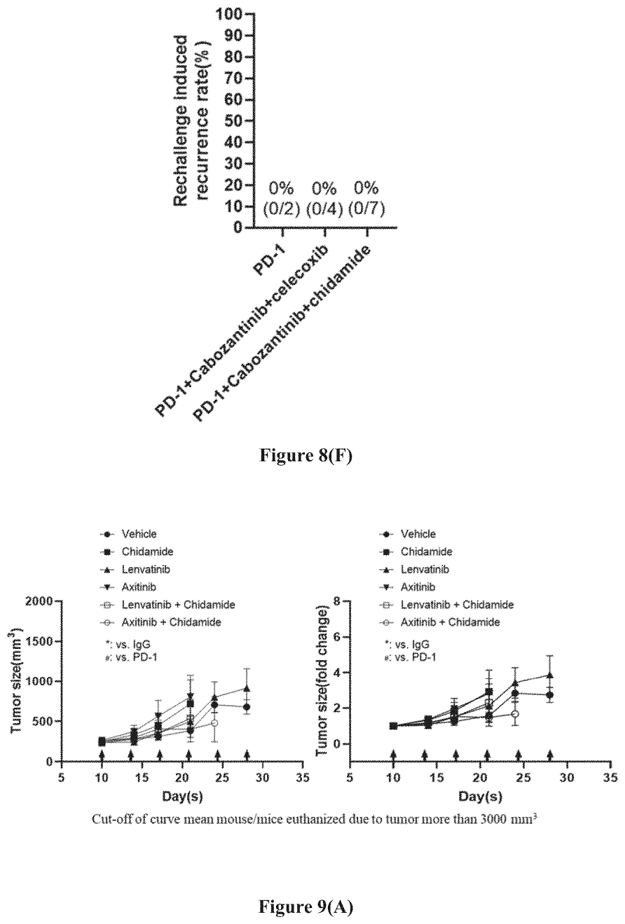

We were very interested to evaluate multiple TKIs combined with anti-PD-1 Ab to boost immune response rate in CT26-bearing mice models. As shown in FIG. 7(A), Cabozantinib, Ibrutinib, Axitinib, and Olaparib, a poly ADP-ribose polymerase inhibitor (PARPi), was combined with anti-PD-1 Ab. The regimen of Cabozantinib combined with anti-PD-1 Ab markedly inhibited tumor growth in comparison with anti-PD-1 Ab combined with Ibrutinib, Axitinib, and Olaparib treatment. The individual tumor sizes (fold change) and ORR as shown in FIG. 7(B) indicated that anti-PD-1 antibody (2.5 mg/kg) group achieved 1 CR, 1 SD and 7 PD, with the ORR (objective response rate) 11%; Cabozantinib (30 mg/kg) combined with anti-PD-1 Ab (2.5 mg/kg) group achieved 1 CR, 7 SD and 1 PD, with the ORR 11%; Axitinib (12.5 mg/kg) combined with anti-PD-1 Ab (2.5 mg/kg) group achieved 1 CR, 2 SD and 6 PD, with the ORR 11%; Ibrutinib (6 mg/kg) combined with anti-PD-1 Ab (2.5 mg/kg) group achieved 1 CR, 1 SD and 7 PD, with the ORR 11%; Olaparib (50 mg/kg) combined with anti-PD-1 Ab (2.5 mg/kg) group achieved 1 SD and 8 PD, with the ORR 0%. In this study, Cabozantinib (30 mg/kg) combined with anti-PD-1 Ab (2.5 mg/kg) achieved the best anti-tumor effect for the control of TME. Next, we were interested to evaluate whether anti-PD-1 Ab combined with Carbozantinib plus COX-2 inhibitor or HDAC inhibitor could boost immune response rate. It was suggested the possibility that addition of a COX-2 inhibitor to inhibit PGE2 synthesis or an HDAC inhibitor to the regimen of Cabozantinib combined with anti-PD-1 Ab might improve the anti-cancer activity. As shown in FIG. 8(A), anti-PD-1 antibody (2.5 mg/kg) combined with Cabozantinib (30 mg/kg) plus Celecoxib (50 mg/kg) or plus Chidamide-k30 (50 mg/kg) regimen achieved very excellent effects in inhibiting tumor growth in comparison with anti-PD-1 Ab treatment alone. Celecoxib is a selective COX-2 inhibitor, and Chidamide is a benzamide-based HDAC inhibitor, selectively inhibiting HDACs 1, 2, 3, and 10. The individual tumor sizes (fold change) and ORR as shown in FIG. 8(B) indicated that anti-PD-1 antibody (2.5 mg/kg) group achieved 1 CR, 1 PR, 2 SD and 4 PD, with the ORR 25%; anti-PD-1 Ab (2.5 mg/kg) combined with Cabozantinib (30 mg/kg) plus Celecoxib (50 mg/kg) group achieved 4 CR and 3 SD, with the ORR 57%; anti-PD-1 Ab (2.5 mg/kg) combined with Cabozantinib (30 mg/kg) plus Chidamide-k30 (50 mg/kg) group achieved 6 CR and 1 PR, with the ORR at 100%. These data suggested that anti-PD-1 Ab (2.5 mg/kg) combined with Cabozantinib (30 mg/kg) plus Chidamide-k30 (50 mg/kg) group achieved better ORR for the control of TME in comparison with anti-PD-1 Ab (2.5 mg/kg) combined with Cabozantinib (30 mg/kg) plus Celecoxib (50 mg/kg) group. This is the first time to discover that TKI plus HDAC inhibitor combined with ICI significantly boosted the immune response rate, which may be attributable to the regulation of TME. There was no significant loss in mice body weight by different regimen treatments as shown in FIG. 8(C). CT26 tumor-bearing mice were euthanized when tumor volume reached 3000 mm³ after implantation. As shown in FIG. 8(D), anti-PD-1 Ab combined with Cabozatinib plus Chidamide-k30 regimen was very powerful in prolonging the survival in comparison with the other groups, achieving 100% survival rate by day 60. This result suggested that Cabozatinib plus Chidamide-k30 may possess potent immune regulation activity in TME. Next, the recurrence rate in each group was evaluated as shown in FIG. 8(E). The result demonstrated that anti-PD-1 Ab, anti-PD-1 Ab combined with Cabozantinib plus Chidamide-k30 or Celecoxib group had no relapses. In rechallenge experiment, as outlined in FIG. 6(F), the immunity activated by treatment with anti-PD-1 Ab, anti-PD-1 Ab combined with Cabozantinib plus Chidamide-k30 or Celecoxib group was studied. As shown in FIG. 8(F), there was no occurrence of tumor growth after rechallenge. The result demonstrated that these PR and CR mice achieved 100% overall immune memory to recognize CT26 cells inoculated during rechallenge. This showed that the regimen of anti-PD-1 Ab combined Cabozantinib plus Chidamide-k30 or Celecoxib was very potent to activate immune memory activity to avoid relapse from occurring.

### Example 4: To Investigate the Anti-Cancer Effect of Tyrosine Kinase Inhibitors (TKIs) Combined with Chidamide-k30 in CT26-Bearing Mice

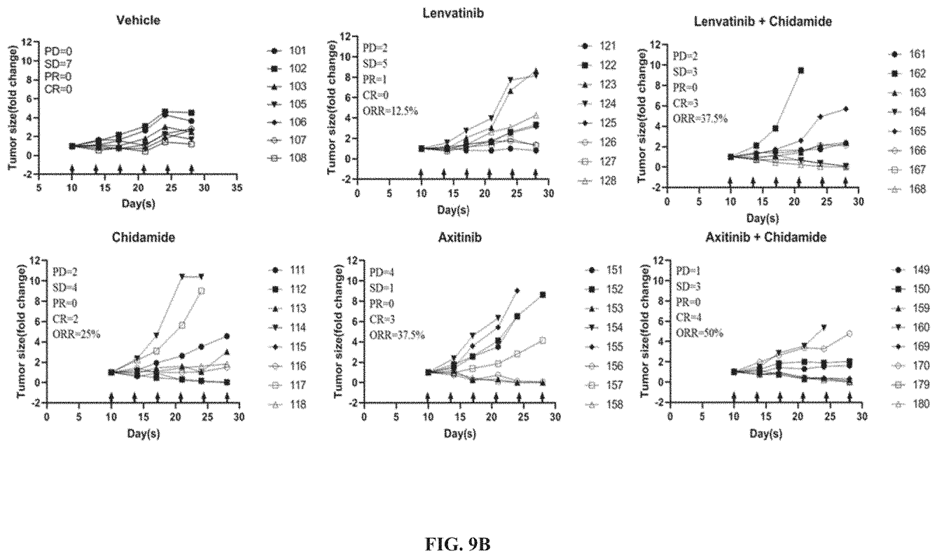

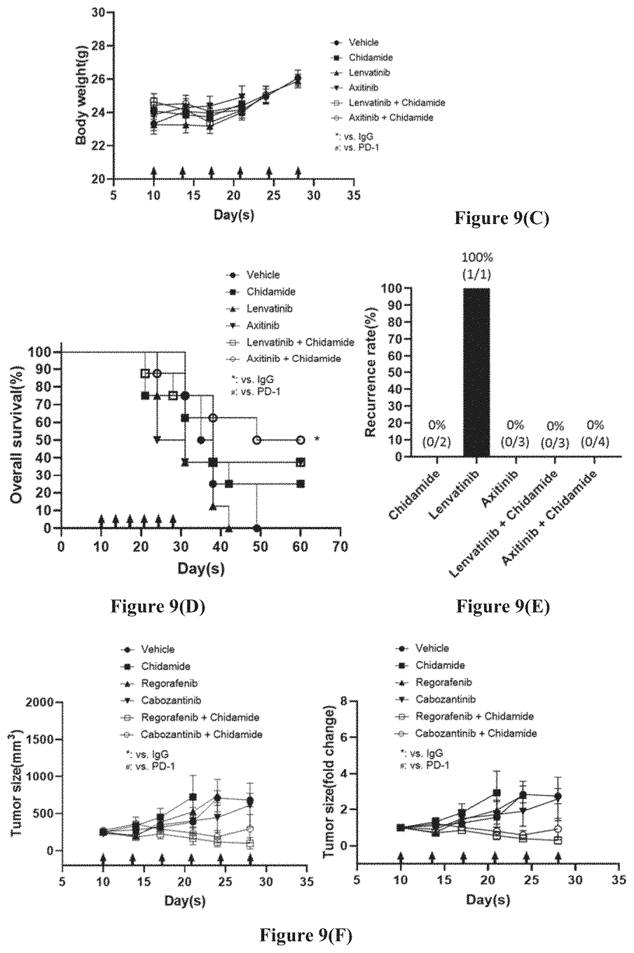

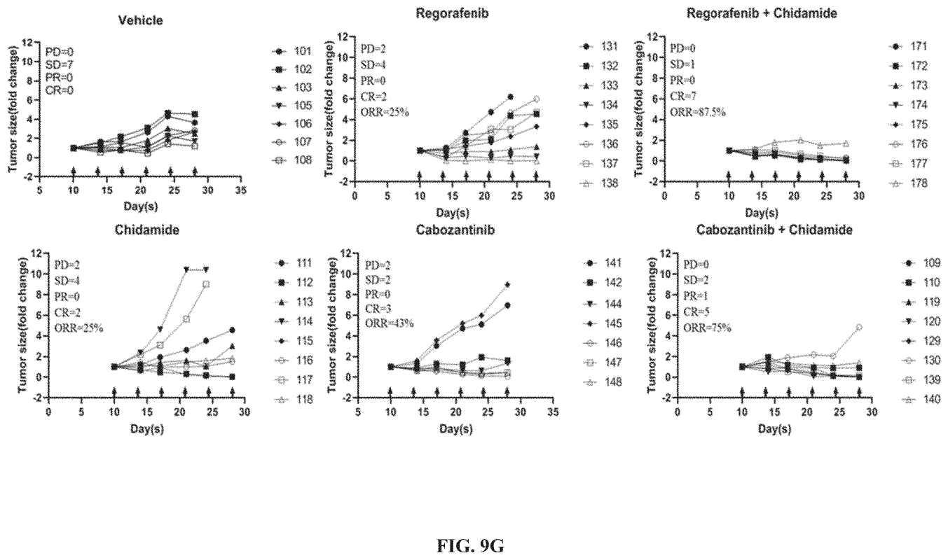

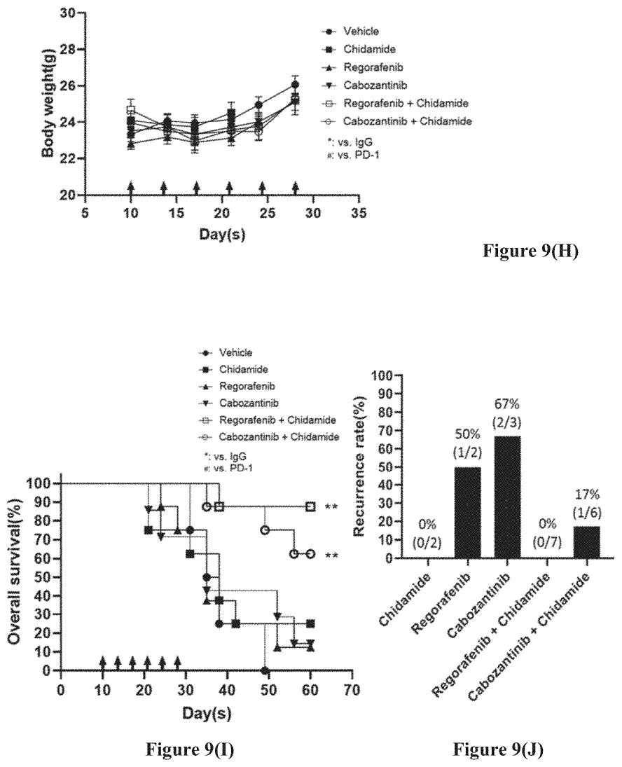

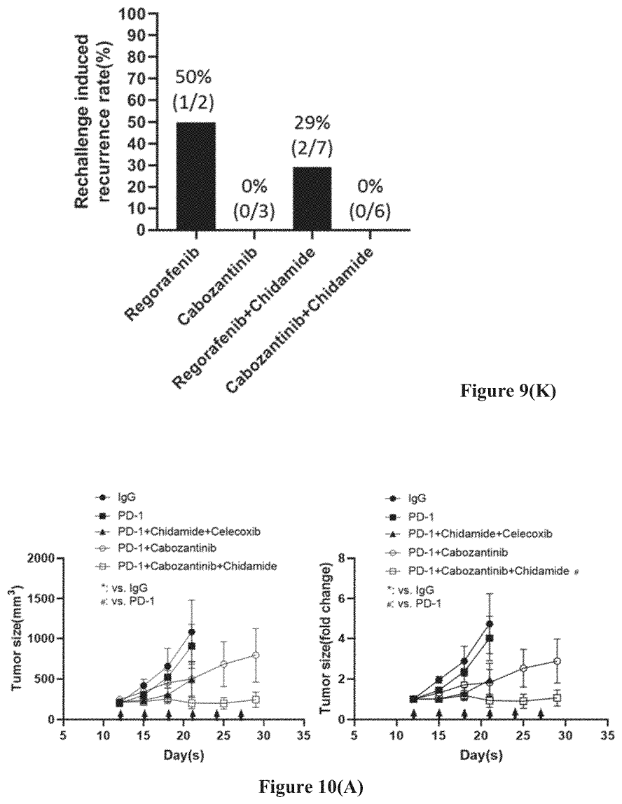

Next, we were interested to study whether TKIs plus Chidamide regimen possessed potent regulation of the TME and significant boosting of the immune response in CT26 tumor-bearing mice models. As shown in FIG. 9(A), several TKIs were combined with Chidamide-k30 to test their capacity for inhibition of tumor growth. The regimen of Chidamide-k30 combined with Lenvatinib or Axitinib was more potent to inhibit tumor growth than Lenvatinib, Axitinib, and Chidamide-k30 treatment alone. The individual tumor sizes (fold change) and ORR as shown in FIG. 9(B) indicated that Chidamide-k30 (50 mg/kg) group achieved 2 CR, 4 SD and 2 PD, with the ORR 25%; Lenvatinib (10 mg/kg) group achieved 1 PR, 5 SD and 2 PD, with the ORR 12.5%; Axitinib (30 mg/kg) group achieved 3 CR, 1 SD, and 4 PD, with the ORR 37.5%; Lenvatinib (10 mg/kg) combined with Chidamide-k30 (50 mg/kg) group achieved 3 CR, 3 SD, and 2 PD, with the ORR 37.5%; Axitinib (30 mg/kg) combined with Chidamide-k30 (50 mg/kg) group achieved 4 CR, 3 SD, and 1 PD, with the ORR 50%. It seems that Chidamide-k30 combined with Lenvatinib or Axitinib possessed an additive effect on the regulation of TME which was demonstrated in the immune response rate in CT26 tumor-bearing mice. There was no significant loss in mice body weight in different treatments as shown in FIG. 9(C). CT26 tumor-bearing mice were euthanized when tumor volume reached 3000 mm³ after implantation. Chidamide-k30 combined with Axitinib or Lenvatinib showed increased survival rate in comparison with Lenvatinib or Chidamide-k30 treatment alone as shown in FIG. 9(D). The recurrence rate was also evaluated as shown in FIG. 9(E). The result suggested that TKIs combined with Chidamide-k30 may possess more potent activity to activate the immune system to avoid a relapse. To further prove the theory, we have studied other TKIs combined with Chidamide-k30 in CT26 tumor-bearing mice models. Regorafenib and Cabozatinib were two potent oral multi-kinase inhibitors tested. Cabozatinib is a potent multi-tyrosine kinase inhibitor for inhibition of c-MET, VEGFR1, VEGFR2, VEGFR3, AXL and RET. Regorafenib is an oral multi-kinase inhibitor for inhibition of VEGFR1, VEGFR2, VEGFR3, TIE-2, RET, KIT, and PDGFR. As shown in FIG. 9(F), Chidamide-k30 combined with Regorafenib or Cabozantinib were very potent to inhibit tumor growth in comparison with Chidamide-k30, Regorafenib, and Cabozantinib treatment alone. The individual tumor sizes (fold change) and ORR as shown in FIG. 9(G) indicated that Chidamide-k30 (50 mg/kg) group achieved 2 CR, 4 SD and 2 PD, with the ORR 25%; Regorafenib (30 mg/kg) group achieved 2 CR, 4 SD and 2 PD, with the ORR 25%; Cabozantinib (30 mg/kg) group achieved 3 CR, 2 SD, and 2 PD, with the ORR 43%; Regorafenib (30 mg/kg) combined with Chidamide-k30 (50 mg/kg) group achieved 7 CR and 1 SD, with the ORR 87.5%; Cabozantinib (30 mg/kg) combined with Chidamide-k30 (50 mg/kg) group achieved 5 CR, 1 PR, and 2 SD, with the ORR 75%. It seems that Chidamide-k30 combined with Regorafenib possessed a more powerful synergistic effect on the regulation of TME and therefore boost the immune response rate in CT26 tumor-bearing mice in comparison with Chidamide-k30 combined with Cabozantinib. Taken together, these data suggested that Chidamide-k30 combined with Regorafenib or Cabozantinib were more potent in regulating TME and significantly augmented the immune response rate in CT26 tumor-bearing mice over the Chidamide-k30 combined with Axitinib or Lenvatinib treatment. As shown in FIG. 9(H), mice body weight did not significantly change. The survival rate of Chidamide-k30 combined with Regorafenib or Cabozantinib groups was evaluated. As shown in FIG. 9(I), Chidamide-k30 combined with Regorafenib was very potent in prolonging the survival over Chidamide-k30 combined with Cabozantinib. The data revealed that Chidamide-k30 combined with Regorafenib or Cabozantinib regimen increased immune response rate and survival rate without being combined with ICIs. This raised the possibility that the regimen of Chidamide-k30 combined with Regorafenib or Cabozantinib has unique TME-regulating properties sufficient to activate CTL or NK cells to kill tumor cells without any combination of ICIs. To further confirm the anti-cancer potency of these two combinations, the recurrence was monitored. As shown in FIG. 9(J), Chidamide-k30 combined with Regorafenib was very powerful in activating the immune system/anti-cancer activity based on the observation that there was no relapse occurred in the 7 mice achieving CR; however in Chidamide-k30 combined with Cabozantinib group and the monotherapy groups, some of the mice achieving CR had a relapse. The rechallenge experiment (outlined as shown in FIG. 6(F)) indicated that Chidamide-k30 combined with Cabozantinib regimen may be more powerful to induce generation of memory T cells than Chidamide-k30 combined with Regorafenib regimen as shown in FIG. 9(K). Based on the results of recurrence and the rechallenge experiments, it seems very possible that the 7 mice achieving CR after treatment with Chidamide-k30 combined with Regorafenib did not have relapse due to complete tumor eradiation, however later on some of the mice (2/7) did not successfully develop or only partially activate immune memory, resulting in the growth of tumor inoculated during rechallenge.

### Example 5: To Investigate the Anti-Cancer Activity of Anti-PD-1 Ab Combined with Carbozantinib or Regorafenib Plus Chidamide-k30 in CT26-Bearing Mice

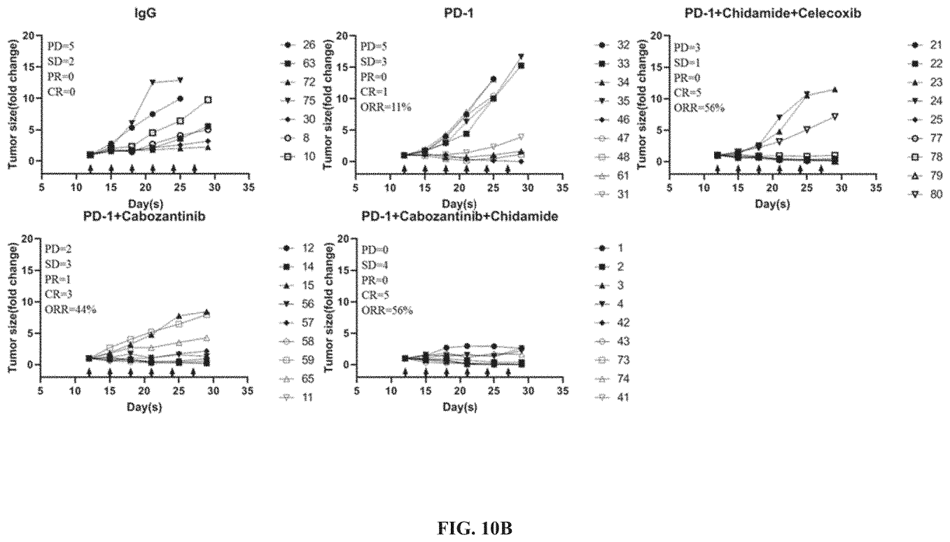

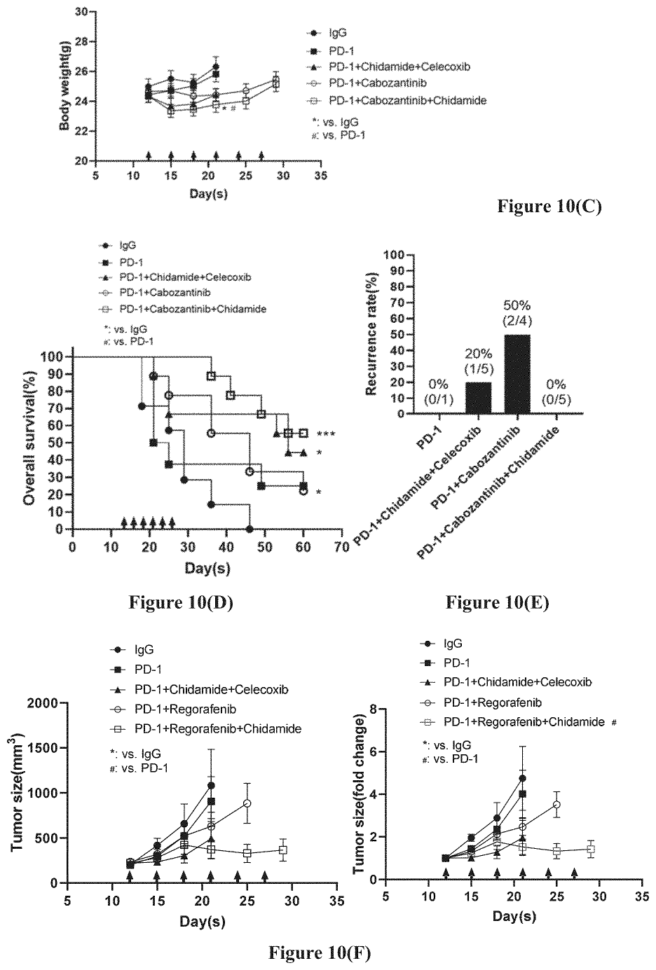

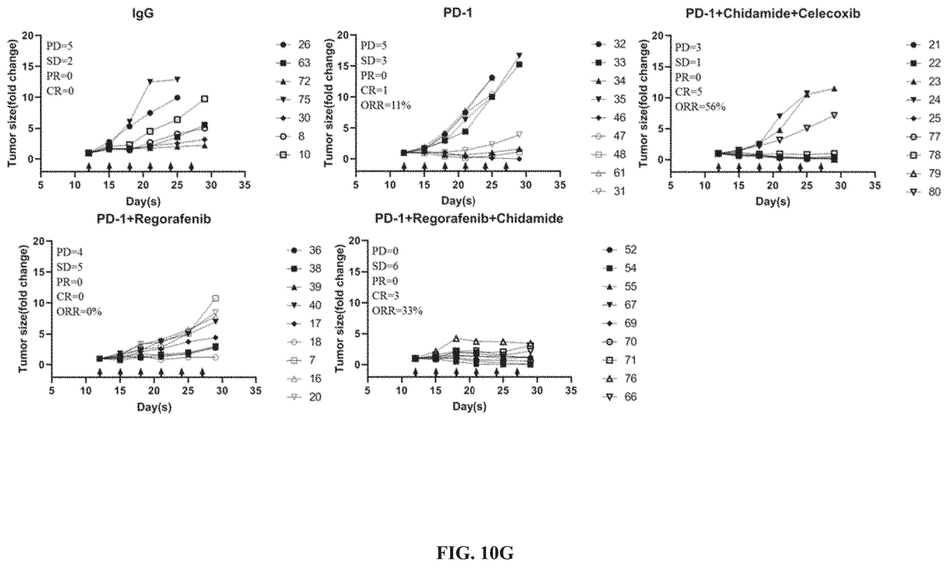

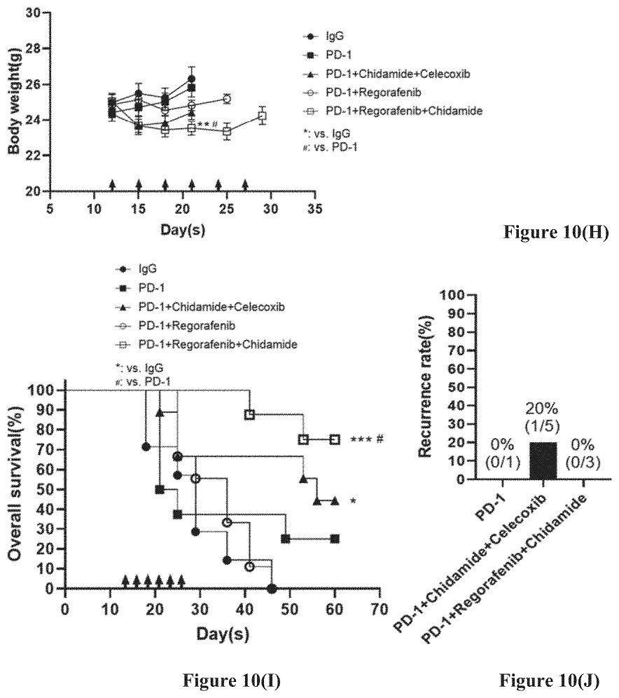

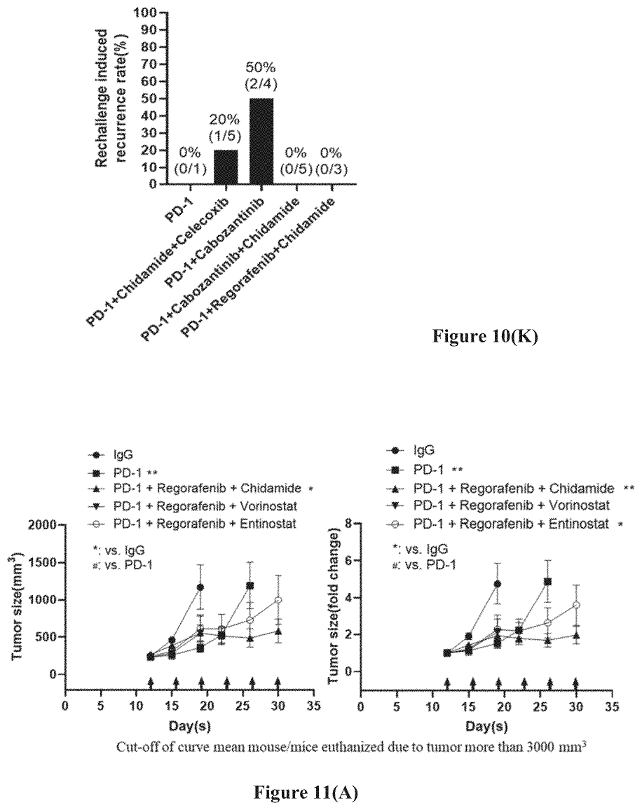

In FIG. 9, our results demonstrated that Chidamide-k30 combined with Cabozantinib or Regorafenib possessed very potent inhibition of tumor growth activity. Next, we were interested to study the addition of anti-PD-1 Ab to the combination with Cabozantinib or Regorafenib plus Chidamide-k30 regimens in CT26 tumor-bearing mice models. As shown in FIG. 10(A), anti-PD-1 Ab combined with Cabozantinib plus Chidamide-k30 regimen was more powerful in inhibiting tumor growth than anti-PD-1 Ab combined with Cabozantinib regimen or anti-PD-1 Ab combined with Chidamide-k30 plus Celecoxib regimen as positive control (It has previously been shown to be a very promising combination of treatment for immunotherapy). This result suggested that Cabozantinib may be a more effective drug than Celecoxib in the triple combination. From the data it was suggested that Cabozantinib may more fully control TME over Celecoxib. The individual tumor sizes (fold change) and ORR as shown in FIG. 10(B) indicated that anti-PD-1 Ab (2.5 mg/kg) group achieved 1 CR, 3 SD and 5 PD, with the ORR 11%; anti-PD-1 Ab (2.5 mg/kg) combined with Chidamide-k30 (50 mg/kg) plus Celecoxib (50 mg/kg) group (as positive control) achieved 5 CR, 1 SD and 3 PD, with the ORR 56%; anti-PD-1 Ab (2.5 mg/kg) combined with Cabozantinib (30 mg/kg) group achieved 3 CR, 1 PR, 3 SD, and 2 PD, with the ORR 44.0%; anti-PD-1 Ab (2.5 mg/kg) combined with Cabozantinib (30 mg/kg) plus Chidamide-k30 (50 mg/kg) group achieved 5 CR and 4 SD, with the ORR 56%. It seems that both groups of anti-PD-1 Ab combined with Cabozantinib plus Chidamide-k30 group and positive control group achieved ORR 56%. However, it seemed that tumors in each mouse treated with anti-PD-1 Ab combined with Cabozantinib plus Chidamide-k30 had more significant inhibition of growth compared to those treated with anti-PD-1 Ab combined with Chidamide-k30 plus Celecoxib (Tumors in three mice were not inhibited as shown in FIG. 10(B)). As shown in FIG. 10(C), treatment with anti-PD-1 Ab combined with Cabozantinib plus Chidamide-k30 initially caused weight drop, but eventually the mice body weight recovered after consecutive treatment. The survival rate of anti-PD-1 Ab combined with Cabozantinib plus Chidamide-k30 group vs. anti-PD-1 Ab combined with Cabozantinib group was evaluated. As shown in FIG. 10(D), anti-PD-1 Ab combined with Cabozantinib plus Chidamide-k30 regimen was more potent in prolonging survival than anti-PD-1 Ab combined with Cabozantinib regimen. The CT26 tumor-bearing mice were euthanized when tumor volume reached 3000 mm³ after implantation. This result also suggested that Chidamide is a very important component to improve the anti-PD-1 Ab combined with Cabozantinib regimen for significant boosting of ORR and survival rate in CT26 tumor-bearing mice. Also based on the evaluation of the recurrence rate as shown in FIG. 10(E), anti-PD-1 Ab combined with Cabozantinib plus Chidamide-k30 regimen was more powerful in avoiding relapse in comparison with anti-PD-1 Ab combined with Cabozantinib regimen. This result also demonstrated that anti-PD-1 Ab combined with Cabozantinib plus Chidamide-k30 group fully activated the immune system to monitor cancer cells and avoid relapse. Next, we were interested to evaluate the triple combination regimen of anti-PD-1 Ab combined with Regorafenib plus Chidamide-k30. As shown in FIG. 10(F), anti-PD-1 Ab (2.5 mg/kg) combined with Regorafenib (30 mg/kg) plus Chidamide-k30 (50 mg/kg) regimen was more powerful in inhibiting tumor growth in comparison with anti-PD-1 Ab (2.5 mg/kg) combined with Regorafenib (30 mg/kg) regimen. Anti-PD-1 Ab (2.5 mg/kg) was combined with Chidamide-k30 (50 mg/kg) plus Celecoxib (50 mg/kg) group as positive control. This result also demonstrated that Chidamide was a key component to improve the regimen of anti-PD-1 Ab combined with Regorafenib to significantly boost the immune response rate for suppression of tumor growth. The individual tumor sizes (fold change) and ORR as shown in FIG. 10(G) indicated that anti-PD-1 Ab (2.5 mg/kg) group achieved 1 CR, 3 SD and 5 PD, with the ORR 11%; anti-PD-1 Ab (2.5 mg/kg) combined with Chidamide-k30 (50 mg/kg) plus Celecoxib (50 mg/kg) group (as positive control) achieved 5 CR, 1 SD and 3 PD, with the ORR 56%; anti-PD-1 Ab (2.5 mg/kg) combined with Regorafenib (30 mg/kg) group achieved 5 SD, and 4 PD, with the ORR 0%; anti-PD-1 Ab (2.5 mg/kg) combined with Regorafenib (30 mg/kg) plus Chidamide-k30 (50 mg/kg) group achieved 3 CR and 6 SD, with the ORR 33%. Although the ORR in anti-PD-1 Ab (2.5 mg/kg) combined with Regorafenib (30 mg/kg) plus Chidamide-k30 (50 mg/kg) group is lower than that of the anti-PD-1 Ab combined with Chidamide-k30 plus Celecoxib positive control group, the tumor growth was significantly inhibited in each mouse as compared to the positive control group in which tumors in three mice were not inhibited as shown in FIG. 10G). As shown in FIG. 10(H), anti-PD-1 Ab combined with Regorafenib plus Chidamide-k30 treatment initially caused weight loss, but eventually the mice body weight recovered after consecutive treatment. The survival rate of anti-PD-1 Ab combined with Regorafenib plus Chidamide-k30 group vs. anti-PD-1 Ab combined with Regorafenib group was evaluated. As shown in FIG. 10(I), anti-PD-1 Ab combined with Regorafenib plus Chidamide-k30 regimen was more powerful in prolonging the survival than anti-PD-1 Ab combined with Regorafenib regimen. This result once more proved that Chidamide is a very important component for contribution to the regimen of anti-PD-1 Ab combined with Regorafenib to significantly boost survival rate in CT26 tumor-bearing mice. We were very surprised that the regimen of anti-PD-1 Ab combined with Regorafenib plus Chidamide-k30 ORR was only 33%, but overall survival rate was as high as 77%. Obviously, the regimen has very strong modulation of tumor immunologic activity, and although the drug was stopped being given, it continued to shrink the tumor. Similar results can be found in mice with drug resistance to first line therapy with anti-PD-1 Ab, which were then treated with second line therapy of anti-PD-1 Ab combined with Regorafenib/Cabozantinib plus Chidamide-k30 as shown in FIGS. 4 & 5(D). The recurrence rate was evaluated as shown in FIG. 10(J). In the Anti-PD-1 Ab combined with Regorafenib plus Chidamide-k30 group, none of the mice had a recurrence. The rechallenge experiment was performed and the results are shown in FIG. 10(K). Both anti-PD-1 Ab combined with Regorafenib plus Chidamide-k30 group and anti-PD-1 Ab combined with Cabozantinib plus Chidamide-k30 group had very powerful immune activation to prevent the growth of the tumor inoculated by rechallenge process. Furthermore, we also found that overall immunity rate was lower in mice with CR or PR after treated with anti-PD-1 Ab combined with Cabozantinib. Based on the results of activation of immunity, it was suggested that the regimens of anti-PD-1 Ab combined with multi-kinase inhibitors such as Regorafenib or Cabozantinib plus Chidamide-k30 activated a specific immune memory and therefore performed strong anti-cancer activity. It is beneficial to control TME to improve tumor immune response rate and avoid recurrence. Chidamide, a subtype-selective HDACs 1, 2, 3, and 10 inhibitors and a potent epigenetic immunomodulator, has been approved for R/R PTCL (relapsed/refractory Peripheral T-Cell Lymphoma) and ER⁺/Her-2⁻ breast cancer treatment by NMPA of China.

### Example 6: To Investigate the Anti-Cancer Activity of ICIs Combined with Tyrosine Kinase Inhibitors (TKIs) Plus Histone Deacetylase Inhibitors (HDACis) in CT26-Bearing Mice

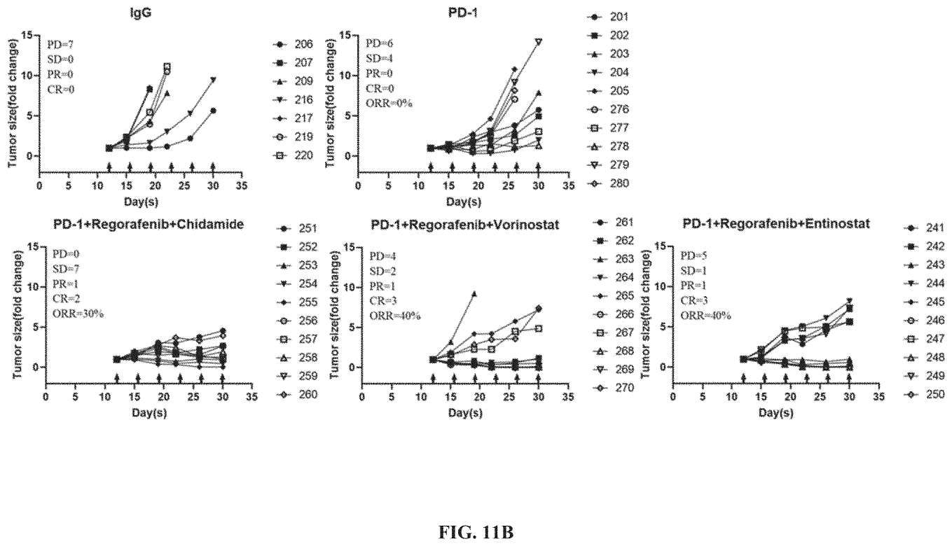

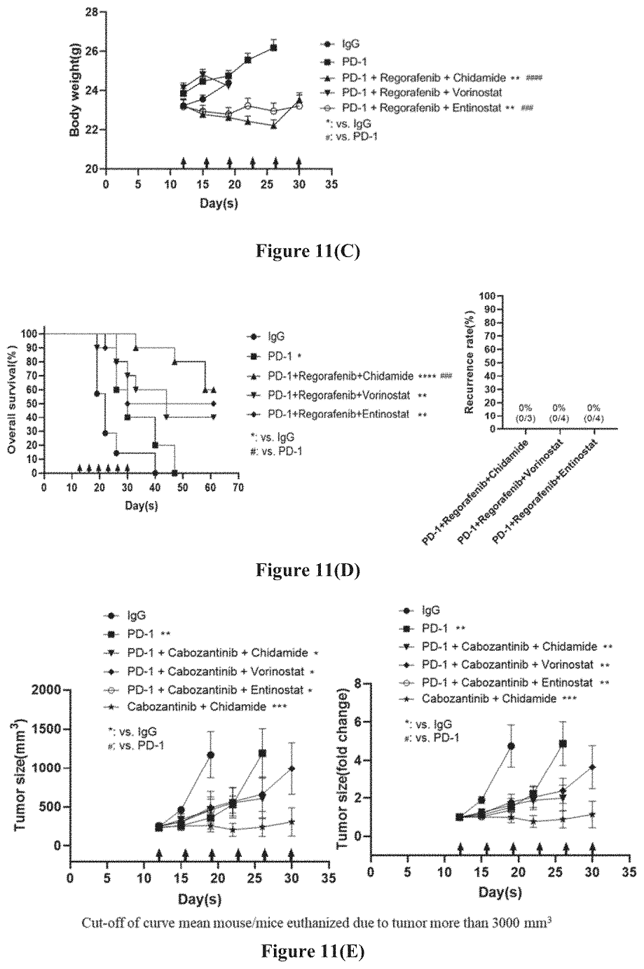

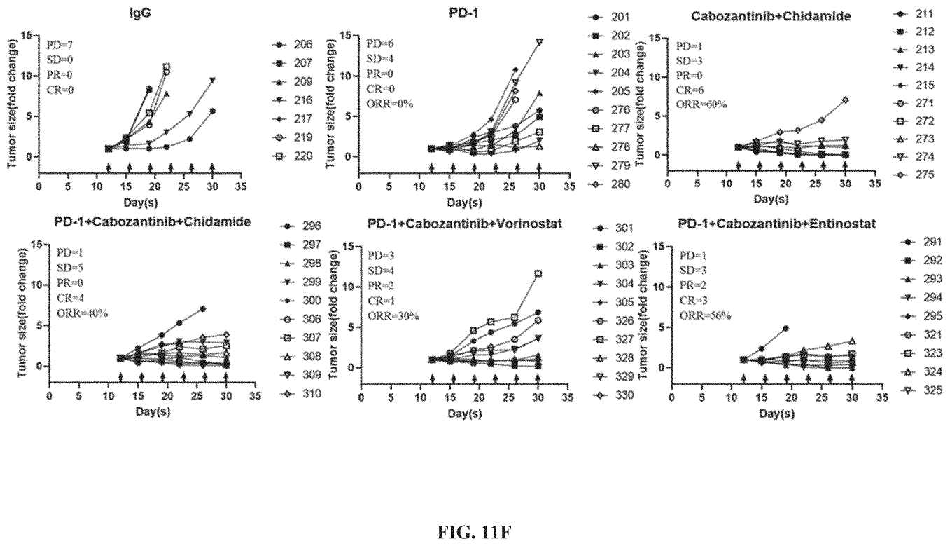

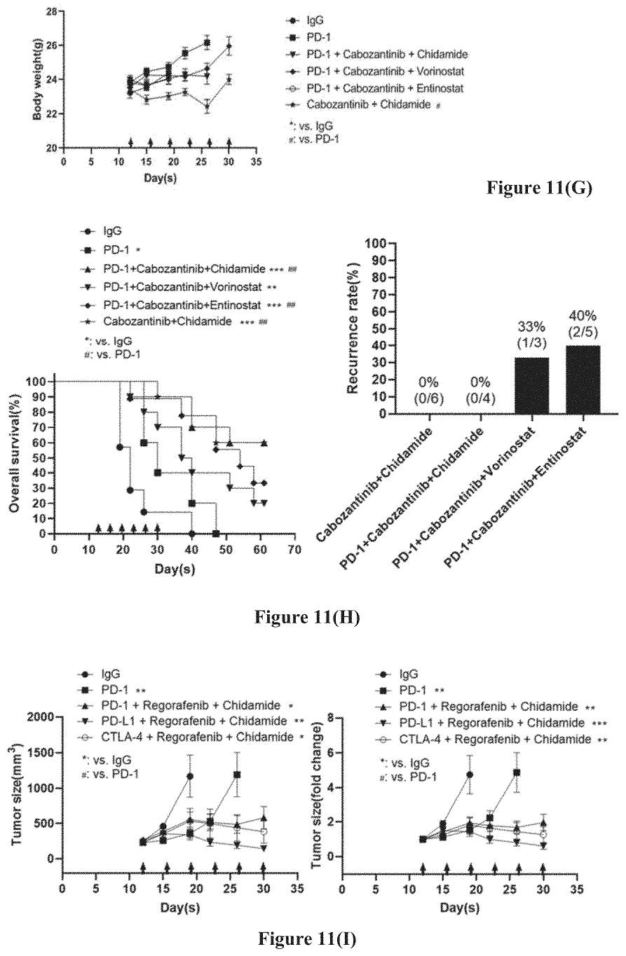

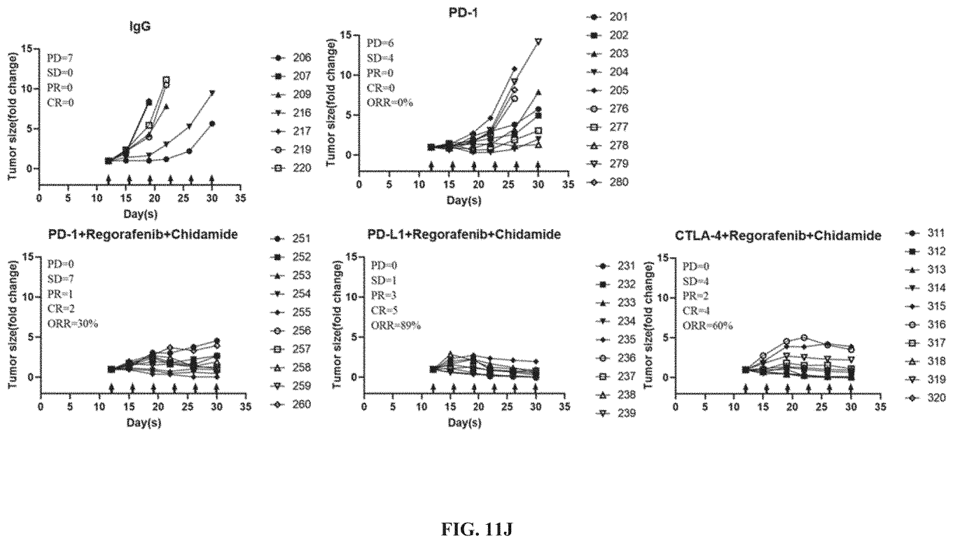

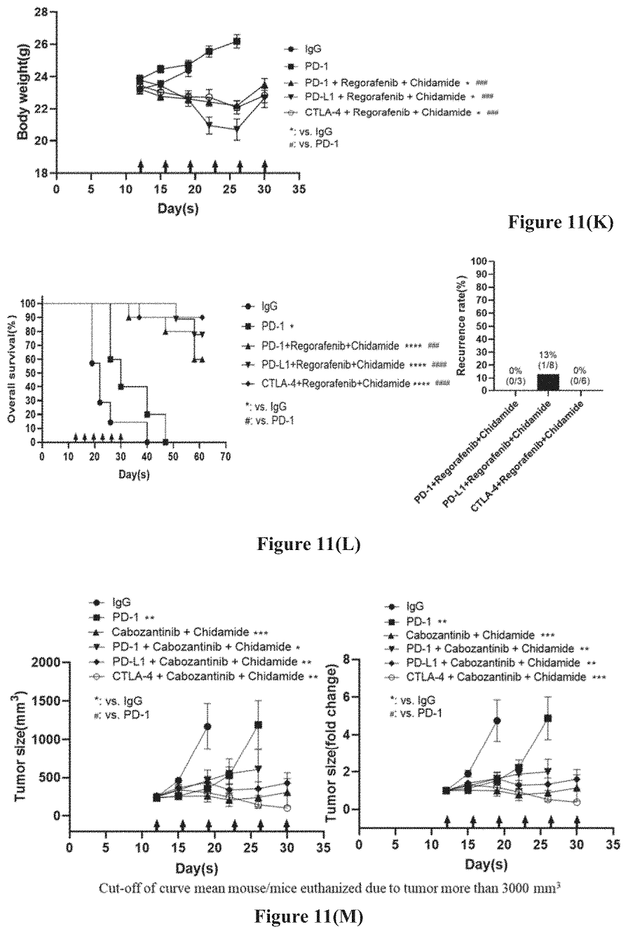

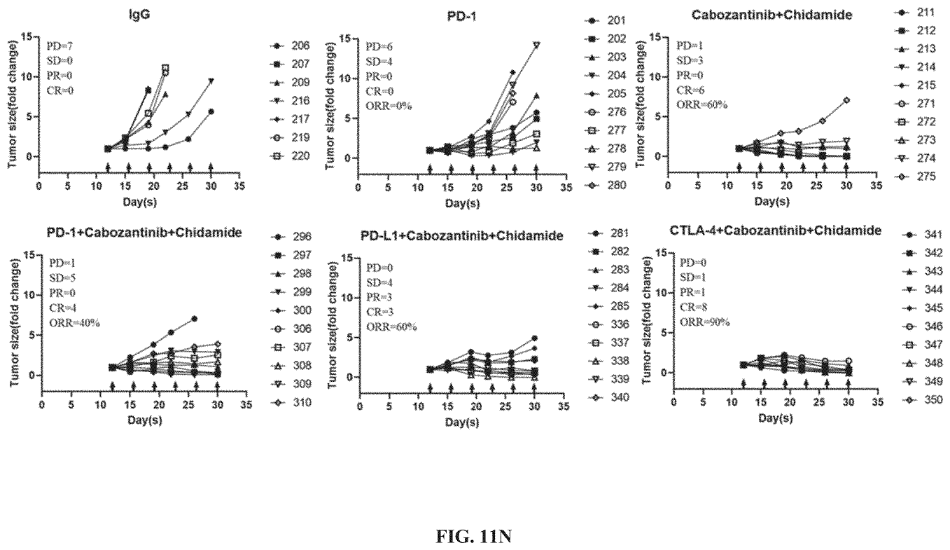

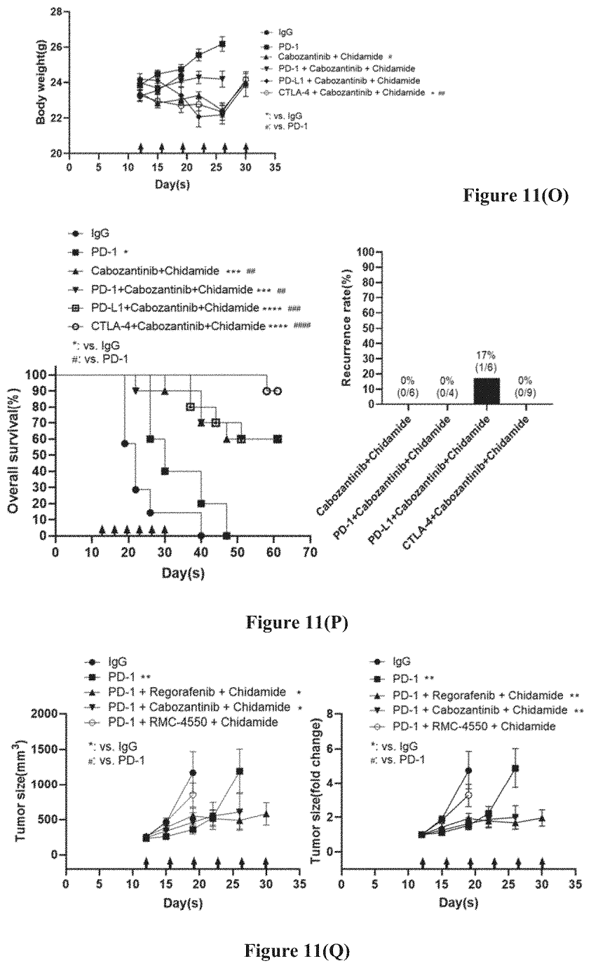

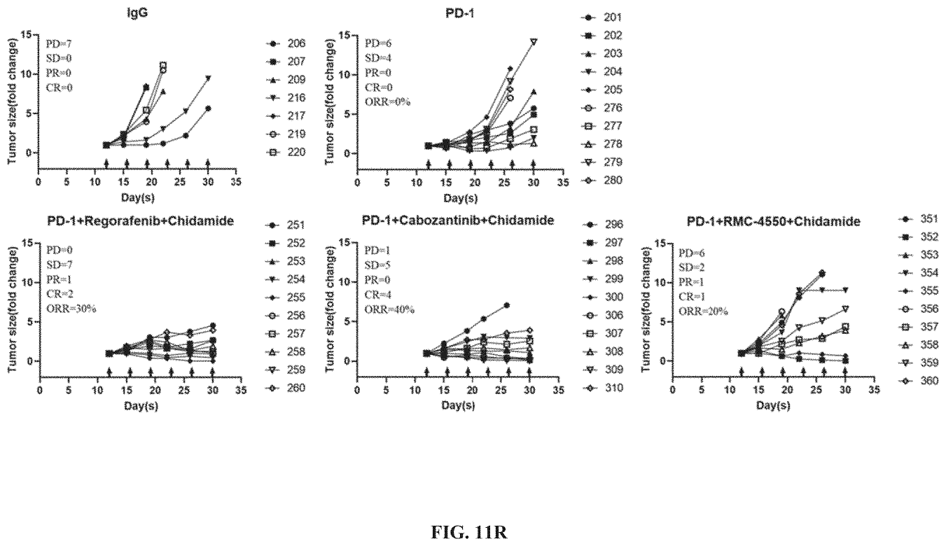

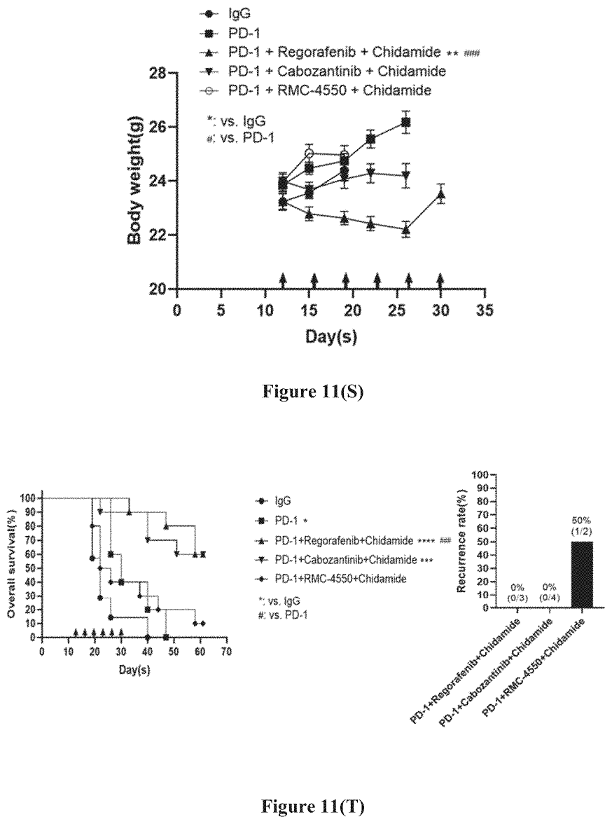

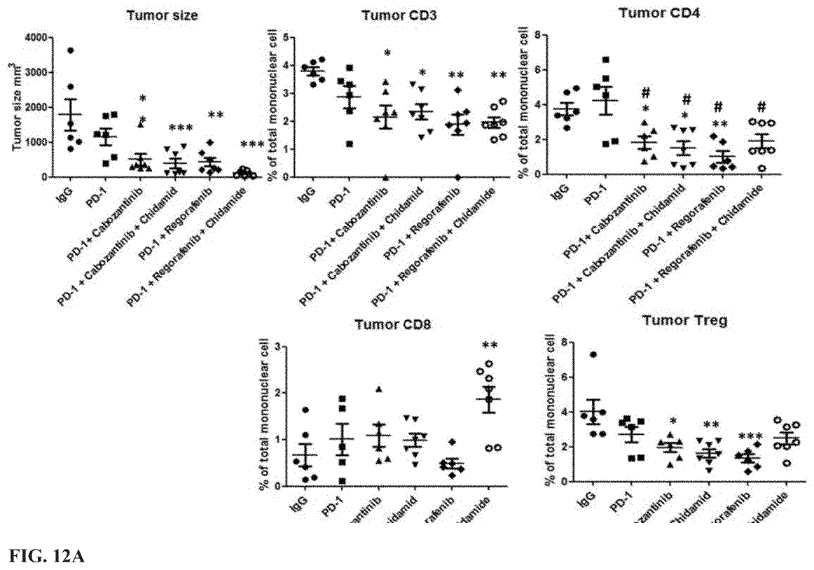

The potency and the anticancer mechanisms of anti-PD-1 Ab combined with TKIs plus HDACis were further studied in CT26 tumor-bearing mice. As shown in FIG. 11(A), we evaluated the anti-PD-1 Ab combined with Regorafenib plus different HDAC inhibitors such as Chidamide (inhibition of HDACs 1, 2, 3, and 10), Vorinostat (SAHA, a pan-HDAC inhibitor), and Entinostat (inhibition of HDACs 1, 2, and 3). The result demonstrated that anti-PD-1 Ab (2.5 mg/kg) combined with Regorafenib (30 mg/kg) plus Chidamide-k30 (50 mg/kg) possessed more potent inhibition of tumor growth than anti-PD-1 Ab combined with Vorinostat or Entinostat. The individual tumor sizes (fold change) and ORR as shown in FIG. 11(B) indicated that anti-PD-1 Ab (2.5 mg/kg) group achieved 4 SD and 6 PD, with the ORR (objective response rate) 0%; anti-PD-1 Ab (2.5 mg/kg) combined with Regorafenib (30 mg/kg) plus Chidamide-k30 (50 mg/kg) group achieved 2 CR, 1 PR and 7 SD, with the ORR 30%; anti-PD-1 Ab (2.5 mg/kg) combined with Regorafenib (30 mg/kg) plus Vorinostat (150 mg/kg) group achieved 3 CR, 1 PR, 2 SD, and 4 PD, with the ORR 40%; anti-PD-1 Ab (2.5 mg/kg) combined with Regorafenib (30 mg/kg) plus Entinostat (20 mg/kg) group achieved 3 CR, 1 PR, 1 SD and 5 PD, with the ORR 40%. Although the ORR in anti-PD-1 Ab (2.5 mg/kg) combined with Regorafenib (30 mg/kg) plus Chidamide-k30 (50 mg/kg) group is lower (ORR 30%) than that of the anti-PD-1 Ab combined with Regorafenib plus Vorinostat (ORR 40%) or Entinostat (ORR 40%) groups, tumor growth was significantly inhibited in each mouse (none of the mice got the PD as shown in FIG. 11(B)) in anti-PD-1 Ab (2.5 mg/kg) combined with Regorafenib (30 mg/kg) plus Chidamide-k30 (50 mg/kg) group compared to anti-PD-1 Ab combined with Regorafenib plus Vorinostat or Entinostat group (4 PD and 5 PD, respectively). As shown in FIG. 11(C), anti-PD-1 Ab combined with Regorafenib plus Chidamide-k30 or Entinostat groups initially dropped weight, but then the mice body weight eventually recovered. As shown in FIG. 11(D), the overall survival rate is shown below: anti-PD-1 Ab combined with Regorafenib plus Chidamide-k30 group >anti-PD-1 Ab combined with Regorafenib plus Entinostat group >anti-PD-1 Ab combined with Regorafenib plus Vorinostat group >anti-PD-1 Ab group. Notably, none of the mice that obtained ORR were found to have recurrence (right panel of FIG. 11(D)). Next, the triple combinations with Cabozantinib were tested. As shown in FIG. 11(E), Cabozantinib (30 mg/kg) combined with Chidamide-k30 (50 mg/kg) group as a control (also studied in FIG. 9) showed more potent tumor growth inhibition than anti-PD-1 Ab (2.5 mg/kg) combined with Cabozantinib (30 mg/kg) plus Chidamide-k30 (50 mg/kg) group. However, anti-PD-1 Ab (2.5 mg/kg) combined with Cabozantinib (30 mg/kg) plus Chidamide-k30 (50 mg/kg) or Entinostat (20 mg/kg) regimens were more potent in inhibiting tumor growth than anti-PD-1 Ab (2.5 mg/kg) combined with Cabozantinib (30 mg/kg) plus Vorinostat (150 mg/kg) regimen or anti-PD-1 Ab (2.5 mg/kg) treatment alone. The individual tumor sizes (fold change) and ORR as shown in FIG. 11(F) indicated that anti-PD-1 Ab (2.5 mg/kg) group achieved 4 SD and 6 PD, with the ORR (objective response rate) 0%; anti-PD-1 Ab (2.5 mg/kg) combined with Cabozantinib (30 mg/kg) plus Chidamide-k30 (50 mg/kg) group achieved 4 CR, 5 SD, and 1 PD with the ORR 40%; anti-PD-1 Ab (2.5 mg/kg) combined with Cabozantinib (30 mg/kg) plus Vorinostat (150 mg/kg) group achieved 1 CR, 2 PR, 4 SD, and 3 PD, with the ORR 30%; anti-PD-1 Ab (2.5 mg/kg) combined with Cabozantinib (30 mg/kg) plus Entinostat (20 mg/kg) group achieved 3 CR, 2 PR, 3 SD and 1 PD, with the ORR 56%. The Cabozantinib (30 mg/kg) combined with Chidamide-k30 (50 mg/kg) group achieved 6 CR, 3 SD and 1 PD, with the ORR 60%, which showed a similar result as compared to FIG. 9(G). Although the ORR in anti-PD-1 Ab (2.5 mg/kg) combined with Cabozantinib (30 mg/kg) plus Chidamide-k30 (50 mg/kg) group is lower (ORR 40%) than that of the anti-PD-1 Ab combined with Cabozantinib plus Entinostat (ORR 56%) group, the tumor growth was significantly inhibited in each mouse (only one mouse got PD as shown in FIG. 11(F) in anti-PD-1 Ab (2.5 mg/kg) combined with Cabozantinib (30 mg/kg) plus Chidamide-k30 (50 mg/kg) or Entinostat (20 mg/kg) groups compared to anti-PD-1 Ab combined with Cabozantinib plus Vorinostat or anti-PD-1 Ab treatment alone (3 PD and 6 PD, respectively). As shown in FIG. 11(G), only Cabozantinib combined with Chidamide-k30 group initially dropped weight, but the mice body weight eventually recovered. As shown in FIG. 11(H), the overall survival rate is shown below: anti-PD-1 Ab combined with Cabozantinib plus Chidamide-k30 group >anti-PD-1 Ab combined with Cabozantinib plus Entinostat group >anti-PD-1 Ab combined with Cabozantinib plus Vorinostat group >anti-PD-1 Ab group. Although with a higher ORR, the Cabozantinib (30 mg/kg) combined with Chidamide-k30 (50 mg/kg) group had a same survival rate as compared to anti-PD-1 Ab combined with Cabozantinib plus Chidamide-k30 group. This result again demonstrated that anti-PD-1 Ab combined with Cabozantinib plus Chidamide-k30 possessed very potent immune regulation activity, although the drug had stopped being given. There was no recurrence in anti-PD-1 Ab combined with Cabozantinib plus Chidamide-k30 group and Cabozantinib (30 mg/kg) combined with Chidamide-k30 (50 mg/kg) group. Next, we were interested to evaluate the activities of tumor growth inhibition in combinations with different ICIs, such as anti-PD-1/anti-PD-L1/anti-CTLA-4 Ab combined with Regorafenib/Cabozantinib plus Chidamide-k30 regimens in CT26 tumor-bearing mice. As shown in FIG. 11(I), anti-PD-L1 Ab (2.5 mg/kg) combined with Regorafenib (30 mg/kg) plus Chidamide-k30 (50 mg/kg) regimen was more powerful in inhibiting tumor growth than anti-CTLA-4 Ab (2.5 mg/kg) or anti-PD-1 Ab (2.5 mg/kg) combined with Regorafenib (30 mg/kg) plus Chidamide-k30 (50 mg/kg) regimes. The individual tumor sizes (fold change) and ORR as shown in FIG. 11(J) indicated that anti-PD-1 Ab (2.5 mg/kg) group achieved 4 SD and 6 PD, with the ORR 0%; anti-PD-1 Ab (2.5 mg/kg) combined with Regorafenib (30 mg/kg) plus Chidamide-k30 (50 mg/kg) group achieved 2 CR, 1 PR, and 7 SD with the ORR 30%; anti-PD-L1 Ab (2.5 mg/kg) combined with Regorafenib (30 mg/kg) plus Chidamide-k30 (50 mg/kg) group achieved 5 CR, 3 PR, and 1 SD, with the ORR 89%; anti-CTLA-4 Ab (2.5 mg/kg) combined with Regorafenib (30 mg/kg) plus Chidamide-k30 (50 mg/kg) group achieved 4 CR, 2 PR, and 4 SD, with the ORR 60%. The activity that inhibits tumor growth is as follows: anti-PD-L1 Ab combined with Regorafenib plus Chidamide-k30>anti-CTLA-4 Ab combined with Regorafenib plus Chidamide-k30>regimen>anti-PD-1 Ab combined with Regorafenib plus Chidamide-k30 regimen>anti-PD-1 Ab regimen. As shown in FIG. 11(K), only anti-PD-L1 Ab combined with Regorafenib plus Chidamide-k30 group initially severely dropped weight, but then the mice body weight gradually recovered. Anti-CTLA-4/anti-PD-1 Ab combined with Regorafenib plus Chidamide-k30 groups initially had a slight loss in body weight and eventually the mice body weight also recovered. These results demonstrated that anti-PD-L1 Ab combined with Regorafenib plus Chidamide-k30 regimen possessed more potent activity of tumor growth inhibition, but may have had a stronger presence of toxicity and may need further evaluation over other treatment regimens. As shown in FIG. 11(L), we confirmed whether the different ICIs combination with Regorafenib plus Chidamide-k30 groups possessed different overall survival rates. The overall survival rate is shown below: anti-CTLA-4 Ab combined with Regorafenib plus Chidamide-k30 group >anti-PD-L1 Ab combined with Regorafenib plus Chidamide-k30 group >anti-PD-1 Ab combined with Regorafenib plus Chidamide-k30 group >anti-PD-1 Ab group. That the ORR of the two drug combinations (anti-CTLA-4 Ab combined with Regorafenib plus Chidamide-k30 and anti-PD-1 Ab combined with Regorafenib plus Chidamide-k30) was lower than the overall survival rate is likely related to the anti-cancer mechanism of such regimens in the effective regulation of TME. However, only one mouse had recurrence in anti-PD-L1 Ab combined with Regorafenib plus Chidamide-k30 group. This also demonstrated that these regimens were very powerful to activate the immune system to avoid relapse. As shown in FIG. 11(M), anti-CTLA-4 Ab (2.5 mg/kg) combined with Cabozantinib (30 mg/kg) plus Chidamide-k30 (50 mg/kg) was more potent to inhibit tumor growth than the other regimens treatment. The individual tumor sizes (fold change) and ORR as shown in FIG. 11(N) indicated that anti-PD-1 Ab (2.5 mg/kg) group achieved 4 SD and 6 PD, with the ORR 0%; Cabozantinib (30 mg/kg) combined with Chidamide-k30 (50 mg/kg) group achieved 6 CR, 3 SD, and 1 PD with the ORR 60%; anti-PD-1 Ab (2.5 mg/kg) combined with Cabozantinib (30 mg/kg) plus Chidamide-k30 (50 mg/kg) group achieved 4 CR, 5 SD, and 1 PD with the ORR 40%; anti-PD-L1 Ab (2.5 mg/kg) combined with Cabozantinib (30 mg/kg) plus Chidamide-k30 (50 mg/kg) group achieved 3 CR, 3 PR, and 4 SD, with the ORR 60%; anti-CTLA-4 Ab (2.5 mg/kg) combined with Cabozantinib (30 mg/kg) plus Chidamide-k30 (50 mg/kg) group achieved 8 CR, 1 PR, and 1 SD, with the ORR 90%. The activity that inhibits tumor growth is as follows: anti-CTLA-4 Ab combined with Cabozantinib plus Chidamide-k30 regimen>Cabozantinib combined with Chidamide-k30 regimen>anti-PD-L1 Ab combined with Cabozantinib plus Chidamide-k30 regimen>anti-PD-1 Ab combined with Cabozantinib plus Chidamide-k30 regimen. As shown in FIG. 11(O), only anti-PD-1 Ab combined with Cabozantinib plus Chidamide-k30 group generally maintained body weight. However, anti-PD-L1/anti-CTLA-4 Ab combined with Cabozantinib plus Chidamide-k30 or Cabozantinib combined with Chidamide-k30 groups initially severely dropped weight, but then eventually the mice body weight recovered. As shown in FIG. 11(P), the overall survival rate is shown below: anti-CTLA-4 Ab combined with Cabozantinib plus Chidamide-k30 group >anti-PD-1 Ab combined with Cabozantinib plus Chidamide-k30 group=anti-PD-L1 Ab combined with Cabozantinib plus Chidamide-k30 group=Cabozantinib plus Chidamide-k30 group >anti-PD-1 Ab group. Only one mouse had recurrence in anti-PD-L1 Ab combined with Cabozantinib plus Chidamide group. This result also demonstrated that these regimens were very potent to activate the immune system to avoid relapse. Next, we were interested to compare the regimens of anti-PD-1 Ab combined with different TKIs plus Chidamide-k30. As shown in FIG. 11(Q), anti-PD-1 Ab combined with Regorafenib or Cabozantinib plus Chidamide-k30 regimens were more potent in inhibiting tumor growth than other regimen. RMC-4550 was a well-known potent SHP-2 inhibitor and reports had suggested it is powerful in improving ORR when combined with anti-PD-1 Ab in animal models. However, our data showed that RMC-4550 did not have the ability to augment the anti-cancer activity when combined with anti-PD-1 Ab plus Chidamide-k30 regimen over Regorafenib or Cabozantinib in CT26 tumor-bearing mice. The individual tumor sizes (fold change) and ORR as shown in FIG. 11(R) indicated that anti-PD-1 Ab (2.5 mg/kg) group achieved 4 SD and 6 PD, with the ORR 0%; anti-PD-1 Ab (2.5 mg/kg) combined with Regorafenib (30 mg/kg) plus Chidamide-k30 (50 mg/kg) group achieved 2 CR, 1 PR, and 7 SD with the ORR 30%; anti-PD-1 Ab (2.5 mg/kg) combined with Cabozantinib (30 mg/kg) plus Chidamide-k30 (50 mg/kg) group achieved 4 CR, 5 SD, and 1 PD with the ORR 40%; anti-PD-1 Ab (2.5 mg/kg) combined with RMC-4550 (30 mg/kg) plus Chidamide-k30 (50 mg/kg) group achieved 1 CR, 1 PR, 2 SD, and 6 PD with the ORR 20%. The activity that inhibits tumor growth is as follows: anti-PD-1 Ab combined with Cabozantinib plus Chidamide-k30 regimen>anti-PD-1 Ab combined with Regorafenib plus Chidamide-k30 regimen>anti-PD-1 Ab combined with RMC-4550 plus Chidamide-k30 regimen. The mice body weight is shown in FIG. 11(S). As shown in FIG. 11(T), the overall survival rate is shown below: anti-PD-1 Ab combined with Regorafenib plus Chidamide-k30 group=anti-PD-1 Ab combined with Cabozantinib plus Chidamide-k30 group >anti-PD-1 Ab combined with RMC-4550 plus Chidamide-k30 group >anti-PD-1 Ab group. Only anti-PD-1 Ab combined with RMC-4550 plus Chidamide-k30 group had recurrence. As shown in Table 3, the normal ORR assessments are performed 3 days after the last drug administration. However as shown in Table 4, it was surprising to find that after stopping drug administration the tumor in CT26 tumor-bearing mice continued to shrink, so a second ORR assessment was performed 10 days after the last drug was given. Anti-PD-1 Ab (2.5 mg/kg) combined with Regorafenib (30 mg/kg) plus Chidamide-k30 (50 mg/kg) group significantly boosted ORR from 30% to 60% in the second ORR assessment. The 3 mice that originally achieved SD had continued to have tumor shrinkage and achieved 1 PR, and 2 CR, because the drug effects continued to activate CTL and NK in the mice's immune system, resulting in the result of 4 CR, 2 PR, 3 SD and 1 PD in the second assessment. A similar phenomenon was also present in anti-PD-1 Ab (2.5 mg/kg) combined with Regorafenib (30 mg/kg) plus Entinostat (20 mg/kg) group, the ORR increased from 40% to 50%. Furthermore, we found that in the anti-CTLA-4 Ab (2.5 mg/kg) combined with Regorafenib (30 mg/kg) plus Chidamide-k30 (50 mg/kg) group that ORR increased from 60% to 80% and achieved more mice getting CR. Although the ORR of anti-PD-L1 Ab (2.5 mg/kg) combined with Regorafenib (30 mg/kg) plus Chidamide-k30 (50 mg/kg) group did not change, more mice became CR (from 5 to 8). In the anti-PD-1 Ab (2.5 mg/kg) combined with Cabozantinib (30 mg/kg) plus Chidamide-k30 (50 mg/kg) group, the ORR significantly boosted from 40% to 60%. Although the ORR of anti-PD-1 Ab (2.5 mg/kg) combined with Cabozantinib (30 mg/kg) plus Entinostat (20 mg/kg) group did not change, more mice became CR (from 3 to 5). A similar phenomenon was also present in anti-PD-L1/anti-CTLA-4 Ab (2.5 mg/kg) combined with Cabozantinib (30 mg/kg) plus Chidamide-k30 (50 mg/kg) groups that the CR increased from 3 to 5 and 8 to 9, respectively. These data demonstrated that anti-PD-1/anti-PD-L1/anti-CTLA-4 Ab combined with Regorafenib or Cabozantinib plus Chidamide-k30 or Entinostat possessed very potent activity for activation of CTL or NK. This is an important finding that the inherent anti-cancer potency of ICIs combined with TKIs plus HDACis in CT26 tumor-bearing mice was revealed by observation of improved anti-cancer efficacy in the mice with initial SD or PR as an implication of delayed development of enhanced immunity. Finally, the immunity was further studied by rechallenge experiment (as shown in far-right column of Table 4). The study showed that almost all regimens were significantly active in the stimulation of immunity and therefore effectively inhibited proliferation of CT26 cancer cells inoculated by rechallenge. However, only two regimens (anti-PD-1 Ab combined with Cabozantinib plus Entinostat and anti-PD-1 Ab combined with RMC-4550 plus Chidamide) had occurrence of tumor growth.

TABLE 3
The efficacy of HDAC inhibitors plus tyrosine kinase inhibitors
with or without ICI in CT26 tumor-bearing mice model.

| | Initial | | | | | | | | |
|---|---|---|---|---|---|---|---|---|---|
| | Tumor | | | | | | Survival | | |
| | Volume | ORR | | | | | Rate | Relapse* | Immunity^{#} |
| Regimens | (mm³) | (%) | PD | SD | PR | CR | (%) | (recurrence) | (rechallenge) |
| FIG. 6 | | | | | | | | | |
| anti-PD-1 Ab | 190 | 18% | 5 | 4 | 1 | 1 | 18% | 100% | 100% |
| | | | | | | | (2/11) | (2/2) | (2/2) |
| Lenvatinib | | 30% | 2 | 5 | 2 | 1 | 10% | 67% | 67% |
| | | | | | | | (1/10) | (2/3) | (2/3) |
| anti-PD-1 Ab + | | 17% | 3 | 7 | 0 | 2 | 25% | 0% | 100% |
| Lenvatinib | | | | | | | (3/12) | (0/2) | (2/2) |
| FIG. 7 | | | | | | | | | |
| anti-PD-1 Ab | 299 | 11% | 7 | 1 | 0 | 1 | | | |
| anti-PD-1 Ab + | | 11% | 1 | 7 | 0 | 1 | | | |
| Cabozantinib | | | | | | | | | |
| anti-PD-1 Ab + | | 11% | 6 | 2 | 0 | 1 | | | |
| Axitinib | | | | | | | | | |
| anti-PD-1 Ab + | | 11% | 7 | 1 | 0 | 1 | | | |
| Ibrutinib | | | | | | | | | |
| anti-PD-1 Ab + | | 0% | 8 | 1 | 0 | 0 | | | |
| Olaparib | | | | | | | | | |
| FIG. 8 | | | | | | | | | |
| anti-PD-1 Ab | 177 | 25% | 4 | 2 | 1 | 1 | 38% | 0% | 100% |
| | | | | | | | (3/8) | (0/2) | (2/2) |
| anti-PD-1 Ab + | | 57% | 0 | 3 | 0 | 4 | 71% | 0% | 100% |
| Cabozantinib + | | | | | | | (5/7) | (0/4) | (4/4) |
| Celecoxib | | | | | | | | | |
| anti-PD-1 Ab + | | 100% | 0 | 0 | 1 | 6 | 100% | 0% | 100% |
| Cabozantinib + | | | | | | | (7/7) | (0/7) | (7/7) |
| Chidamide-k30 | | | | | | | | | |
| FIG. 9 | | | | | | | | | |
| Chidamide-k30 | 251 | 25% | 2 | 4 | 0 | 2 | 25% | 0% | |
| | | | | | | | (2/8) | (0/2) | |
| Lenvatinib | | 13% | 2 | 5 | 1 | 0 | 0% | 100% | |
| | | | | | | | (0/8) | (1/1) | |
| Lenvatinib + | | 38% | 2 | 3 | 0 | 3 | 38% | 0% | |
| Chidamide-k30 | | | | | | | (3/8) | (0/3) | |
| Axitinib | | 38% | 4 | 1 | 0 | 3 | 38% | 0% | |
| | | | | | | | (3/8) | (0/3) | |
| Axitinib + | | 50% | 1 | 3 | 0 | 4 | 50% | 0% | |
| Chidamide-k30 | | | | | | | (4/8) | (0/4) | |
| Regorafenib | | 25% | 2 | 4 | 0 | 2 | 13% | 50% | 50% |
| | | | | | | | (1/8) | (1/2) | (1/2) |
| Regorafenib + | | 88% | 0 | 1 | 0 | 7 | 88% | 0% | 71% |
| Chidamide-k30 | | | | | | | (7/8) | (0/7) | (5/7) |
| Cabozantinib | | 43% | 2 | 2 | 0 | 3 | 14% | 67% | 100% |
| | | | | | | | (1/7) | (2/3) | (3/3) |
| Cabozantinib + | | 75% | 0 | 2 | 1 | 5 | 75% | 17% | 100% |
| Chidamide-k30 | | | | | | | (6/8) | (1/6) | (6/6) |
| FIG. 10 | | | | | | | | | |
| anti-PD-1 Ab | 218 | 11% | 5 | 3 | 0 | 1 | 22% | 0% | 100% |
| | | | | | | | (2/9) | (0/1) | (1/1) |
| anti-PD-1 Ab + | | 44% | 2 | 3 | 1 | 3 | 33% | 50% | 67% |
| Cabozantinib | | | | | | | (3/9) | (2/4) | (2/3) |
| anti-PD-1 Ab + | | 0% | 4 | 5 | 0 | 0 | 0% | | |
| Regorafenib | | | | | | | (0/9) | | |
| anti-PD-1 Ab + | | 56% | 3 | 1 | 0 | 5 | 44% | 20% | 100% |
| Chidamide-k30 + | | | | | | | (4/9) | (1/5) | (5/5) |
| Celecoxib | | | | | | | | | |
| anti-PD-1 Ab + | | 56% | 0 | 4 | 0 | 5 | 56% | 0% | 100% |
| Cabozantinib + | | | | | | | (5/9) | (0/5) | (5/5) |
| Chidamide-k30 | | | | | | | | | |
| anti-PD-1 Ab + | | 33% | 0 | 6 | 0 | 3 | 78% | 0% | 100% |
| Regorafenib + | | | | | | | (7/9) | (0/3) | (3/3) |
| Chidamide-k30 | | | | | | | | | |

*The relapse/recurrence was defined as when having tumor growth at least 5 fold in mice with CR or PR response after first tumor assessment.
^{#}Mice resistant to CT26 re-challenge.
Response evaluation criteria: fold change of tumor size compared to baseline
PD: x □ 5;
SD: 1 □ x < 5;
PR: 0.5 □ x < 1;
CR: x < 0.5

TABLE 4
The anti-cancer activities of ICIs combined with TKIs plus HDAC inhibitors in CT26 tumor-bearing mice model.

| | | | | | | | | | | | | | | |
|---|---|---|---|---|---|---|---|---|---|---|---|---|---|---|
| | Initial | | | | | | | | | | | | | |
| | tumor | | | | | | | | | | | Survival | | |
| | volume | ORR | | | | | ORR | | | | | rate | Relapse* | Immunity^{#} |
| Regimens | (mm³) | (%) | PD | SD | PR | CR | (%)^{&} | PD^{&} | SD^{&} | PR^{&} | CR^{&} | (%) | (recurrence) | (rechallenge) |

FIG. 11A,
B, C, and D

| | | | | | | | | | | | | | | |
|---|---|---|---|---|---|---|---|---|---|---|---|---|---|---|
| anti-PD-1 | 243 | 0% | 6 | 4 | 0 | 0 | 0% | 8 | 2 | 0 | 0 | 0% | | |
| Ab | | | | | | | | | | | | (0/10) | | |
| anti-PD-1 | | 30% | 0 | 7 | 1 | 2 | 60% | 1 | 3 | 2 | 4 | 60% | 0% | 100% |
| Ab + | | | | | | | | | | | | (6/10) | (0/3) | (3/3) |
| Regorafenib + | | | | | | | | | | | | | | |
| Chidamide- | | | | | | | | | | | | | | |
| k30 | | | | | | | | | | | | | | |
| anti-PD-1 | | 40% | 4 | 2 | 1 | 3 | 40% | 4 | 2 | 0 | 4 | 40% | 0% | 100% |
| Ab + | | | | | | | | | | | | (4/10) | (0/4) | (4/4) |
| Regorafenib + | | | | | | | | | | | | | | |
| Vorinostat | | | | | | | | | | | | | | |
| anti-PD-1 | | 40% | 5 | 1 | 1 | 3 | 50% | 5 | 0 | 0 | 5 | 50% | 0% | 100% |
| Ab + | | | | | | | | | | | | (5/10) | (0/4) | (4/4) |
| Regorafenib + | | | | | | | | | | | | | | |
| Entinostat | | | | | | | | | | | | | | |
| Figure HE, | | | | | | | | | | | | | | |
| F, G, and H | | | | | | | | | | | | | | |
| anti-PD-1 | | 0% | 6 | 4 | 0 | 0 | 0% | 8 | 2 | 0 | 0 | 0% | | |
| Ab | | | | | | | | | | | | (0/10) | | |
| Cabozantinib + | | 60% | 1 | 3 | 0 | 6 | 60% | 3 | 1 | 0 | 6 | 60% | 0% | 100% |
| Chidamide- | | | | | | | | | | | | (6/10) | (0/6) | (6/6) |
| k30 | | | | | | | | | | | | | | |
| anti-PD-1 | | 30% | 0 | 7 | 1 | 2 | 60% | 1 | 3 | 2 | 4 | 60% | 0% | 100% |
| Ab + | | | | | | | | | | | | (6/10) | (0/3) | (3/3) |
| Regorafenib + | | | | | | | | | | | | | | |
| Chidamide- | | | | | | | | | | | | | | |
| k30 | | | | | | | | | | | | | | |
| anti-PD-L1 Ab+ | | 89% | 0 | 1 | 3 | 5 | 89% | 0 | 1 | 0 | 8 | 78% | 13% | 100% |
| Regorafenib + | | | | | | | | | | | | (7/9) | (1/8) | (8/8) |
| Chidamide- | | | | | | | | | | | | | | |
| k30 | | | | | | | | | | | | | | |
| anti-CTLA-4 | | 60% | 0 | 4 | 2 | 4 | 80% | 1 | 1 | 1 | 7 | 90% | 0% | 100% |
| Ab + | | | | | | | | | | | | (9/10) | (0/6) | (6/6) |
| Regorafenib + | | | | | | | | | | | | | | |
| Chidamide- | | | | | | | | | | | | | | |
| k30 | | | | | | | | | | | | | | |
| FIG. 11I, | | | | | | | | | | | | | | |
| J, K, and L | | | | | | | | | | | | | | |
| anti-PD-1 | | 0% | 6 | 4 | 0 | 0 | 0% | 8 | 2 | 0 | 0 | 0% | | |
| Ab | | | | | | | | | | | | (0/10) | | |
| anti-PD-1 | | 40% | 1 | 5 | 0 | 4 | 60% | 3 | 1 | 1 | 5 | 60% | 0% | 100% |
| Ab + | | | | | | | | | | | | (6/10) | (0/4) | (4/4) |
| Cabozantinib + | | | | | | | | | | | | | | |
| Chidamide- | | | | | | | | | | | | | | |
| k30 | | | | | | | | | | | | | | |
| anti-PD-1 | | 30% | 3 | 4 | 2 | 1 | 30% | 6 | 1 | 2 | 1 | 30% | 33% | 100% |
| Ab + | | | | | | | | | | | | (3/10) | (1/3) | (3/3) |
| Cabozantinib + | | | | | | | | | | | | | | |
| Vorinostat | | | | | | | | | | | | | | |
| anti-PD-1 | | 56% | 1 | 3 | 2 | 3 | 56% | 2 | 2 | 0 | 5 | 33% | 40% | 60% |
| Ab + | | | | | | | | | | | | (3/9) | (2/5) | (3/5) |
| Cabozantinib + | | | | | | | | | | | | | | |
| Entinostat | | | | | | | | | | | | | | |
| FIG. 11M, | | | | | | | | | | | | | | |
| N, O, and P | | | | | | | | | | | | | | |
| Anti-PD-1 | | 0% | 6 | 4 | 0 | 0 | 0% | 8 | 2 | 0 | 0 | 0% | | |
| Ab | | | | | | | | | | | | (0/10) | | |
| Cabozantinib + | | 60% | 1 | 3 | 0 | 6 | 60% | 3 | 1 | 0 | 6 | 60% | 0% | 100% |
| Chidamide- | | | | | | | | | | | | (6/10) | (0/6) | (6/6) |
| k30 | | | | | | | | | | | | | | |
| anti-PD-1 | | 40% | 1 | 5 | 0 | 4 | 60% | 3 | 1 | 1 | 5 | 60% | 0% | 100% |
| Ab + | | | | | | | | | | | | (6/10) | (0/4) | (4/4) |
| Cabozantinib + | | | | | | | | | | | | | | |
| Chidamide- | | | | | | | | | | | | | | |
| k30 | | | | | | | | | | | | | | |
| Anti-PD-L1 | | 60% | 0 | 4 | 3 | 3 | 60% | 2 | 2 | 1 | 5 | 60% | 17% | 100% |
| Ab + | | | | | | | | | | | | 6/10) | (1/6) | (6/6) |
| Cabozantinib + | | | | | | | | | | | | | | |
| Chidamide- | | | | | | | | | | | | | | |
| k30 | | | | | | | | | | | | | | |
| anti-CTLA-4 | | 90% | 0 | 1 | 1 | 8 | 90% | 0 | 1 | 0 | 9 | 90% | 0% | 100% |
| Ab + | | | | | | | | | | | | (9/10) | (0/9) | (9/9) |
| Cabozantinib + | | | | | | | | | | | | | | |
| Chidamide- | | | | | | | | | | | | | | |
| k30 | | | | | | | | | | | | | | |
| FIG. 11Q, | | | | | | | | | | | | | | |
| R, S, and T | | | | | | | | | | | | | | |
| anti-PD-1 | | 0% | 6 | 4 | 0 | 0 | 0% | 8 | 2 | 0 | 0 | 0% | | |
| Ab | | | | | | | | | | | | (0/10) | | |
| anti-PD-1 | | 30% | 0 | 7 | 1 | 2 | 60% | 1 | 3 | 2 | 4 | 60% | 0% | 100% |
| Ab + | | | | | | | | | | | | (6/10) | (0/3) | (3/3) |
| Regorafenib + | | | | | | | | | | | | | | |
| Chidamide- | | | | | | | | | | | | | | |
| k30 | | | | | | | | | | | | | | |
| anti-PD-1 | | 40% | 1 | 5 | 0 | 4 | 60% | 3 | 1 | 1 | 5 | 60% | 0% | 100% |
| Ab + | | | | | | | | | | | | (6/10) | (0/4) | (4/4) |
| Cabozantinib + | | | | | | | | | | | | | | |
| Chidamide- | | | | | | | | | | | | | | |
| k30 | | | | | | | | | | | | | | |
| anti-PD-1 | | 20% | 6 | 2 | 1 | 1 | 10% | 7 | 2 | 0 | 1 | 10% | 50% | 50% |
| Ab + RMC- | | | | | | | | | | | | (1/10) | (1/2) | (1/2) |
| 4550 + | | | | | | | | | | | | | | |
| Chidamide- | | | | | | | | | | | | | | |
| k30 | | | | | | | | | | | | | | |

*The relapse/recurrence was defined as when having tumor growth at least 5 fold in mice with CR or PR response after first tumor assessment.
^{&}the second tumor assessment 10 days after the last drug administration.
^{#}Mice resistant to CT26 re-challenge.
Response evaluation criteria: fold change of tumor size compared to baseline
PD: x □ 5;
SD: 1 □ x < 5;
PR: 0.5 □ x < 1;
CR: x < 0.5

### Example 7: The Comparison in Tumor Cell Population Between Anti-PD-1 Ab Plus Cabozantinib or Regorafenib Combination with or without Chidamide-k30 in CT26 Tumor-Bearing Mice

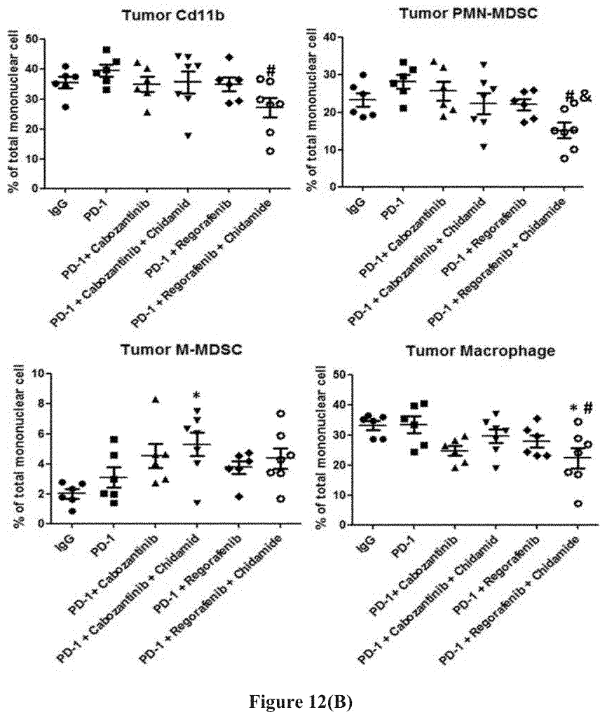

To determine whether treatment with combination of anti-PD-1 Ab combined with Cabozantinib/Regorafenib or triple combination of anti-PD-1 Ab combined with Cabozantinib/Regorafenib plus Chidamide-k30 affected myeloid-cell and T-cell population in tumors, tumor samples were isolated at day 9 after starting treatment, and immune cells were assessed by flow cytometry (FACS). As shown in FIG. 12, CD4⁺ T cells and Treg were significantly changed after anti-PD-1 Ab combined with Cabozantinib or Regorafenib with or without Chidamide-k30 treatment as shown in FIG. 12(A). The flow cytometry results suggested that anti-PD-1Ab combined with Cabozantinib or Regorafenib was powerful for the reduction of Treg cells which is conducive to activate immunity in tumors in response to tumor extension. In addition, Only PD-1 Ab+Regorafenib+Chidamide-k30 regimen resulted in significant increase in CD8⁺ T cell infiltration. About MDSC, it has been known that these myeloid-derived immature cells are often elevated in tumor-bearing hosts and have potent immunosuppressive activities. In comparison with other regimens, anti-PD-1 Ab combined with Regorafenib plus Chidamide-k30 treatment significantly decreased PMN-MDSC cells and tumor-associated macrophages (TAMs) in tumors as shown in FIG. 12(B), indicating that this triple combination was more likely the result of increasing tumor-infiltrating lymphocytes (TILs) by directing depletion of immune-suppressive cells PMN-MDSC cells and TAMs.

### Example 8: The Resistance to First Line Anti-PD-1 Ab Treatment was Overcome by Chidamide-k30 Combined with Cabozantinib/Regorafenib Plus Anti-CTLA-4 Ab Through Regulation of Gene Expression in the TME in CT-26 Tumor-Bearing Mice

As shown in FIG. 13, the gene expression regulated by treatment with different second line regimens to overcome the drug resistance to first line anti-PD-1 Ab treatment was analyzed. As shown in FIG. 13(A), the result demonstrated that the regimens of anti-CTLA-4 Ab combined with Chidamide-k30 plus Cabozantinib or Regorafenib were more powerful in inducing interferon gamma related gene expression than Chidamide-HCl salt combined with Celecoxib with or without anti-CTLA-4 Ab regimens and anti-CTLA-4 Ab alone. A similar result also demonstrated that anti-CTLA-4 Ab combined with Chidamide-k30 plus Cabozantinib or Regorafenib regimens significantly boosted the interferon-beta related gene expression in comparison with Chidamide-HCl salt combined with Celecoxib with or without anti-CTLA-4 Ab regimens and anti-CTLA-4 Ab alone as shown in FIG. 13(B). The gene expression related to T cell mediated cytotoxicity was analyzed as shown in FIG. 13(C). The regimens of anti-CTLA-4 Ab combined with Chidamide-k30 plus Cabozantinib or Regorafenib were more powerful in inducing the T cell mediated cytotoxicity related gene expression than Chidamide-HCl salt combined with Celecoxib or anti-CTLA-4 Ab alone. However, the angiogenesis activity related gene expression was significantly downregulated in anti-CTLA-4 Ab combined with Chidamide-k30 plus Regorafenib regimen as shown in FIG. 13(D). Taken together, all the regulation of gene expressions described above implied that for effectively overcoming the resistance caused by first-line anti-PD-1 Ab treatment the TME regulation involved the gene expression affected in the cells including immune cells in CT26 tumor.

### Example 9: Chidamide was a Key Component in the Regimens of Anti-PD-1 Ab Combined with Regorafenib/Cabozantinib Plus Chidamide-k30 for the Significant Regulation of Gene Expression in TME of CT26 Tumor-Bearing Mice

As shown in FIG. 14, gene expression analysis of CT26 tumors revealed the induction of a plethora of immune related pathways by Chidamide. As shown in FIG. 14(A), the comparison of anti-PD-1 Ab alone with anti-PD-1 Ab combined with Cabozantinib regimen showed that anti-PD-1 Ab combined with Cabozantinib regimen was more powerful in increasing the level of chemokine activity related gene expression. Furthermore, we unexpectedly found that anti-PD-1 Ab combined with Cabozantinib plus Chidamide-k30 regimen was more powerful in boosting chemokine activity related gene expression than anti-PD-1 Ab or anti-PD-1 Ab combined with Cabozantinib regimen. Similar results were also showed for anti-PD-1 Ab combined with Regorafenib plus Chidamide-k30 regimen. The immune response related gene expression was analyzed as shown in FIG. 14(B). Anti-PD-1 Ab combined with Cabozantinib plus Chidamide-k30 regimen was more powerful in boosting the immune response related gene expression than anti-PD-1 Ab alone or anti-PD-1 Ab combined with Cabozantinib regimen. Similar results were also shown for anti-PD-1 Ab combined with Regorafenib plus Chidamide-k30 regimen. Next, we analyzed the hallmark interferon gamma response related gene expression as shown in FIG. 14(C). Anti-PD-1 Ab combined with Cabozantinib plus Chidamide-k30 regimen was more powerful in increasing the hallmark interferon gamma response related gene expression than anti-PD-1 Ab alone or anti-PD-1 Ab combined with Cabozantinib regimen. Similar results were also shown to indicate that anti-PD-1 Ab combined with Regorafenib plus Chidamide-k30 regimen was more powerful in boosting the hallmark interferon gamma response related gene expression than anti-PD-1 Ab alone or anti-PD-1 Ab combined with Regorafenib regimen. The effect on downregulation of gene expression was analyzed. As shown in FIG. 14(D), the transmembrane receptor protein tyrosine kinase activity related gene expression was more significantly downregulated in anti-PD-1 Ab combined with Regorafenib plus Chidamide-k30 group in comparison with anti-PD-1 Ab alone or anti-PD-1 Ab combined with Regorafenib group. As shown in FIG. 14(E), the angiogenesis activity related gene expression was significantly downregulated in anti-PD-1 Ab combined with Regorafenib plus Chidamide-k30 group in comparison with anti-PD-1 Ab alone or anti-PD-1 Ab combined with Regorafenib group. The significant regulation of gene expression was observed at day 9 after starting treatment with combinations including Chidamide-k30, resulting in upregulation of chemokine activity, immune response, and hallmark interferon gamma related genes. These results demonstrated that anti-PD-1 Ab combined with Regorafenib/Cabozantinib plus Chidamide-k30 could complement and increase the efficacy of the immunotherapies in the CT26 tumor-bearing mice models. However, the downregulation of gene expression related to transmembrane receptor protein tyrosine kinase activity and angiogenesis activity was significant in anti-PD-1 Ab combined with Regorafenib plus Chidamide-k30 group when compared to anti-PD-1 Ab alone or anti-PD-1 Ab combined with Regorafenib group. Taken together, our data demonstrated and supported the rationale that ICIs combined with TKIs plus HDACis regimens possessed potent modulation activity in the TME to boost the immune response rate of CT26 tumor-bearing mice.

### Example 10: To Reconfirm the Anti-Cancer Activity of ICIs Combined with Tyrosine Kinase Inhibitors (TKIs) Plus Histone Deacetylase Inhibitors (HDACis) in CT26-Bearing Mice

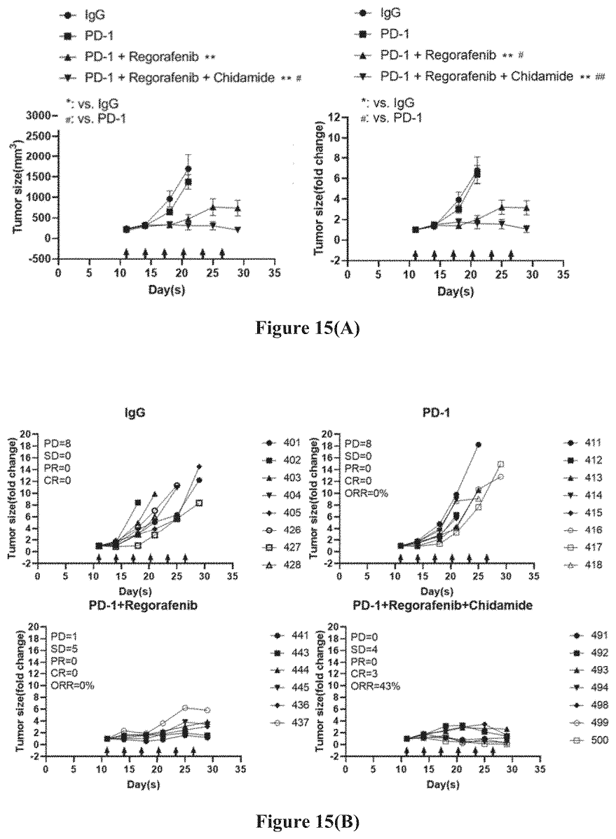

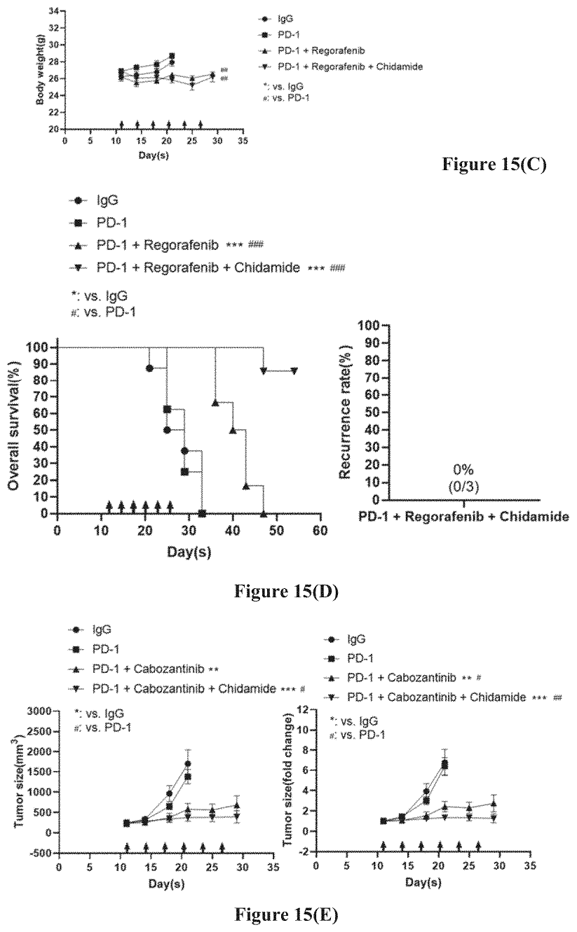

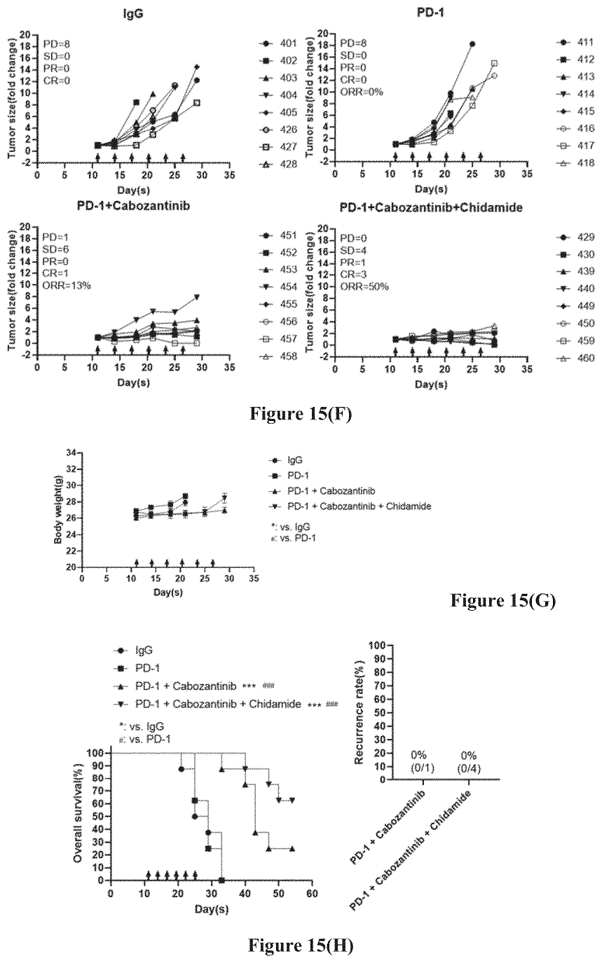

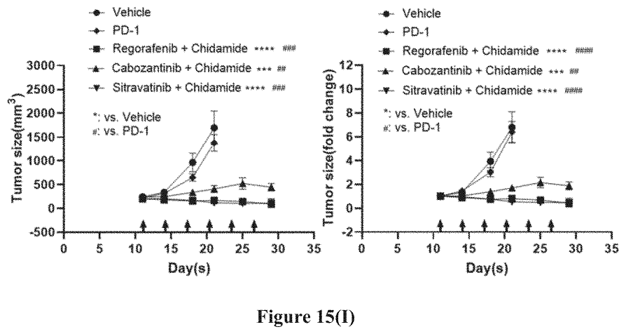

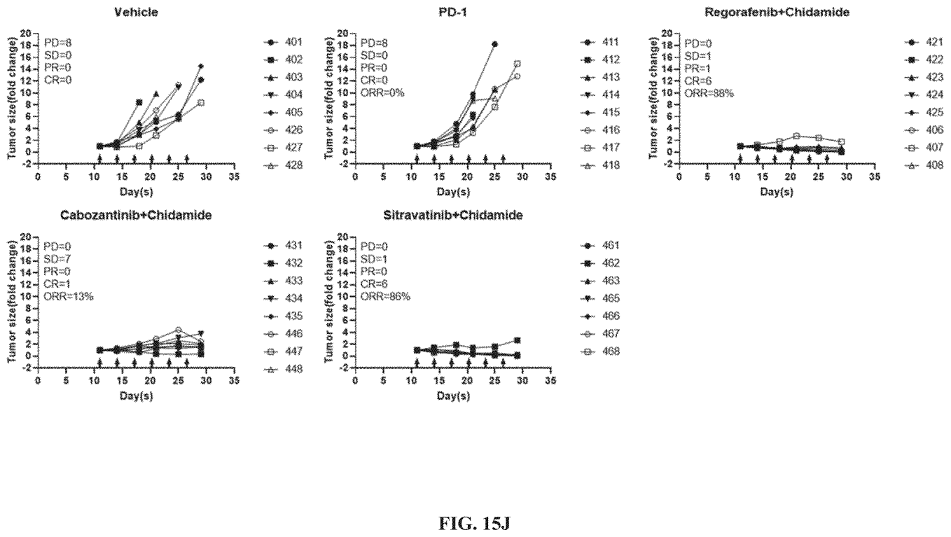

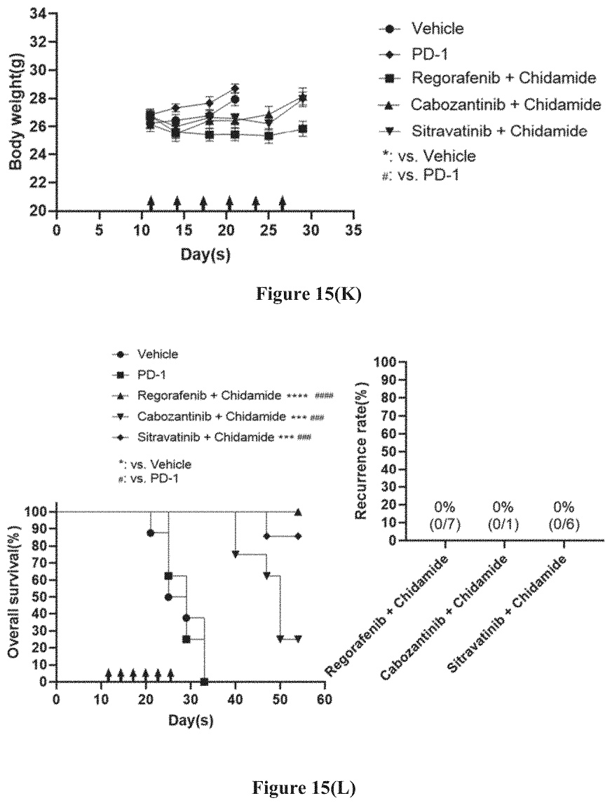

The anti-cancer activity of anti-PD-1 Ab combined with different TKIs plus Chidamide-k30 was further studied to reassure its potency in CT26 tumor-bearing mice. As shown from FIG. 15(A) to FIG. 15(D), the anti-PD-1 Ab combined with Regorafenib plus Chidamide-k30 regimen was evaluated. The result in FIG. 15(A) demonstrated that anti-PD-1 Ab (2.5 mg/kg) combined with Regorafenib (30 mg/kg) plus Chidamide-k30 (50 mg/kg) possessed more potent inhibition of tumor growth than anti-PD-1 Ab combined with Regorafenib. The individual tumor sizes (fold change) and ORR as shown in FIG. 15(B) indicated that anti-PD-1 Ab group achieved 8 PD, with the ORR (objective response rate) 0%; anti-PD-1 Ab combined with Regorafenib group achieved 5 SD and 1 PD, with the ORR 0%; anti-PD-1 Ab combined with Regorafenib plus Chidamide-k30 group achieved 3 CR and 4 SD, with the ORR 43%. The data show that anti-PD-1 Ab combined with Regorafenib plus Chidamide-k30 regimen has more potent antitumor activity than anti-PD-1 Ab combined with Regorafenib regimen. As shown in FIG. 15(C), anti-PD-1 Ab combined with Regorafenib and anti-PD-1 Ab combined with Regorafenib plus Chidamide-k30 groups initially dropped weight, but then eventually the mice body weight gradually recovered. The overall survival rate is shown in FIG. 15(D). Anti-PD-1 Ab combined with Regorafenib plus Chidamide-k30 group >anti-PD-1 Ab combined with Regorafenib group >anti-PD-1 Ab group. Not had recurrence occur in anti-PD-1 Ab combined with Regorafenib plus Chidamide-k30 group. The result also demonstrated that anti-PD-1 Ab combined with Regorafenib plus Chidamide-k30 was very potent to activate the immune system to avoid relapse. As shown from FIG. 15(E) to FIG. 15(H), the anti-PD-1 Ab combined with Cabozantinib plus Chidamide-k30 regimen was evaluated. The result in FIG. 15(E) demonstrated that anti-PD-1 Ab combined with Cabozantinib plus Chidamide-k30 possessed more potent inhibition of tumor growth than anti-PD-1 Ab combined with Cabozantinib. The individual tumor sizes (fold change) and ORR as shown in FIG. 15(F) indicated that anti-PD-1 Ab group achieved 8 PD, with the ORR (objective response rate) 0%; anti-PD-1 Ab combined with Cabozantinib group achieved 1 CR, 6 SD and 1 PD, with the ORR 13%; anti-PD-1 Ab combined with Cabozantinib plus Chidamide-k30 group achieved 3 CR, 1 PR and 4 SD, with the ORR 50%. The data showed that anti-PD-1 Ab combined with Cabozantinib plus Chidamide-k30 regimen has more potent antitumor activity than anti-PD-1 Ab combined with Cabozantinib regimen. As shown in FIG. 15(G), mice body weight did not significantly change. As shown in FIG. 15(H), in comparison with the other groups, anti-PD-1 Ab combined with Cabozantinib plus Chidamide-k30 regimen prolonged the survival, achieving 63% survival rate by day 54. Next, the recurrence rate in each group was evaluated. The result demonstrated that there was no relapse in anti-PD-1 Ab combined with Cabozantinib group and anti-PD-1 Ab combined with Cabozantinib plus Chidamide-k30 group. Next, we confirmed whether TKIs combination with HDACis possessed more potent activity of tumor growth inhibition than in the presence of ICIs. As shown from FIG. 15(I) to FIG. 15(L), the different TKIs combined with Chidamide-k30 regimens were evaluated. Both groups of Regorafenib combined with Chidamide-k30 and Sitravatinib combined with Chidamide-k30 show superior antitumor activity as shown in FIG. 15(I). The individual tumor sizes (fold change) and ORR as shown in FIG. 15(J) indicated that Regorafenib combined with Chidamide-k30 group achieved 6 CR, 1 PR and 1 SD with the ORR 88%; Cabozantinib combined with Chidamide-k30 group achieved 1 CR and 7 SD with the ORR 13%; Sitravatinib combined with Chidamide-k30 group achieved 6 CR and 1 SD with the ORR 86%. As shown in FIG. 15(K), mice body weight did not significantly change. As shown in FIG. 15(L), Regorafenib combined with Chidamide-k30 regimen significant prolonged the survival in comparison with the other groups, achieving 100% survival rate by day 54. This data suggested that Regorafenib combined with Chidamide-k30 possess potent immune regulation activity. The result of recurrence rate demonstrated that there was no relapse in different TKIs combined with Chidamide-k30 groups. The results of second tumor assessment and immunity/immune memory are shown in Table 5, which once confirmed the conclusion as stated in Examples 4 & 6 (FIG. 9 and Table 4). Treatment with regimen of Regorafenib combined with Chidamide-k30 in the absence of anti-PD-1 Ab showed incomplete or partial immunity in some mouse with complete tumor eradiation, and in the presence of anti-PD-1 showed a delayed development of immunity for the enhanced anti-cancer activity observed in second tumor assessment.

TABLE 5
The efficacy of HDAC inhibitors plus tyrosine kinase inhibitors with or without ICI in CT26 tumor-bearing mice model.

| | Initial | | | | | | | | | | | | | |
|---|---|---|---|---|---|---|---|---|---|---|---|---|---|---|
| | tumor | | | | | | | | | | | Survival | | |
| | volume | ORR | | | | | ORR | | | | | rate | Relapse* | Immunity^{#} |
| Regimens | (mm³) | (%) | PD | SD | PR | CR | (%)^{&} | PD^{&} | SD^{&} | PR^{&} | CR^{&} | (%) | (recurrence) | (rechallenge) |
| FIG. 15A, | | | | | | | | | | | | | | |
| B, C, and D | | | | | | | | | | | | | | |
| Anti-PD-1 | 227 | 0% | 8 | 0 | 0 | 0 | 0% | 8 | 0 | 0 | 0 | 0% | | |
| Ab | | | | | | | | | | | | | | |
| Anti-PD-1 | | 0% | 1 | 5 | 0 | 0 | 0% | 6 | 0 | 0 | 0 | 0% | | |
| Ab + | | | | | | | | | | | | | | |
| Regorafenib | | | | | | | | | | | | | | |
| Anti-PD-1 | | 43% | 0 | 4 | 0 | 3 | 86% | 1 | 0 | 1 | 5 | 86% | 0% | 100% |
| Ab + | | | | | | | | | | | | | (0/3) | (3/3) |
| Regorafenib + | | | | | | | | | | | | | | |
| Chidamide- | | | | | | | | | | | | | | |
| k30 | | | | | | | | | | | | | | |
| FIG. 15E, | | | | | | | | | | | | | | |
| F, G, and H | | | | | | | | | | | | | | |
| Anti-PD-1 | | 0% | 8 | 0 | 0 | 0 | 0% | 8 | 0 | 0 | 0 | 0% | | |
| Ab | | | | | | | | | | | | | | |
| Anti-PD-1 | | 13% | 1 | 6 | 0 | 1 | 13% | 5 | 2 | 0 | 1 | 25% | 0% | 100% |
| Ab + | | | | | | | | | | | | | (0/1) | (1/1) |
| Cabozantinib | | | | | | | | | | | | | | |
| Anti-PD-1 | | 50% | 0 | 4 | 1 | 3 | 50% | 1 | 3 | 0 | 4 | 63% | 0% | 100% |
| Ab + | | | | | | | | | | | | | (0/4) | (4/4) |
| Cabozantinib + | | | | | | | | | | | | | | |
| Chidamide- | | | | | | | | | | | | | | |
| k30 | | | | | | | | | | | | | | |
| FIG. 11I, | | | | | | | | | | | | | | |
| J, K, and L | | | | | | | | | | | | | | |
| Anti-PD-1 | | 0% | 8 | 0 | 0 | 0 | 0% | 8 | 0 | 0 | 0 | 0% | | |
| Ab | | | | | | | | | | | | | | |
| Regorafenib + | | 88% | 0 | 1 | 1 | 6 | 88% | 0 | 1 | 0 | 7 | 100% | 0% | 86% |
| Chidamide- | | | | | | | | | | | | | (0/7) | (6/7) |
| k30 | | | | | | | | | | | | | | |
| Cabozantinib + | | 13% | 0 | 7 | 0 | 1 | 25% | 3 | 3 | 1 | 1 | 25% | 0% | 100% |
| Chidamide- | | | | | | | | | | | | | (0/1) | (1/1) |
| k30 | | | | | | | | | | | | | | |
| Sitravatinib + | | 86% | 0 | 1 | 0 | 6 | 86% | 1 | 0 | 0 | 6 | 86% | 0% | 100% |
| Chidamide- | | | | | | | | | | | | | (0/6) | (6/6) |
| k30 | | | | | | | | | | | | | | |

*The relapse/recurrence was defined as when having tumor growth at least 5 fold in mice with CR or PR response after first tumor assessment.
^{&}The second tumor assessment 10 days after the last administration of second line treatment.
^{#}Mice resistant to CT26 re-challenge.
Response evaluation criteria: fold change of tumor size compared to baseline
PD: x □ 5;
SD: 1 □ x < 5;
PR: 0.5 □ x < 1;
CR: x < 0.5

While the present disclosure has been described in conjunction with the specific embodiments set forth above, many alternatives thereto and modifications and variations thereof will be apparent to those of ordinary skill in the art. All such alternatives, modifications and variations are regarded as falling within the scope of the present disclosure.

What is claimed is:

1. A method of inhibiting or treating a cancer in a subject in need thereof, wherein the method comprises administering to the subject a pharmaceutical combination comprising a histone deacetylase (HDAC) inhibitor or a pharmaceutically acceptable salt thereof and a tyrosine kinase inhibitor (TKI) or a pharmaceutically acceptable salt thereof, wherein the HDAC inhibitor or the pharmaceutically acceptable salt thereof and the tyrosine kinase inhibitor or the pharmaceutically acceptable salt thereof are co-administered simultaneously, separately or sequentially or co-administered in combination as a coformulation; optionally the pharmaceutical combination comprises an immune checkpoint inhibitor (ICI), the histone deacetylase (HDAC) inhibitor or the pharmaceutically acceptable salt thereof, and the tyrosine kinase inhibitor (TKI) or the pharmaceutically acceptable salt thereof; wherein the histone deacetylase (HDAC) inhibitor or the pharmaceutically acceptable salt thereof, the tyrosine kinase inhibitor (TKI) or the pharmaceutically acceptable salt thereof and the immune checkpoint inhibitor are co-administered simultaneously, separately or sequentially or co-administered in combination as a coformulation.

2. (canceled)

3. The method of claim 1, wherein the cancer is melanoma, head and neck cancer, merkel cell carcinoma, hepatocellular carcinoma, renal cell carcinoma, colorectal cancer, endometrial carcinoma, cervical cancer, esophageal squamous cell carcinoma, small cell lung cancer, non-small cell lung cancer, breast cancer, gastric carcinoma, esophagogastric junction carcinoma, classical Hodgkin lymphoma, Non-Hodgkin lymphoma, urothelial carcinoma, primary mediastinal large B-cell lymphoma, glioblastoma, pancreatic cancer, benign prostate hyperplasia, prostate cancer, ovarian cancer, chronic lymphocytic leukemia, Merkel cell carcinoma, acute myeloid leukemia, gallbladder cancer, cholangiocarcinoma, urinary bladder cancer, or uterine cancer.

4. The method of claim 1, wherein the cancer is an immune checkpoint inhibitor-resistant cancer or a cancer failure to respond to a cancer immunotherapy.

5. The method of claim 1, wherein the subject has not received a cancer therapy.

6. The method of claim 1, wherein the subject has received a cancer therapy but failed to the therapy.

7. The method of claim 1, wherein the HDAC inhibitor or the pharmaceutically acceptable salt thereof is a class I-selective HDAC inhibitor or pan-HDAC inhibitor which must inhibit class I HDAC.

8. The method of claim 1, wherein the HDAC inhibitor or the pharmaceutically acceptable salt thereof is a benzamide class of HDAC inhibitor.

9. The method of claim 1, wherein the HDAC inhibitor or the pharmaceutically acceptable salt thereof is Chidamide, Entinostat, Vorinostat, Romidepsin, Panobinostat, Belinostat, Valproic acid, Mocetinostat, Abexinostat, Pracinostat, Resminostat, Givinostat Quisinostat, Domatinostat, Quisnostat, CUDC-101, CUDC-907, Pracinostat, Citarinostat, Droxinostat, Abexinostat, Ricolinostat, Tacedinaline, Fimepinostat, Tubacin, Resminostat, ACY-738, Tinostamustine, Tubastatin A, Givinostat or Dacinostat, or a pharmaceutically acceptable salt thereof.

10. The method of claim 1, wherein the TKI or the pharmaceutically acceptable salt thereof is an inhibitor of receptor tyrosine kinase.

11. The method of claim 1, wherein the TKI or the pharmaceutically acceptable salt thereof is an inhibitor of vascular endothelial growth factor receptor (VEGFR).

12. The method of claim 1, wherein the TKI or the pharmaceutically acceptable salt thereof is Cabozantinib, Regorafenib, Axitinib, Afatinib, Nintedanib, Crizotinib, Alectinib, Trametinib, Dabrafenib, Sunitinib, Ruxolitinib, Vemurafenib, Sorafenib, Ponatinib, Encorafenib, Brigatinib, Pazopanib, Dasatinib, Imatinib, Lenvatinib, Vandetanib, surufatinib or Sitravatinib, or a pharmaceutically acceptable salt thereof.

13. The method of claim 1, wherein the immune checkpoint inhibitor is an anti-cytotoxic T-lymphocyte antigen-4 (CTLA-4) antibody, anti-programmed cell death protein 1 (PD-1) antibody, an anti-programmed death-ligand 1 (PD-L1) antibody, an anti-T-cell immunoglobulin and mucin domain-3 (TIM-3) antibody, anti-B- and T-lymphocyte attenuator (BTLA) antibody, anti-V-domain Ig containing suppressor of T-cell activation (VISTA) antibody, an anti-lymphocyte activation gene-3 (LAG-3) antibody, A2AR (adenosine A2A receptor inhibitor, CD276 (B7 Homolog 3) inhibitor or antibody, VCTN1 inhibitor or antibody, KIR (killer-cell immunoglobulin-like receptor) inhibitor or antibody.

14. The method of claim 1, wherein the immune checkpoint inhibitor is pembrolizumab, lambrolizumab, pidilizumab, nivolumab, durvalumab, avelumab, or atezolizumab.

15. The method of claim 1, wherein the amounts of the HDAC inhibitor and the TKI in the composition range from about 10% (w/w) to about 70% (w/w) and about 10% (w/w) to about 70% (w/w), respectively.

16. The method of claim 1, wherein the amount of immune checkpoint inhibitor in the composition ranges from about 0.5% (w/w) to about 20% (w/w).

17. The method of claim 1, wherein the pharmaceutical composition further comprises one or more additional anti-cancer agents.

18. The method of claim 1, whereby the administering or co-administering of said pharmaceutical composition of co-formulation results in overcoming immune suppression in tumor microenvironment or stimulating immune response.

19. A pharmaceutical combination comprising a histone deacetylase (HDAC) inhibitor or a pharmaceutically acceptable salt thereof, a tyrosine kinase inhibitor (TKI) or a pharmaceutically acceptable salt thereof, and at least one of an immune checkpoint inhibitor and additional anti-cancer agents.

20. The pharmaceutical combination of claim 19, wherein the amounts of the HDAC inhibitor and the TKI in the pharmaceutical combination range from about 10% (w/w) to about 70% (w/w) and about 10% (w/w) to about 70% (w/w), respectively.

21. The pharmaceutical combination of claim 19, wherein the immune checkpoint inhibitor in the pharmaceutical composition ranges from about 0.5% (w/w) to about 20% (w/w).
